SEQUENCE LISTING (16 sequences)

SEQ ID NO: 1 - DNA, 18 bp, synthetic construct
  source 1..18: mol_type = other DNA
ggtgttgcaaagacctag

SEQ ID NO: 2 - DNA, 21 bp, synthetic construct
  source 1..21: mol_type = other DNA
gcagtccttttaattaccctg

SEQ ID NO: 3 - RNA, 20 nt, synthetic construct
  source 1..20: mol_type = other RNA
aatactgaatgaccgccatc

SEQ ID NO: 4 - RNA, 20 nt, synthetic construct
  source 1..20: mol_type = other RNA
aggcttccgcaacttgcatg

SEQ ID NO: 5 - RNA, 20 nt, synthetic construct
  source 1..20: mol_type = other RNA
attgcccatcttgtcgtctc

SEQ ID NO: 6 - RNA, 20 nt, synthetic construct
  source 1..20: mol_type = other RNA
gggtggaaagtaatagtcga

SEQ ID NO: 7 - RNA, 20 nt, synthetic construct
  source 1..20: mol_type = other RNA
gcactaccagagctaactca

SEQ ID NO: 8 - DNA, 24 bp, synthetic construct
  source 1..24: mol_type = other DNA
cagcagaaatgattgcactattga

SEQ ID NO: 9 - DNA, 22 bp, synthetic construct
  source 1..22: mol_type = other DNA
agagtcatccctgcttcataac

SEQ ID NO: 10 - DNA, 20 bp, synthetic construct
  source 1..20: mol_type = other DNA
cagcacagggaggaatcaat

SEQ ID NO: 11 - DNA, 20 bp, synthetic construct
  source 1..20: mol_type = other DNA
gtgtcgtagaaatgccacct

SEQ ID NO: 12 - DNA, 22 bp, synthetic construct
  source 1..22: mol_type = other DNA
ggagaactactccggaccttat

SEQ ID NO: 13 - DNA, 23 bp, synthetic construct
  source 1..23: mol_type = other DNA
gggtggaaagtaatagtcgatgg

SEQ ID NO: 14 - DNA, 18 bp, synthetic construct
  source 1..18: mol_type = other DNA
ctggccaaggtcatccat

SEQ ID NO: 15 - DNA, 18 bp, synthetic construct
  source 1..18: mol_type = other DNA
ttctgggtggcagtgatg

SEQ ID NO: 16 - protein, 8 aa, Gallus gallus
SIINFEKL

(19) United States
(12) Patent Application Publication
(10) Pub. No.: US 2024/0400991 A1
(43) Pub. Date: Dec. 5, 2024

(54) IMMUNE CELLS WITH REDUCED ANDROGEN RECEPTOR (AR) LEVEL, AND METHODS OF THEIR USE TO ENHANCE ANTI-CANCER THERAPY

(71) Applicant: Oregon Health & Science University, Portland, OR (US)

(72) Inventors: Amy Elizabeth Moran, Portland, OR (US); Fanny Catherine Polesso, Portland, OR (US)

(73) Assignee: Oregon Health & Science University, Portland, OR (US)

(21) Appl. No.: 18/694,839

(22) PCT Filed: Sep. 23, 2022
(86) PCT No.: PCT/US2022/076964

§ 371 Date: Mar. 22, 2024

Related U.S. Application Data
(60) Provisional application No. 63/319,712, filed on Mar. 14, 2022.
(60) Provisional application No. 63/275,882, filed on Nov. 4, 2021.
(60) Provisional application No. 63/248,408, filed on Sep. 24, 2021.

(51) Int. Cl. C12N 5/0783; A61K 35/17; C12N 5/0781; C12N 5/0784; C12N 5/0786; C12N 9/22

(52) U.S. Cl. CPC C12N 5/0636; A61K 35/17; C12N 5/0635; C12N 5/0639; C12N 5/0645; C12N 5/0646; C12N 9/22; C12N 2510/00

(57) ABSTRACT

The present disclosure relates generally to dis-arming immune cell product sensitivity to androgens. Provided herein are methods, systems, and compositions for disarming androgen receptor (AR) activity in immune cells, for instance in the context of CAR-T cell therapy, allogenic T cell therapy, DC vaccines, macrophage therapy, myeloid/macrophage antigen receptor therapy, NK cell therapy, and so forth. Different methods for reducing AR function are described.

### CROSS-REFERENCE TO RELATED APPLICATIONS

This application is the 371 National Phase of International Application No. PCT/US22/76964, filed on Sep. 23, 2022, which claims priority to and the benefit of the earlier filing of U.S. Provisional Application No. 63/248,408, filed on Sep. 24, 2021; U.S. Provisional Application No. 63/275,882, filed on Nov. 4, 2021; and U.S. Provisional Application No. 63/319,712, filed on Mar. 14, 2022. Each of these earlier-filed applications is incorporated by reference herein in its entirety.

### STATEMENT REGARDING FEDERALLY SPONSORED RESEARCH OR DEVELOPMENT

This invention was made with government support under CA097186 awarded by the National Institutes of Health. The government has certain rights in the invention.

### INCORPORATION BY REFERENCE OF SEQUENCE LISTING

A computer readable text file, entitled “0046-0059US_SeqList.xml” created on or about Mar. 22, 2024, with a file size of 15,142 bytes, contains the sequence listing for this application and is hereby incorporated by reference in its entirety.

### FIELD OF THE DISCLOSURE

The present disclosure relates generally to immunotherapy treatments, for instance improved immunotherapy treatments that involve cells with reduced androgen receptor and/or function.

### BACKGROUND OF THE DISCLOSURE

Cell therapies for the treatment of cancer have revolutionized cancer care and the clinical management of many cancer subtypes. Importantly, these therapies are often agnostic to the sex of the patient or the levels of circulating androgens. In general, androgens suppress the immune system.

In advanced prostate cancer patients, immunotherapy treatments (such as checkpoint blockade) have largely failed. Androgens have pleotropic functions in prostate cancer; they are potent drivers of cancer cell growth and can suppress T cell function and IFNg. In metastatic prostate cancer, intratumor androgen levels can remain high. Androgen deprivation therapy, the backbone of clinical care in prostate cancer, is classically administered to inhibit tumor cell growth.

Prostate cancer (PCa) is the second leading cause of cancer-related deaths in men in the United States. PCa is initially managed with surgery, radiation, androgen antagonists (e.g., bicalutamide), and surgical or chemical castration. However, the relapsed or metastatic disease post-castration (castration-resistant prostate cancer or CRPC) has a poor prognosis with most patients dying within two years (Karantanos et al., European Urology 67:470-479, 2015). Innovative treatment approaches are urgently needed to treat CRPC patients.

### SUMMARY OF THE DISCLOSURE

Described herein is the discovery that androgen receptor (AR) activity restrains the function of immune cells with anti-tumor potential. By reducing androgen receptor signaling, for instance by genetic repression/deletion (for instance, using CRISPR/Cas9 or a similar editing system) or pharmacologic inhibition, the effectiveness of cell therapies for the treatment of cancers can be improved. This approach is amenable to enhancing cell therapy products independent of secondary immune-modulating therapy (i.e., checkpoint inhibitors/engages) and/or with immunotherapy.

By limiting androgen receptor activity, multiple cell products that have enhanced anti-cancer function (and proliferation) can be generated for cancer immunotherapy. Some of the individual populations include:
Reduction in T cell intrinsic androgen receptor level and/or function to improve T cell function: allogenic T-cell therapy, adoptive cell therapy, autologous T cell therapy, CAR-T;
Reduction in T cell intrinsic androgen receptor level and/or function to reduce regulatory T cell suppression;
Reduction in myeloid and/or macrophage intrinsic androgen receptor levels to improve engineered macrophage therapy;
Reduction in dendritic cell intrinsic androgen receptor level and/or function to improve antigen presentation;
Reduction in natural killer cell intrinsic androgen receptor level and/or function to improve cell killing; and
Reduction in B cell intrinsic androgen receptor and/or function improves antibody maturation.

In general, this disclosure provides methods and systems whereby leukocyte intrinsic androgen receptor (AR) expression is genetically or pharmacologically reduced, thereby enhancing the function of the cell therapy. Examples include: Chimeric antigen receptor T cells would be targeted to reduce AR expression and would have enhanced anti-tumor function.

Dendritic cell therapy treated with a drug that down regulates AR will demonstrate increased antigen presentation and enhanced anti-tumor immunity. Because AR is known to stabilize regulatory T cells, targeted reduction of AR in regulatory T cells will destabilize their suppressive function and enable (more) effective immunotherapy. In representative embodiments, immune cell products are modified prior to infusion and/or cell therapy is co-treated with AR inhibitors to enhance cell function.

All leukocytes express AR and therefore, cell product targeted with treatment to reduce AR expression are identifiable by quantitative evaluation of AR expression. This is routinely performed by assays to detect gene and protein expression. Baseline AR expression prior to cell product modification (either genetically or pharmacologically) can be established followed by AR expression measurements after modification.

Embodiments are provided in which expression of AR is reduced or “knocked out” genetically, by modification of sequence encoding AR or by genetic manipulation that otherwise down regulates or degrades mRNA expressed from the AR gene. An exemplary description of using CRISPR/Cas9 to knock out AR in immune cells is described in Example 1 and the accompanying figures.

An embodiment is a method of improving function of an immune cell or population of immune cells, including reducing intrinsic androgen receptor (AR) expression or activity level.

Also described is use of an immune cell engineered to have reduced androgen receptor (AR) expression.

Another embodiment is a method, including: dual inhibition of AR and at least one immune checkpoint target, such as dual inhibition of AR and PD-1/PD-L1 checkpoint, to reduce tumor growth and improve survival; treatment with androgen deprivation therapy (ADT), AR inhibition (e.g., with enzalutamide, bicalutamide, flutamide, abiraterone, or the like) or AR degradation (e.g., with AR-PROTACs), and an immune checkpoint inhibitor (such as an agent that provides PD-1 blockade) in a subject that has become, for instance, refractory to PD-1/checkpoint therapy; or increasing the effectiveness of PD-1 blockade, by reducing AR activity in CD8 T cells in patients refractory to PD-1/checkpoint therapy.

Yet another embodiment is a method to increase production of effector cytokines (such as granzyme B, IFNγ and/or TNFα) in a subject, including treating the subject with: an androgen reduction therapy (such as ADT, an AR inhibitor, an AR degrader, or a combination thereof); and an anti-PD-L1 agent or other agent that provides checkpoint blockade.

Also described are methods to increase effectiveness of immune therapies and immune-mediated tumor including treating a subject with a combination of AR inhibitor with an epigenetic modifier (such as an EZH2 inhibitor or LSD1 inhibitor), alone or with immunotherapy.

Another embodiment is a method, including treating a subject with an AR inhibitor, an epigenetic modifier, and immune cell therapy (such as autologous immune cell transfer).

Also described is a biomarker allowing identification of patients who could achieve a clinical benefit from checkpoint blockade, or to distinguish responsive from non-responsive patients, essentially as described herein.

Also provided is use of the biomarkers described herein to develop or modify a treatment regimen for a subject.

Another embodiment is a method of improving immune cell-mediated therapeutic treatment by reducing expression and/or function of androgen receptor, essentially as described herein.

### BRIEF DESCRIPTION OF DRAWINGS

Some of the drawings submitted herein or in the accompanying document(s) may be better understood in color. Applicant consider the color versions of the drawings as part of the original submission and reserve the right to present color images of the drawings in later proceedings.

FIGS. 1A-1D. The immune landscape of tumors from patients with mCRPC prior to checkpoint therapy. (FIG. 1A) Schematic of mCRPC patients' clinical treatment and biopsy timing prior to checkpoint therapy with waterfall plot of PSA response after checkpoint blockade of patients on which scRNAseq was performed. (FIG. 1B) UMAP (uniform manifold approximation and projection) plot of all cells that passed quality control. Cells are colored based on cell types. (FIG. 1C) UMAP plot of all T/NK lymphocytes collected in this study with cells colored by cell types. (FIG. 1D) Heatmap showing scaled expression of top 20 cell markers ranked by fold change in each cluster in FIG. 1C. A list of representative genes is shown. R, responder; NR, non-responder.

FIGS. 2A-2C. Clinical trial schema of patients enrolled and details on biopsy location and genomics. (FIG. 2A) Clinical trial study scheme. (FIG. 2B) Per-patient tumor mutations are shown in a table with each row representing an individual participant on study, and each column representing the unique participant identifier (StudyID), the participant's response to study treatment (Outcome), the site of biopsied tissue specimen analyzed by whole exome sequencing (Biopsy Site), the relative (%) change in PSA with treatment (PSA change), the number of somatic variants detected in that tumor specimen (Somatic_variant_count), and the coverage-adjusted tumor mutational burden defined as the Somatic_variant_count/#Mbp genome covered by 6 reads (Coverage_adj_mtl_burden). (FIG. 2C) Comparison of the somatic variant counts (left) or coverage-adjusted tumor mutational burdens (right) for study responders (R) versus non-responders (NR); NS represents no significant difference detected; mean values are depicted as bold horizontal lines.

FIGS. 3A-3H. CD8 T cell subset associated with response to checkpoint therapy in mCRPC patients. (FIG. 3A) Representative flow cytogram for sorting tumor-associated leukocytes prior to scRNAseq. (FIG. 3B) UMAP of merged cells from all patients scRNAseq libraries. (FIG. 3C) Stack bar graph showing the % of cells per sample for immune cell clusters across each patient biopsy. Percentage was calculated out of all immune cells without accounting for tumor cells. Two-tailed Student's t-test. (FIG. 3D, 3E) Boxplots comparing the % of cells per sample for immune cell clusters between responders and non-responders. Percentage was calculated out of all immune cells without accounting for tumor cells. Two-tailed Student's t-test. (FIG. 3F) Heatmap showing the expression of CTLA4, HAVCR2, PDCD1, TIGIT, CD274, LAG3, ICOS, BTLA in various T cell clusters. (FIG. 3G) Pathways enriched in dysfunctional CD8 T cells. (FIG. 3H) Percentage of cells co-expressing a combination of PDCD1, LAG3, HAVCR2, CTLA4, TNFRSF4, and TIGIT in dysfunctional CD8 T cells.

FIGS. 4A-4J. CD8 T cell signature associated with response implicates a functional role for AR. (FIG. 4A) UMAP plot showing the two distinct CD8 T cells states identified using unsupervised k-means clustering. (FIG. 4B) Heatmap displaying the scaled expression of top 20 most discriminative genes ranked by fold change between CD8_k1 and CD8_k2 cells. (FIG. 4C) UMAP plot showing CD8 T cells colored by response and non-response patient groups. (FIG. 4D) Heatmap showing the scaled expression of top 20 most differentially expressed genes ranked by fold change between CD8+ T cells from responders and non-responders. Two-tailed Student's t-test. (FIG. 4E) Master Regulator analysis was used to identify transcription factors whose activity is different between CD8 T cells from responders vs. those from non-responders. The top 20 transcription factors predicted to be most activated (red) or deactivated (blue) in CD8 T cells from responders vs. those from non-responders are shown. The targets of each transcription factor are shown as tick marks with red vertical lines representing positive targets and blue vertical lines negative targets of a given transcription factor. Each row also illustrates the P value and inferred differential activity (Act) for each transcription factor. (FIG. 4F) Pathway analysis illustrating the molecular signatures distinguishing CD8 T cells from responders and non-responders; these are predicted pathways and their directionality (up/red or down/blue) based on differentially expressed genes (DEGs) from FIGS. 4B/4D. (FIG. 4G, 4H) Correlation analysis between CD8_k1.vs.k2 signature score (FIG. 4G) or CD8 R.vs.NR signature score (FIG. 4H) with AR activity in bulk RNA sequencing data derived from individual patients on the trial; DEGs from FIGS. 4B/4D were used to create a ‘signature’ termed CD8 k1 or CD8 R herein, and then a correlation was draw n between ‘if yes’ for the signature what is the ‘score’. Each dot=1 patient. Red color represents responders and blue color non-responders. Two-tailed Pearson correlation. (FIG. 4I) Immunohistochemical (IHC) staining of AR in treatment nave prostate cancer patients (clockwise from top left: Gleason Grade 4+3, 3+4, 3+3, 3+3). Arrows indicate positive expression within immune cells. (FIG. 4J) Kinetics of AR expression in purified human CD8 T cells after TCR stimulation. k1, k-means=1; k2, k-means=2; R, responder; NR, non-responder.

FIGS. 5A-5D. Expression of various marker genes associated with CD8 T cytotoxicity and exhaustion. (FIG. 5A) Venn diagram and contingency table showing the significant overlap between CD8_R and CD8_k1 (Top, P<0.0001) and between CD8_NR and CD8_k2 (Bottom, P<0.0001). All cells represented all the cells that passed quality control in this study, as shown in FIGS. 1A and 1B. (FIG. 5B) Percentage of CD8_k1 or CD8_k2 clusters per sample in responders and non-responders. Two-tailed Fisher's exact test. (FIG. 5C, 5D) Violin plot comparing the gene expression in CD8_k1 and CD8_k2 (FIG. 5B), and CD8_R and CD8_NR (FIG. 5C). R, responder; NR, non-responder.

FIGS. 6A-6C. CD4_k1 is not associated with response. (FIG. 6A) UMAP plot showing the two distinct CD4 T cells states identified using k-means clustering. (FIG. 6B) UMAP plot showing CD4 T cells colored by response and non-response patient groups. (FIG. 6C) Percentage of CD4_k1 or CD4_k2 clusters per sample in responders and non-responders. Two-tailed Student's t-test. R, responder; NR, non-responder.

FIGS. 7A-7F. Survival data following orthotopic PPSM implantation and enzalutamide+anti-PD-L1 treatment. (FIG. 7A) AR expression by qPCR in mouse CD8 T cells, as compared with PPSM and 688 m AR positive and negative control cell lines, respectively. (FIG. 7B) Summary table of the experiments described in FIG. 8A. (FIG. 7C) 12-14 wk old male mice were castrated and PPSM tumor cells were injected orthotopically in the anterior lobe of the prostate. One week later, animals were treated with enzalutamide or enzalutamide+α-PDL1 (5 animals per group). 4 weeks post tumor inoculation, tumors were harvested and measured. (FIGS. 7D-7E) PPSM tumor bearing animals were treated along the same timeline as FIG. 8A but in the absence of ADT. Average tumor growth (FIG. 7D) and Survival curves (FIG. 7E) of tumor bearing animals treated with combination therapy in the presence or absence of ADT (8 animals per group). (FIG. 7F) Survival curves of PPSM tumor bearing animals orchiectomized or not at day 7 (5 animals per group).

FIGS. 8A-8N. Dual inhibition of AR and PD-1/PD-L1 improves T cell function and overall survival in tumor models. (FIG. 8A) Experimental design for tumor survival experiments. (FIG. 8B, 8C) Average tumor growth (FIG. 8B) and survival curves (FIG. 8C) of PPSM tumor bearing animals treated with different treatment combination. data combined 7 independent experiments, 6 to 8 animals per group. (FIG. 8D, 8E) Average tumor growth (FIG. 8D) and survival curves (FIG. 8E) of PPSM tumor bearing animals treated with combination therapy and α-CD8 depleting antibody (8 animals per group). (FIG. 8F) Survival data of MCA-205 tumor bearing animals treated as in FIG. 8D with the different treatment combinations (8 animals per group). (FIGS. 8G-8L) PPSM tumor bearing animals were treated as in FIG. 8A, tumors were harvested the day after the 3^{rd} α-PD-L1 treatment and CD8 tumor infiltrating cells were analyzed by flow cytometry. (FIG. 8G) Representative cytograms showing CD44 and Granzyme B expression in CD8 T cells in the tumor, and (FIG. 8H) summarized percent Granzyme B+CD8 T cells in the tumor. (FIG. 8I) Representative cytograms showing IFNγ and TNFα expression in CD8 T cells in the tumor. Digested tumors were stimulated for 5 hours with PMA-lonomycin, and ICCS was performed. Summarized percent (FIG. 8J) IFNγ, (FIG. 8K) TNFα (FIG. 8L) and IFNγ+TNFα+ expressing TIL CD8 in the different treatment groups. (FIGS. 8G-8L) Data representative of 3 independent experiments with 3 to 6 animals per group. (FIG. 8M) Experimental design for differential gene expression queried with nCounter mouse immunology panel using nCounter SPRINT profiler. (FIG. 8N) GSEA plot illustrating the association of AR-inhibited genes in mouse TIL with CD8_R patient single cells (nominal P=0.045). GSEA, Gene set enrichment analysis; TIL, tumor infiltrated lymphocytes; R, responder; NR, non-responder. Unpaired student t-test *P<0.05, **P<0.01, ***P<0.001, ****P<0.0001.

FIGS. 9A-9O. Phenotyping data of tumor infiltrating CD8 T cells from orthotopic PPSM tumors, degarelix treated, and enzalutamide+anti-PD-L1 only treated. In FIGS. 9A-9D, PPSM tumor bearing animals were treated as in FIG. 8A. CD8 T cell number (FIG. 9A), Ki67 expression (FIG. 9B), PD-1 MFI (FIG. 9C) and CD44 MFI (FIG. 9D) in CD8 T cells in the tumor one day after the 3^{rd} treatment with α-PD-L1. In FIGS. 9E-9G, PPSM tumor cells were surgically injected orthotopically in the prostate, and orchiectomy was performed. One week later, animals were treated with enzalutamide only or enzalutamide+α-PD-L1 (5 animals per group). 4 weeks post tumor inoculation, tumors were harvested and processed for flow cytometry. Graphs show percent IFNγ+(FIG. 9E), TNFα+(FIG. 9F) and IFNγ+TNFα+double producing (FIG. 9G) CD8 T cells in the tumor. In FIGS. 9H-9L, PPSM tumor bearing animals were treated with degarelix (2 doses, d7 and d21 post tumor inoculation), enzalutamide (started at d7) and α-PD-L1 (3 doses, d7, 10, 13). Tumors were harvested on day 14 and processed for flow cytometry. Graphs show percent Ki67+(FIG. 9H), IFNγ+(FIG. 9I), TNFα+(FIG. 9J), IFNγ+ TNFα+(FIG. 9K) and Granzyme B+(FIG. 9L) CD8 T cells in the tumor. In FIGS. 9M-9O, PPSM tumor bearing animals were treated with the same timeline as in FIG. 8A, but with enzalutamide+α-PD-L1 or ADT+α-PD-L1. Tumors were harvested the day after the 3^{rd} dose of α-PD-L1 and processed for flow cytometry. (FIG. 9M) Percent Granzyme B+CD8 T cells in the tumor. (FIG. 9N) Representative flow cytogram showing IFNγ and TNFα expression in CD8 T cells in the tumor, and (FIG. 9O) Summarized percent IFNγ+TNFα+CD8 T cells in the tumor. Unpaired student t-test *P<0.05, **P<0.01, ***P<0.001.

FIGS. 10A-10C. Enzalutamide treatment leads to increased cytokine production in tumor specific T cells. (FIG. 10A) Experimental design. Male or female Ripm-OVA animals were implanted with MCA-OVA tumors. Male animals were treated with degarelix at time of tumor inoculation. At d7 animals were adoptively transferred with OT1; Thy1.1 CD8 T cells, and half of the animals were started on enzalutamide treatment. 12 days post adoptive transfer, tumors were harvested, and TIL were stimulated with SIINFEKL peptide (SEQ ID NO: 16) and ICCS was performed. (FIG. 10B, 10C) Representative flow cytograms showing CD44 and IFNγ expression in OTI T cells in the tumor, and summarized % IFNγ+ and PD-1 MFI in OTI in the tumor in males (FIG. 10B) and females (FIG. 10C). Unpaired student t-test *p<0.05.

FIGS. 11A-11L. Suppressing androgen receptor function in T cells promotes IFNG activity. (FIG. 11A) The open chromatin regions (OCRs) containing predicted androgen receptor elements (AREs) in Ifng and Gzmbloci are shown. (FIGS. 11B, 11C) AR ChIP-qPCR data shows specific binding of AR to the predicted AREs Ifng and Gzmb genes in activated mouse T cells (FIG. 11B) and the effect of enzalutamide on AR binding to these AREs (FIG. 11C). (FIG. 11D, 11E) GSEA plot illustrating the significant enrichment of IFNG response in Ar-KO T cells versus control. NES, normalized enrichment score (FIG. 11D) and (FIG. 11E) box plot showing the enrichment scores of the human CD8_R.vs.NR signature in the mouse CD8 T cells of Ar-KO versus control. The statistical p-value was determined by the two-tailed Student's t-test. (FIGS. 11F-111) Schematic of LCMV experiment (FIG. 11F), percent IFNγ+P14 (FIG. 11G), total numbers of P14 (FIG. 11H) and PD-1 MFI in P14 (FIG. 11I) in the LN at day 32 post infection. (FIGS. 11J-11L) Correlation analysis between CD8 R.vs.NR signature score and AR activity or IFNG pathway activity (left and middle columns) and between AR activity and IFNG pathway activity (right column) in (FIG. 11J) a larger mCRPC patient cohort (n=99) and (k-l) two metastatic melanoma cohorts (FIG. 11K) n=42 and (FIG. 11L) n=27 (Quigley et al., Cell 174:758-769. e759, 2018). Two-tailed Pearson correlation. Tex, exhausted CD8 T cell; TIL, tumor infiltrated lymphocytes; OCR: open chromatin region; Unpaired student t-test *p<0.05, **p<0.01.

FIGS. 12A-12F. T cell deletion of AR. (FIG. 12A) Experimental design of the generation of AR-KO CD8 T cells in vitro using CRISPR/Cas9. Purified CD8 T cells were electroporated with Cas9/gRNA complex (NT or AR gRNA) and put in culture in vitro for 3 days in plates coated with anti-CD3 and anti-CD28. 3 days later, stimulated cells were harvested, and RNA was extracted or cells were restimulated in vitro for 5 hours with PMA/lonomycin, followed by ICCS. (FIG. 12B) AR mRNA levels by qPCR in CD8 T cells electroporated with non-targeting (NT) or AR gRNA/Cas9 after 3 days of in vitro stimulation. (FIG. 12C) Representative flow cytograms of IFNγ and TNFα expression after restimulation with PMA/lonomycin. (FIG. 12D) Experimental design for the generation of AR-KO P14 CD8 T cells and LCMV infection model. (FIG. 12E) AR mRNA levels in purified P14 at day 7 post adoptive transfer. (FIG. 12F) PD1 MFI and percent IFNγ+ in P14 in the blood at day 7 post adoptive transfer.

FIG. 13 illustrates one embodiment of the disclosure, in which AR expression of cell therapy products is modified prior to infusion. Data described herein also demonstrate that pharmacologic inhibition of AR enhances the cell product. These approaches are amendable to leukocytes independent of differentiation state. Though illustrated here with a genetic knockout (using CRISPR/Cas9), additional embodiments employ pharmacological intervention to directly or indirectly reduce the function of AR in target cells.

FIGS. 14A-14D. Immunohistochemical staining of Androgen Receptor (AR) in lymphocytes within the prostate cancer microenvironment. Each panel represents a different prostate cancer: (FIG. 14A) Gleason Grade: 4+3; (FIG. 14B) Gleason Grade: 3+4; (FIG. 14C) Gleason Grade: 3+3; (FIG. 14D) Gleason Grade: 3+3. The cancer cells have distinct glandular appearance. In contrast, the lymphocytes are dark blue. AR expression (brown) is seen in the nuclei of cancer cells and lymphocytes. Solid arrows indicate positive expression of AR within the lymphocytes; Dashed arrows indicate positive expression of AR within prostate cancer cells.

FIGS. 15A-15C. Immunohistochemical staining of Androgen Receptor (AR) in lymphocytes. (FIG. 15A) Lymphoid follicle in tonsil, (FIG. 15B) lymphoid aggregate in colon, and (FIG. 15C) lymphoid infiltrate in benign prostate. The lymphoid cells are small, dark blue cells with minimal cytoplasm. AR expression (brown) is seen in lymphocytes, and is indicated by the solid arrows (in FIG. 15C, the dashed arrows indicate positive expression of AR within benign prostate cells).

FIGS. 16A-16C. Graphs illustrating AR activity compared to a CD8 R (responder) signature (FIG. 16A); INFG pathway activity by CD8 R signature (FIG. 16B); and IFNG activity inversely correlated with AR expression in CAR-T infusion product (FIG. 16C).

Strong and statistically significant correlations were found between AR activity and the CD8 R versus NR signature score; IFNG pathway score and CD8 R versus NR signature score; and between IFNG pathway score and AR activity.

FIGS. 17A-17C. Graphs illustrating sex segregated cell (F=female; M=male); AR activity compared to a CD8 R (responder) signature (FIG. 17A); IFNG pathway activity by CD8 R signature (FIG. 17B); and IFNG activity inversely correlated with AR expression in CAR-T infusion product (FIG. 17C).

### REFERENCE TO SEQUENCE LISTING

The nucleic acid and/or amino acid sequences described herein are shown using standard letter abbreviations, as defined in 37 C.F.R. § 1.822. Only one strand of each nucleic acid sequence is shown, but the complementary strand is understood as included in embodiments where it would be appropriate. A computer readable text file, entitled “0046-0059US_SeqList.xml” created on or about Mar. 22, 2024, with a file size of 15,142 bytes, contains the sequence listing for this application and is hereby incorporated by reference in its entirety.

SEQ ID NOs: 1 and 2 are forward and reverse
primers used to amplify OCRs in Ifng using qPCR:
GGTGTTGCAAAGACCTAG (SEQ ID NO: 1),
GCAGTCCTTTTAATTACCCTG (SEQ ID NO: 2).
SEQ ID NOs: 3-6 are sgRNA targeting the murine
AR gene: sgRNA 1:
AATACTGAATGACCGCCATC (SEQ ID NO: 3);
sgRNA 2:
AGGCTTCCGCAACTTGCATG (SEQ ID NO: 4);
sgRNA 3:
ATTGCCCATCTTGTCGTCTC (SEQ ID NO: 5);
sgRNA 4:
GGGTGGAAAGTAATAGTCGA (SEQ ID NO: 6).
SEQ ID NO: 7 is a mouse genome nontargeting
Ctrl sgRNA:
5′-GCACUACCAGAGCUAACUCA-3′.
SEQ ID NOs: 8 and 9 are forward and reverse
qPCR primer sequences for human AR:
5′CAGCAGAAATGATTGCACTATTGA3′ (SEQ ID NO: 8);
5′AGAGTCATCCCTGCTTCATAAC3′ (SEQ ID NO: 9).
SEQ ID NOs: 10 and 11 are forward and reverse
qPCR primer sequences human SDHA:
5′CAGCACAGGGAGGAATCAAT3′ (SEQ ID NO: 10);
5′GTGTCGTAGAAATGCCACCT3′ (SEQ ID NO: 11).
SEQ ID NOs: 12 and 13 are forward and reverse
mouse qPCR primer sequences for Ar.
5′GGAGAACTACTCCGGACCTTAT3′ (SEQ ID NO: 12);
5′GGGTGGAAAGTAATAGTCGATGG3′ (SEQ ID NO: 13).
SEQ ID NOs: 14 and 15 are forward and reverse
mouse qPCR primer sequences for
Gapdh: 5′CTGGCCAAGGTCATCCAT3′ (SEQ ID NO: 14);
5′TTCTGGGTGGCAGTGATG3′ (SEQ ID NO: 15).

SEQ ID NO: 16 is the amino acid sequence of the ovalbumin peptide SIINFEKL, a signal peptide presented by the MHC class I H-2 kb allele for stimulation of T cells.

### DETAILED DESCRIPTION

Provided herein are methods of improving function of an immune cell or population of immune cells, including: reducing intrinsic androgen receptor (AR) expression or activity level. By way of example of such method of embodiments (or any other method embodiment), the immune cell or a cell within the population of immune cells can be: a T-cell (including a CAR-T cell), a myeloid cell, a macrophage, a dendritic cell, a natural killer cell, or an B-cell.

In example method embodiments the cell is a T cell, and reducing the intrinsic AR level improves T cell function in allogenic T cell therapy, adoptive cell therapy, CAR-T therapy, or autologous T cell therapy. In example method embodiments the cell is a T cell, and reducing the intrinsic AR level reduces regulatory T cell suppression. In example method embodiments the cell is a myeloid cell or a macrophage, and reducing the intrinsic AR level improves engineered macrophage therapy. In example method embodiments the cell is a dendritic cell, and reducing the intrinsic AR level improves antigen presentation. In example method embodiments the cell is a natural killer cell, and reducing the intrinsic AR level improves cell killing. In example method embodiments the cell is a B cell, and reducing the intrinsic AR level improves antibody maturation.

Also described are immune therapeutic cells genetically modified to reduce expression of intrinsic androgen receptor (AR). By way of example, in examples of such embodiments the cell is a T-cell, a myeloid cell, a macrophage, a dendritic cell, a natural killer cell, or an B-cell. Another embodiment is a cell formulation including an immune therapeutic cell genetically modified to reduce expression of intrinsic AR, and a pharmaceutically acceptable carrier.

A further embodiment is a method of genetically-modifying an immune cell to provide an improved immune therapy product, the method including inhibiting expression and/or activity of androgen receptor (AR) in the immune cell.

A further embodiment is a method of overcoming inhibition of an immunotherapy response, including reducing expression and/or activity of androgen receptor (AR) in a cell used in the immunotherapy.

A further embodiment is a method of improving the therapeutic effectiveness of an immunotherapy, including reducing expression and/or activity of androgen receptor (AR) in a cell used in the immunotherapy.

A further embodiment is a method of improving efficacy of immune checkpoint blockade therapy, including conducting the immune checkpoint blockade therapy (1) with a genetically-modifying cell in which the genetic modification results in reduction in androgen receptor (AR) expression and/or activity, or (2) in the presence of an AR antagonist. For instance, in examples the immune checkpoint blockade therapy includes cancer treatment, such as treatment of prostate cancer.

Another embodiment is use of an immune cell engineered to have reduced androgen receptor (AR) expression and/or activity to treat cancer, such as prostate cancer.

By way of example in any of the provided method or use embodiments, reducing expression and/or activity of androgen receptor (AR) in the cell includes one or more of: modification of a genomic sequence encoding AR, such as through targeted gene editing/disruption; post-transcriptional inhibition of AR expression, such as through induction of RNAi; and/or pharmacological inhibition of AR activity or function. For instance, in some cases the targeted gene editing includes Clustered Regularly Interspaced Short Palindromic Repeats/CRISPR-associated (CRISPR/Cas) nuclease editing of a genomic sequence encoding AR, zinc finger nuclease (ZFN) editing of a genomic sequence encoding AR, or transcription activator like effector nuclease (TALEN) editing of a genomic sequence encoding AR.

Another embodiment is a biomarker allowing identification of patients who could achieve a clinical benefit from immune checkpoint blockade (such as PD-1 blockade, TIGIT blockade, LAG3 blockade, or TIM3 blockade), or to distinguish responsive from non-responsive patients, essentially as described herein. Examples of this biomarker embodiment include: one or more of T cell intrinsic AR activity, or CD8_R signature, or IFNG pathway activity. Specific examples include for instance at least one gene that is described herein as up-regulated in AR KO T cells (e.g., one or more of H2-Eb1, H2-Aa, 1113, Igfbp4, Rsad2, Rpl28-ps1, Gm1821, Tmem106a, Rpl36a-ps2, Ceacam-ps1, Gpr15, Trp53cor1, Rnaset2a, Gm10288, AL607105.1, and/or Emp1) and/or at least one gene that is described herein as down-regulated in AR KO T cells (e.g., one or more of Traj24, Tm4sf19, Snord32a, AC159308.5, Gm15564, Gm36931, Gm45599, Gm6939, Gm26642, Arg1, Cxcl3, Gm590, Sema6b, 58304111N06Rik, Gm37768, Trajl1, Gm38319, AC133083.2, Traj22, Gm45803, Gm17606, Klhl35, Gm26835, AC132474.1, Ankrd55, Ccl22, Gm37105, and/or |110). Also described are uses of a provided biomarker, for instance to develop or modify a treatment regimen for a subject.

Yet another embodiment is a method of improving immune-cell mediated therapeutic treatment by reducing expression and/or function of androgen receptor, essentially as described herein.

Additional embodiments are described herein.

### Select Terms

Androgen Receptor: The androgen receptor (AR) is a type of nuclear receptor that is a member of the steroid hormone receptor family of molecules. The androgen receptor is activated by binding of either of the androgenic hormones, testosterone or dihydrotestosterone. The androgen receptor mediates the physiologic effects of androgens by binding to DNA sequences that influence transcription or androgen-responsive genes.

Intrinsic Androgen Receptor (e.g., intrinsic androgen receptor expression or activity level): The term “intrinsic” means occurring as a natural part of something or belonging to the essential nature of something. “Intrinsic androgen receptor expression” or “intrinsic androgen receptor activity level” refers to the baseline or native expression or activity level of the androgen receptor, for instance in a selected standard such as a individual with a healthy level of androgen receptor expression/activity, or an average amount/value determined by analyzing a population of individuals believed to have a non-disease state androgen receptor activity or expression level, etc. In embodiments, the intrinsic level refers to the level (of AR expression and/or activity) in the absence of a therapeutic intervention intended to decrease or increase that level.

Biomarker: A molecule that is associated either quantitatively or qualitatively with a biological activity or function (e.g., impaired or unimpaired or operable T-cell immune effector function). Examples of biomarkers include polynucleotides, such as a gene product, RNA or RNA fragment, polynucleotide copy number alterations (e.g., DNA copy numbers); proteins, polypeptides, and fragments of a polypeptide or protein; carbohydrates, and/or glycolipid-based molecular markers; polynucleotide or polypeptide modifications (e.g., posttranslational modifications, phosphorylation, DNA methylation, acetylation, and other chromatin modifications, glycosylation, etc.). In certain embodiments, a “biomarker” means a molecule/compound that is differentially present (i.e., increased or decreased) in a sample as measured/compared against the same marker in another sample or suitable control/reference. In other embodiments, a biomarker can be differentially present in a sample as measured/compared against the other markers in same or another sample or suitable control/reference. In further embodiments, one or more biomarkers can be differentially present in a sample as measured/compared against other markers in the same or another sample or suitable control/reference and against the same markers in another sample or suitable control/reference. In yet another embodiment, a biomarker can be differentially present in a sample from a subject or a group of subjects having a first phenotype (e.g., having a disease or condition) as compared to a sample from a subject or group of subjects having a second phenotype (e.g., not having the disease or condition or having a less severe version of the disease or condition).

Cancer: A disease or condition in which abnormal cells divide without normal regulation or control, and are able to invade other tissues. Cancer cells spread to other body parts through the blood and lymphatic systems. Cancer is a term for many diseases. There are more than 100 different types of cancer in humans. Most cancers are named after the organ in which they originate. For instance, a cancer that begins in the colon can be termed a colon cancer. However, the characteristics of a cancer, especially with regard to the sensitivity of the cancer to therapeutic compounds, are not limited to the organ in which the cancer originates. A cancer cell is any cell derived from any cancer, whether in vitro or in vivo. A malignant tumor is characterized by abnormal or uncontrolled cell growth. Other features often associated with cancer include metastasis, interference with the normal functioning of neighboring cells, release of cytokines or other secretory products at abnormal levels and suppression or aggravation of inflammatory or immunological response, invasion of surrounding or distant tissues or organs, such as lymph nodes, etc.

Chimeric antigen receptor: As used herein, “Chimeric antigen receptor” or “CAR” refers to an engineered protein that includes (i) an extracellular domain that includes a moiety that binds a target antigen; (ii) a transmembrane domain; and (iii) an intracellular signaling domain that sends activating signals when the CAR is stimulated by binding of the extracellular binding moiety with a target antigen.

A T cell that has been genetically engineered to express a chimeric antigen receptor may be referred to as a CAR T cell. Thus, for example, when certain CARs are expressed by a T cell, binding of the CAR extracellular binding moiety with a target antigen can activate the T cell. CARs are also known as chimeric T cell receptors or chimeric immunoreceptors.

Disrupt: With respect to a gene, this term refers to preventing the formation of a functional gene product. A gene product is functional only if it fulfills its normal (wild-type) functions. Disruption of the gene prevents expression of a functional factor encoded by the gene and includes an insertion, deletion, or substitution of one or more bases in a sequence encoded by the gene and/or a promoter and/or an operator that is necessary for expression of the gene in the animal. The disrupted gene may be disrupted by, e.g., removal of at least a portion of the gene from a genome of the animal, alteration of the gene to prevent expression of a functional factor encoded by the gene, an interfering RNA, or expression of a dominant negative factor by an exogenous gene. Materials and methods of genetically modifying hematopoietic stem/progenitor cells are detailed in U.S. Pat. No. 8,518,701; U.S. 2010/0251395; and U.S. 2012/0222143.

Endogenous: Characteristic of a substance, such as a molecule, cell, tissue, or organ (e.g., a hematopoietic stem cell or a cell of hematopoietic lineage, such as a megakaryocyte, thrombocyte, platelet, erythrocyte, mast cell, myeloblast, basophil, neutrophil, eosinophil, microglial cell, granulocyte, monocyte, osteoclast, antigen-presenting cell, macrophage, dendritic cell, natural killer cell, T-lymphocyte, or B-lymphocyte) that is found naturally in a particular organism, such as a human patient.

Engineered (cell): As used herein, the term “engineered” refers to the aspect of having been manipulated by the hand of man. For example, a polynucleotide is considered to be “engineered” when two or more sequences, that are not linked together in that order in nature, are manipulated by the hand of man to be directly linked to one another in the engineered polynucleotide. Those of skill in the art will appreciate that an “engineered” nucleic acid or amino acid sequence can be a recombinant nucleic acid or amino acid sequence, and can be referred to as “genetically engineered.” In some embodiments, an engineered polynucleotide includes a coding sequence and/or a regulatory sequence that is found in nature operably linked with a first sequence but is not found in nature operably linked with a second sequence, which is, in the engineered polynucleotide, operably linked in with the second sequence by the hand of man. In some embodiments, a cell or organism is considered to be “engineered” or “genetically engineered” if it has been manipulated so that its genetic information is altered (e.g., new genetic material not previously present has been introduced, for example by transformation, mating, somatic hybridization, transfection, transduction, or other mechanism, or previously present genetic material is altered or removed, for example by substitution, deletion, or mating). As is common practice and is understood by those of skill in the art, progeny or copies, perfect or imperfect, of an engineered polynucleotide or cell are typically still referred to as “engineered” even though the direct manipulation was of a prior entity.

Exogenous: Characteristic of a substance, such as a molecule, cell, tissue, or organ (e.g., a hematopoietic stem cell or a cell of hematopoietic lineage, such as a megakaryocyte, thrombocyte, platelet, erythrocyte, mast cell, myeloblast, basophil, neutrophil, eosinophil, microglial cell, granulocyte, monocyte, osteoclast, antigen-presenting cell, macrophage, dendritic cell, natural killer cell, T-lymphocyte, or B-lymphocyte) that is not found naturally in a particular organism, such as a human patient. Exogenous substances include those that are provided from an external source to an organism or to cultured matter extracted therefrom.

Expression: As used herein, “expression” of a polynucleotide (for example, a gene or a transgene) refers to the process by which the coded information of a transcriptional unit (including, e.g., gDNA or cDNA) is converted into an operational, non-operational, or structural part of a cell, often including the synthesis of a protein. Gene expression can be influenced by external signals; for example, exposure of a cell, tissue, or organism to an agent that increases or decreases gene expression. Expression of a gene can also be regulated anywhere in the pathway from DNA to RNA to protein. Regulation of gene expression occurs, for example, through controls acting on transcription, translation, RNA transport and processing, degradation of intermediary molecules such as mRNA, or through activation, inactivation, compartmentalization, or degradation of specific protein molecules after they have been made, or by combinations thereof. Gene expression can be measured at the RNA level or the protein level by any method known in the art, including, without limitation, northern blot, RT-PCR, western blot, or in vitro, in situ, or in vivo protein activity assay(s).

Expression or activity level: As used herein, the phrase “expression or activity level” refers to a quantitative or qualitative measure of a species. The species can be a gene, expression product, protein, chemical, or cell. Expression or activity level can calculated as a ratio of a species of interest to a reference level.

Gene Deletion/Gene Knockout: A “gene deletion” or “gene knockout” refers to rendering a specific gene or family of genes inoperable or inactive, and can be carried out by a number of different genetic techniques. In particular embodiments, a gene deletion reduces or eliminates expression of a polypeptide encoded by the target gene(s). In particular embodiments, the expression of the gene(s) is substantially reduced or eliminated. Substantially reduced means that the expression of the gene(s) is reduced by at least 80%, at least 90%, at least 95%, or at least 98% when compared to an endogenous level of expression of the gene. Expression of gene(s) can be determined by a suitable technique (e.g., by measuring transcript or expressed protein levels).

Hematopoietic progenitor cells: A category of cells that includes pluripotent cells capable of differentiating into several cell types of the hematopoietic system, including, without limitation, granulocytes, monocytes, erythrocytes, megakaryocytes, B-cells and T-cells, among others. Hematopoietic progenitor cells are committed to the hematopoietic cell lineage and generally do not self-renew. Hematopoietic progenitor cells can be identified, for example, by expression patterns of cell surface antigens, and include cells having the following immunophenotype: Lin− KLS+ Flk2− CD34+. Hematopoietic progenitor cells include short-term hematopoietic stem cells, multi-potent progenitor cells, common myeloid progenitor cells, granulocyte-monocyte progenitor cells, and megakaryocyte-erythrocyte progenitor cells. The presence of hematopoietic progenitor cells can be determined functionally, for example, by detecting colony-forming unit cells, e.g., in complete methylcellulose assays, or phenotypically through the detection of cell surface markers using flow cytometry and cell sorting assays described herein and known in the art.

Hematopoietic stem cells (HSCs): A category of immature blood cells having the capacity to self-renew and to differentiate into mature blood cells containing diverse lineages including but not limited to granulocytes (e.g., promyelocytes, neutrophils, eosinophils, basophils), erythrocytes (e.g., reticulocytes, erythrocytes), thrombocytes (e.g., megakaryoblasts, platelet producing megakaryocytes, platelets), monocytes (e.g., monocytes, macrophages), dendritic cells, microglia, osteoclasts, and lymphocytes (e.g., NK cells, B-cells and T-cells). Such cells may include CD34+ cells. CD34+ cells are immature cells that express the CD34 cell surface marker. In humans, CD34+ cells are believed to include a subpopulation of cells with the stem cell properties defined above, whereas in mice, HSCs are CD34−. In addition, HSCs also refer to long term repopulating HSCs (LT-HSC) and short term repopulating HSCs (ST-HSC). LT-HSCs and ST-HSCs are differentiated, based on functional potential and on cell surface marker expression. For example, human HSCs are CD34+, CD38−, CD45RA−, CD90+, CD49F+, and lin− (negative for mature lineage markers including CD2, CD3, CD4, CD7, CD8, CD10, CD11B, CD19, CD20, CD56, CD235A). In mice, bone marrow LT-HSCs are CD34−, SCA-1+, C-kit+, CD135−, Slamfl/CD150+, CD48−, and lin− (negative for mature lineage markers including Ter119, CD11 b, Gr1, CD3, CD4, CD8, B220, IL7ra), whereas ST-HSCs are CD34+, SCA-1+, C-kit+, CD135−, Slamfl/CD150+, and lin− (negative for mature lineage markers including Ter119, CD11b, Gr1, CD3, CD4, CD8, B220, IL7ra). In addition, ST-HSCs are less quiescent and more proliferative than LT-HSCs under homeostatic conditions. However, LT-HSC have greater self-renewal potential (i.e., they survive throughout adulthood, and can be serially transplanted through successive recipients), whereas ST-HSCs have limited self-renewal (i.e., they survive for only a limited period of time, and do not possess serial transplantation potential). Any of these HSCs can be used in the methods described herein. ST-HSCs are particularly useful because they are highly proliferative and thus, can more quickly give rise to differentiated progeny.

Hematopoietic stem cell functional potential: The functional properties of hematopoietic stem cells which include 1) multi-potency (which refers to the ability to differentiate into multiple different blood lineages including, but not limited to, granulocytes (e.g., promyelocytes, neutrophils, eosinophils, basophils), erythrocytes (e.g., reticulocytes, erythrocytes), thrombocytes (e.g., megakaryoblasts, platelet producing megakaryocytes, platelets), monocytes (e.g., monocytes, macrophages), dendritic cells, microglia, osteoclasts, and lymphocytes (e.g., NK cells, B-cells and T-cells), 2) self-renewal (which refers to the ability of hematopoietic stem cells to give rise to daughter cells that have equivalent potential as the mother cell, and further that this ability can repeatedly occur throughout the lifetime of an individual without exhaustion), and 3) the ability of hematopoietic stem cells or progeny thereof to be reintroduced into a transplant recipient whereupon they home to the hematopoietic stem cell niche and re-establish productive and sustained hematopoiesis.

Heterologous: The term “heterologous,” as applied to polynucleotides and/or polyribonucleotides herein, means of different origin. For example, if a host cell is transformed with a polynucleotide that does not occur in the untransformed host cell in nature, then that polynucleotide is heterologous (and exogenous) to the host cell. Furthermore, different elements (e.g., promoters, enhancers, coding sequences, and terminators) of an expression cassette may be heterologous to one another and/or to the transformed host. Heterologous polynucleotides herein also specifically include a polynucleotide that is identical in sequence to a polynucleotides already present in a host cell, but that is linked to a different regulatory sequence and/or are present at a different copy number in the host cell.

Human antibody: An antibody in which substantially every part of the protein (for example, all CDRs, framework regions, CL, CH domains (e.g., CH1, CH2, CH3), hinge, and VL and VH domains) is substantially non-immunogenic in humans, with only minor sequence changes or variations. A human antibody can be produced in a human cell (for example, by recombinant expression) or by a non-human animal or a prokaryotic or eukaryotic cell that is capable of expressing functionally rearranged human immunoglobulin (such as heavy chain and/or light chain) genes. When a human antibody is a single chain antibody, it can include a linker peptide that is not found in native human antibodies. For example, an Fv can contain a linker peptide, such as two to about eight glycine or other amino acid residues, which connects the variable region of the heavy chain and the variable region of the light chain. Such linker peptides are considered to be of human origin. Human antibodies can be made by a variety of methods known in the art including phage display methods using antibody libraries derived from human immunoglobulin sequences. Human antibodies can also be produced using transgenic mice that are incapable of expressing functional endogenous immunoglobulins, but which can express human immunoglobulin genes (see, for example, PCT Publication Nos. WO 1998/24893; WO 1992/01047; WO 1996/34096; WO 1996/33735; U.S. Pat. Nos. 5,413,923; 5,625,126; 5,633,425; 5,569,825; 5,661,016; 5,545,806; 5,814,318; 5,885,793; 5,916,771; and 5,939,598).

Humanized antibody: A non-human antibody that contains minimal sequences derived from non-human immunoglobulin. In general, a humanized antibody contains substantially all of at least one, and typically two, variable domains, in which all or substantially all of the CDR regions correspond to those of a non-human immunoglobulin. All or substantially all of the FW regions may also be those of a human immunoglobulin sequence. The humanized antibody can also contain at least a portion of an immunoglobulin constant region (Fc), typically that of a human immunoglobulin consensus sequence. Methods of antibody humanization are known in the art and have been described, for example, in Riechmann et al. (Nature 332:323-327, 1988); U.S. Pat. Nos. 5,530,101; 5,585,089; 5,693,761; 5,693,762; and 6,180,370.

Immune cells: The phrase immune cell encompasses any and all cells that are part of an immune response, including both myeloid cells and lymphoid cells. Specifically contemplated immune cells include macrophage, myeloid, neutrophil, fibroblast, basophil, mast, eosinophil, B, natural killer (NK), T, and dendritic cells.

(Immune) Checkpoint Inhibitor: Checkpoint inhibitor therapy is a form of cancer immunotherapy that targets immune “checkpoints”, which are key regulators of the immune system. When stimulated, these checkpoints can dampen the immune response to an immunologic stimulus. Some cancers can stimulate immune checkpoint target(s) and thereby evade native immune response. Checkpoint therapy can block inhibitory checkpoints, thereby restoring immune system function (Pardoll, Nat Reviews 12(4):252-264, 2012). Drugs or drug candidates that inhibit/block the inhibitory checkpoint molecules are sometimes known as checkpoint inhibitors; this idea is often referred to as immune checkpoint blockade, or simply checkpoint blockade.

Three well known immune checkpoint targets are: PD-1, a transmembrane programmed cell death 1 protein (also called PDCD1 and CD279), which interacts with PD-L1 (PD-1 ligand 1, also called CD274); PD-L1, a cell surface protein binds to PD-1 on an immune cell surface, thereby inhibiting immune cell activity; and CTLA4 (cytotoxic T-lymphocyte-associated protein 4; aka CD152), a protein receptor that downregulates immune responses. Additional representative inhibitory checkpoint targets include 2B4, A2AR, B7-H3, B7-H4, BTLA, CTLA-4, IDO, KIR, LAIRI, NOX2, PD-1, PD-L1, VISTA, and SIGLEC7. LAG-3, TIM-3, and TIGIT are also targets; see, e.g., Anderson et al., Immunity. 44(5):989-1004, 2016.

The first anti-cancer drug targeting an immune checkpoint approved in the United States was ipilimumab (Yervoy™), a CTLA4 blocker. Additional approved checkpoint inhibitors that target PD-1 include: nivolumab (Opdivo™), perbrolizumab (Keytruda™), and cemiplimab (Libatyo™); and that target PD-L1 include: atezolizumab (Tecentriq™), Avelumab (Barvencio™), and durvalumab (Imfinzi™).

Immune function: “Immune function” refers to the appropriate or correct proliferation and activity of immune cells in the body. Immune function protects an organism from foreign invasion and insults. Methods of measuring immune function include counting subpopulations of immune cells, plaque forming, chemotaxis, random migration, superoxide anion release, concentration of ATP in circulating CD4+ cells following in vitro stimulation with phytohemagglutinin, and release of fluorescent dye from target cells assays.

Improve: A qualitative or quantitative difference from a reference, resulting in a better outcome.

Improved immune therapy product: “Improved immune therapy product” refers to a product including a “gene product”, “expression product”, or “cell product” that functions (e.g., in a therapeutic treatment) to improve immune function.

Improved efficacy of Immune checkpoint blockade therapy: “Immune checkpoint blockade therapy” removes one or more inhibitory signals of immune cell activation, which enables the immune cells to overcome regulatory mechanisms and mount an effective immune response. The checkpoint blockade therapy is usually effected by using a checkpoint inhibitor, particularly where the target of the therapy is an inhibitory checkpoint molecule. Targets for immune checkpoint inhibition include 2B4 (natural killer cell receptor 2B4), A2AR (Adenosine A2A receptor), B7-H3 (B7 Homolog 3; aka CD276), B7-H4 (B7 Homolog 4; VTCN1), BTLA (B- and T-lymphocyte attenuator), CTLA-4 (cytotoxic T lymphocyte antigen-4), IDO (Indoleamine 2,3-dioxygenase), KIR (Killer-cell Immunoglobulin-like Receptor), LAIRI (leukocyte-associated immunoglobulin-like receptor 1), LAG3 (lymphocyte-activation protein 3), NOX2 (NADPH oxidase 2), PD-1 (Programmed Death 1 receptor), SIGLEC7 (Sialic acid-binding immunoglobulin-type lectin 7; aka CD328), TIM-3 (T cell immunoglobulin mucin-3), TIGIT (T cell immunoreceptor with Ig and ITIM domains), and VISTA (V-domain Ig suppressor of T cell activation)). “Improved efficacy” refers to the increase in a desired results, so the “improved efficacy of immune checkpoint blockade therapy” refers to an increase in a therapy's ability to remove inhibitory signals of immune cell activation thus increasing the immune response.

Inhibition: As used herein, the term “inhibition,” when used to describe an effect on a gene, refers to a measurable decrease in the cellular level of mRNA transcribed from the gene and/or peptide, polypeptide, or protein product of the gene. In some examples, expression of a gene may be inhibited such that expression is substantially or essentially eliminated, and the use of the term “inhibit” herein specifically includes both a reduction in gene expression that leads to a measurable characteristic in the organism, in some examples to “substantially eliminate” or “essentially eliminate” (used interchangeably herein) expression, such that the amount of the gene's activity is undetectable or below a significant amount. “Specific inhibition” refers to the inhibition of a target gene or family of genes without consequently affecting expression of other unrelated genes in the cell wherein the specific inhibition is being accomplished.

Inhibition can also refer to reduction or limitation of an activity or function, such as inhibition of an enzyme or transcription factor, or more generally reduction or limitation of a response. One exemplary response is an immune response, such as an immunotherapy response (that is an immune response to an applied immunotherapy).

Inhibition of an immunotherapy response: Refers to a reduction or limitation of the function of an immune response to an applied immunotherapy.

Isolated: An “isolated” biological component (such as a polynucleotide, polypeptide, or small molecules (e.g., hormones)) has been substantially separated, produced apart from, or purified away from other biological components in the cell of the organism in which the component originated or was made or naturally occurs (i.e., other chromosomal and extra-chromosomal DNA and RNA, and proteins), while effecting a chemical or functional change in the component (e.g., a nucleic acid may be isolated from a chromosome by breaking chemical bonds connecting the nucleic acid to the remaining DNA in the chromosome; or a chemical compound may be converted to a purified form that is effective or more effective for some use(s) because it is removed from the presence of other components, which may be viewed as contaminants). Polynucleotides and small molecules that have been isolated specifically include nucleic acid molecules and cannabinoids purified by standard purification methods. The term also embraces biological components (such as nucleic acid molecules and cannabinoids) prepared by recombinant expression or production in a host organism or host cell, as well as chemically-synthesized versions, including when they are substantially separated or purified away from other biological components in that product milieu.

Label: Any substance capable of aiding a machine, detector, sensor, device, column, or enhanced or unenhanced human eye from differentiating a labeled composition from an unlabeled composition. Labels may be used for any of a number of purposes and one skilled in the art will understand how to match the proper label with the proper purpose.

Examples of uses of labels include purification of biomolecules, identification of biomolecules, detection of the presence of biomolecules, detection of protein folding, and localization of biomolecules within a cell, tissue, or organism. Examples of labels include: radioactive isotopes or chelates thereof; dyes (fluorescent or non-fluorescent), stains, enzymes, nonradioactive metals, magnets, protein tags, fluorescent proteins, any antibody epitope, any specific example of any of these; any combination between any of these, or any label now known or yet to be disclosed. A label may be covalently attached to a biomolecule or bound through hydrogen bonding, Van Der Waals or other forces. A label may be covalently or otherwise bound to the N-terminus, the C-terminus or any amino acid of a polypeptide or the 5′ end, the 3′ end or any nucleic acid residue in the case of a polynucleotide.

One particular example of a label is a small molecule fluorescent dye. Such a label can be conjugated to an antibody such as an antibody that binds a macrophage or tumor cell marker. One of skill in the art would be able to identify and select any appropriate fluorescent dye or combination of fluorescent dyes for use in the disclosed methods.

Another particular example of a label is a protein tag. A protein tag includes a sequence of one or more amino acids that may be used as a label as discussed above, particularly for use in protein purification. In some examples, the protein tag is covalently bound to the polypeptide. It may be covalently bound to the N-terminal amino acid of a polypeptide, the C-terminal amino acid of a polypeptide or any other amino acid of the polypeptide. Often, the protein tag is encoded by a polynucleotide sequence that is immediately 5′ of a nucleic acid sequence coding for the polypeptide such that the protein tag is in the same reading frame as the nucleic acid sequence encoding the polypeptide. Protein tags may be used for all of the same purposes as labels listed above and are well known in the art. Examples of protein tags include chitin binding protein (CBP), maltose binding protein (MBP), glutathione-S-transferase (GST), poly-histidine (His), thioredoxin (TRX), FLAG™, V5, c-Myc, HA-tag, and so forth. A His-tag facilitates purification and binding to on metal matrices, including nickel matrices, including nickel matrices bound to solid substrates such as agarose plates or beads, glass plates or beads, or polystyrene or other plastic plates or beads. Other protein tags include BCCP, calmodulin, Nus, Thioredoxin, Streptavidin, SBP, and Ty, or any other combination of one or more amino acids that can work as a label described above.

Another particular example of a label is biotin. Biotin is a natural compound that tightly binds proteins such as avidin or streptavidin. A compound labeled with biotin is said to be ‘biotinylated’. Biotinylated compounds can be detected with avidin or streptavidin when that avidin or streptavidin is conjugated another label (such as a fluorescent, enzymatic, radioactive or other label).

Leukocyte: A heterogeneous group of nucleated blood cell types, and excludes erythrocytes and platelets. Leukocytes can be divided into two general groups: polymorphonucleocytes, which include neutrophils, eosinophils, and basophils, and mononucleocytes, which include lymphocytes and monocytes. Polymorphonucleocytes contain many cytoplasmic granules and a multilobed nucleus and include the following: neutrophils, which are generally amoeboid in shape, phagocytic, and stain with both basic and acidic dyes, and eosinophils and basophils, which contain cytoplasmic granules that stain with acidic dyes and with basic dyes, respectively.

Locus: As used herein, the term “locus” refers to a position on the genome that corresponds to a gene, a marker thereof, or a measurable characteristic (e.g., a trait). A locus may be unambiguously defined by an oligonucleotide (e.g., a probe) that specifically hybridizes to a polynucleotide at the locus.

Lymphocyte: A mononuclear leukocyte that is involved in the mounting of an immune response. In general, lymphocytes include B lymphocytes, T lymphocytes, and NK cells.

Metastasis: The process through which a tumor in a primary site releases single tumor cells that seed other distant organ sites. This phase of tumorigenesis is most fatal (90% mortality). Metastatic disease or metastasis refers to cancer cells that have left the original tumor site and migrate to other parts of the body for example via the bloodstream or lymph system. The “pathology” of cancer includes all phenomena that compromise the well-being of the subject. This includes, abnormal or uncontrollable cell growth, metastasis, interference with the normal functioning of neighboring cells, release of cytokines or other secretory products at abnormal levels, suppression, or aggravation of inflammatory or immunological response, neoplasia, premalignancy, malignancy, invasion of surrounding or distant tissues or organs, such as lymph nodes, etc.

Monocyte: A CD14+ and CD34− peripheral blood mononuclear cell (PBMC), which is generally capable of differentiating into a macrophage and/or dendritic cell upon activation by one or more foreign substances, such as, a microbial product. In particular, a monocyte may express elevated levels of the CD14 surface antigen marker, and may express at least one biomarker selected from CD64, CD93, CD180, CD328 (also known as sialic acid-binding Ig-like lectin 7 or Siglec7), and CD329 (sialic acid-binding Ig-like lectin 9 or Siglec9), as well as the peanut agglutinin protein (PNA).

Nucleic acid molecule: As used herein, the term “nucleic acid molecule” refers to a polymeric form of nucleotides, which includes in specific examples both or either of sense and antisense strands of RNA, cDNA, genomic DNA, and synthetic forms and mixed polymers of the foregoing. The term includes single- and double-stranded forms of DNA and RNA. A nucleic acid molecule can include either or both naturally occurring and modified nucleotides linked together by naturally occurring and/or non-naturally occurring nucleotide linkages. A nucleotide may be a ribonucleotide, deoxyribonucleotide, or modified form of either. A “polynucleotide” refers to a physical contiguous nucleotide polymer, such as may be included in a larger nucleic acid molecule. A nucleic acid molecule is usually at least 10 bases in length, unless otherwise specified. By convention, the nucleotide sequence of a nucleic acid molecule is read from the 5′ to the 3′ end of the molecule. The “complement” of a nucleic acid molecule refers to a polynucleotide having nucleobases that may form base pairs with the nucleobases of the nucleic acid molecule (i.e., A-T/U, and G-C).

Some embodiments include nucleic acids including a template DNA that is transcribed into an RNA molecule that includes a polyribonucleotide that hybridizes to a mRNA molecule. In some examples, the template DNA is the complement of the polynucleotide transcribed into the mRNA molecule, present in the 5′ to 3′ orientation, such that RNA polymerase (which transcribes DNA in the 5′ to 3′ direction) will transcribe the polyribonucleotide from the complement that can hybridize to the mRNA molecule. Unless explicitly stated otherwise, or it is clear to be otherwise from the context, the term “complement” therefore refers to a polynucleotide having nucleobases, from 5′ to 3′, that may form base pairs with the nucleobases of a reference nucleic acid. In some examples, the template DNA is the reverse complement of the polynucleotide transcribed into the mRNA molecule. Thus, unless it is explicitly stated to be otherwise (or it is clear to be otherwise from the context), the “reverse complement” of a polynucleotide refers to the complement in reverse orientation. The foregoing is demonstrated in the following illustration:
ATGATGATG polynucleotide
TACTACTAC “complement” of the polynucleotide
CATCATCAT “reverse complement” of the polynucleotide

As used herein, two polynucleotides are said to exhibit “complete complementarity” when every nucleotide of a polynucleotide read in the 5′ to 3′ direction is complementary to every nucleotide of the other polynucleotide when read in the 5′ to 3′ direction. Similarly, a polynucleotide that is completely reverse complementary to a reference polynucleotide will exhibit a nucleotide sequence where every nucleotide of the polynucleotide read in the 5′ to 3′ direction is complementary to every nucleotide of the reference polynucleotide when read in the 3′ to 5′ direction. These terms and descriptions are recognized in the art and are understood by those of ordinary skill in the art.

Some embodiments of the disclosure include hairpin RNA (hpRNA)-forming RNA molecules. In these hpRNA molecules, both a polyribonucleotide that is substantially identical to the complement or reverse complement of a target ribonucleotide sequence in the target mRNA, and a polyribonucleotide that is substantially the reverse complement thereof, may be found in the same molecule, such that the single-stranded transcribed RNA molecule may “fold over” and hybridize to itself over a region including both polyribonucleotides (i.e., in a “stem structure” of the hpRNA).

“Nucleic acid molecules” include all polynucleotides, for example: single- and double-stranded forms of DNA; single-stranded forms of RNA; and double-stranded forms of RNA (dsRNA). The term “nucleotide sequence” or “nucleic acid sequence” refers to both the sense and antisense strands of a nucleic acid as either individual single strands or in the duplex. The term “ribonucleic acid” (RNA) is inclusive of iRNA (inhibitory RNA), dsRNA (double stranded RNA), siRNA (small interfering RNA), shRNA (small hairpin RNA), mRNA (messenger RNA), miRNA (micro-RNA), hpRNA (hairpin RNA), tRNA (transfer RNAs, whether charged or discharged with a corresponding acylated amino acid), and cRNA (complementary RNA). The term “deoxyribonucleic acid” (DNA) is inclusive of cDNA, gDNA, and DNA-RNA hybrids. The terms “polynucleotide” and “nucleic acid,” and “fragments” thereof will be understood by those in the art as a term that includes both gDNAs, ribosomal RNAs, transfer RNAs, messenger RNAs, operons, and smaller engineered polynucleotides that encode or may be adapted to encode, peptides, polypeptides, or proteins.

Oligonucleotide: An oligonucleotide is a short nucleic acid polymer (a short nucleic acid molecule). Oligonucleotides may be formed by cleavage of longer nucleic acid segments, or by polymerizing individual nucleotide precursors. Automated synthesizers allow the synthesis of oligonucleotides up to several hundred bases in length. Because oligonucleotides may bind to a complementary nucleic acid, they may be used as probes for detecting DNA or RNA. Oligonucleotides composed of DNA (oligodeoxyribonucleotides) may be used in PCR, a technique for the amplification of DNAs. In PCR, the oligonucleotide is typically referred to as a “primer,” which allows a DNA polymerase to extend the oligonucleotide and replicate the complementary strand. Oligonucleotides may also be used in embodiments herein as a probe, either to detect specific polynucleotides or polyribonucleotides as part of an in vitro process, or to detect polynucleotides or polyribonucleotides in a sample.

A nucleic acid molecule may include either or both naturally occurring and modified nucleotides linked together by naturally occurring and/or non-naturally occurring nucleotide linkages. Nucleic acid molecules may be modified chemically or biochemically, or may contain non-natural or derivatized nucleotide bases, as will be readily appreciated by those of skill in the art. Such modifications include, for example, labels, methylation, substitution of one or more of the naturally occurring nucleotides with an analog, internucleotide modifications (e.g., uncharged linkages: for example, methyl phosphonates, phosphotriesters, phosphoramidates, carbamates, etc.; charged linkages: for example, phosphorothioates, phosphorodithioates, etc.; pendent moieties: for example, peptides; intercalators: for example, acridine, psoralen, etc.; chelators; alkylators; and modified linkages; for example, alpha anomeric nucleic acids, etc.). The term “nucleic acid molecule” also includes any topological conformation, including single-stranded, double-stranded, partially duplexed, triplexed, hairpinned, circular, and padlocked conformations.

As used herein with respect to DNA, the term “coding polynucleotide,” “structural polynucleotide,” or “structural nucleic acid molecule” refers to a polynucleotide that is ultimately transcribed into an RNA; for example, when placed under the control of appropriate regulatory elements. The boundaries of a coding polynucleotide are determined by a translation start codon at the 5′-terminus and a translation stop codon at the 3′-terminus. Coding polynucleotides include, but are not limited to, gDNA, cDNA, ESTs, and recombinant polynucleotides. As used herein, “transcribed non-coding polyribonucleotide” refers to segments of mRNA molecules such as 5′UTR, 3′UTR, and intron segments that are not translated into a polypeptide. For example, a transcribed non-coding polyribonucleotide may be a polyribonucleotide that natively exists as an intragenic “spacer” in an RNA molecule.

Operably linked: A first polynucleotide is operably linked with a second polynucleotide when the first polynucleotide is in a functional relationship with the second polynucleotide. When recombinantly produced, operably linked polynucleotides are generally contiguous, and, where necessary to join two coding regions, in the same reading frame (e.g., in a translationally fused ORF). However, polynucleotides need not be contiguous to be operably linked. The term, “operably linked,” when used in reference to a regulatory genetic element and a polynucleotide, means that the regulatory element affects the expression of the linked polynucleotide. “Regulatory elements,” “control elements,” or “regulatory sequences” refer to polynucleotides that influence the timing and level/amount of transcription (or RNA processing or stability) of the operably linked polynucleotide. Regulatory sequences include, for example and without limitation promoters, translation leaders, introns, enhancers, stem-loop structures, repressor binding sequences, termination sequences, and polyadenylation recognition sequences. Particular regulatory elements may be located upstream and/or downstream of a polynucleotide operably linked thereto. Also, particular regulatory elements operably linked to a polynucleotide may be located on the associated complementary strand of a double-stranded nucleic acid molecule.

Polypeptide: Any chain of amino acids, regardless of length or posttranslational modification (such as glycosylation, methylation, ubiquitination, phosphorylation, or the like). The term polypeptide is used interchangeably with peptide or protein, and is used to refer to a polymer of amino acid residues. The term residue refers to an amino acid or amino acid mimetic incorporated in a polypeptide by an amide bond or amide bond mimetic.

Purification: Generally, the removal of contaminants or impurities (that is, undesired component(s) or adulterants) from a mixed substance or milieu. Purification of a cell or cell type can be achieved by any method known in the art including by use of methods that involve the use of labeled antibodies that bind cell surface antigens such as fluorescence activated cell sorting, sorting through the use of magnetic beads, or on purification columns. Purification does not require absolute purity (that is the purified cells are exactly 100% cells of the desired type). Instead, a purified population of cells can include at least 60%, 70%, 80%, 90%, 95%, 98%, 99% 99.9%, or 99.99% cells of the desired type.

Sequence Identity: The term “sequence identity” or “identity,” as used herein in the context of two nucleotide sequences, refers to the residues in the two sequences that are the same when aligned for maximum correspondence over a specified comparison window. The term “percentage of sequence identity” may refer to the value determined by comparing two optimally aligned nucleotide sequences over a comparison window, wherein the portion of the nucleotide sequence in the comparison window may include additions or deletions (i.e., gaps) as compared to the reference sequence (which does not include additions or deletions) for optimal alignment of the two sequences. The percentage is calculated by determining the number of positions at which the identical nucleotide or amino acid residue occurs in both sequences to yield the number of matched positions, dividing the number of matched positions by the total number of positions in the comparison window, and multiplying the result by 100 to yield the percentage of sequence identity.

Methods for aligning sequences for comparison are well-known in the art. Various programs and alignment algorithms are described in, for example: Smith and Waterman, Adv. Appl. Math. 2:482, 1981; Needleman and Wunsch, J. Mol. Biol. 48:443, 1970; Pearson and Lipman, Proc. Natl. Acad. Sci. U.S.A. 85:2444, 1988; Higgins and Sharp, Gene 73:237-44, 1988; Higgins and Sharp, CABIOS 5:151-3, 1989; Corpet et al., Nucleic Acids Res. 16:10881-90, 1988; Huang et al., Comp. Appl. Biosci. 8:155-65, 1992; Pearson et al., Methods Mol. Biol. 24:307-31, 1994; Tatiana et al., FEMS Microbiol. Lett. 174:247-50, 1999. A detailed consideration of sequence alignment methods and homology calculations can be found in, e.g., Altschul et al., J. Mol. Biol. 215:403-10, 1990.

The National Center for Biotechnology Information (NCBI) Basic Local Alignment Search Tool (BLAST™; Altschul et al. (1990)) is available from several sources, including the National Center for Biotechnology Information (Bethesda, MD), and on the internet, for use in connection with several sequence analysis programs. A description of how to determine sequence identity using this program is available on the internet under the “help” section for BLAST™. For comparisons of nucleic acid sequences, the “Blast 2 sequences” function of the BLAST™ (Blastn) program may be employed using the default parameters. Nucleotide sequences with even greater similarity to the reference sequences will show increasing percentage identity when assessed by this method.

Specifically hybridizable/Specifically complementary: As used herein, the terms “specifically hybridizable,” “specifically complementary,” and “specifically reverse complementary” indicate a sufficient degree of complementarity or reverse complementarity such that stable and specific binding occurs between a polyribonucleotide and a nucleic acid molecule including a target polyribonucleotide. As is well-known in the art, a polyribonucleotide need not be 100% complementary to its target polyribonucleotide to be specifically hybridizable. In RNAi applications using hpRNAs, the lower free energy required for intramolecular hybridization (as compared to intermolecular hybridization) facilitates the hybridization of partially complementary or reverse complementary primary transcripts (for example, transcripts including loop-forming sequences and non-hybridizing sequences in a stem between siRNA sequences).

As used herein, the term “identical,” “substantial identity,” “substantially homologous,” or “substantial homology,” with regard to a reference polyribonucleotide, refers to a polyribonucleotide having contiguous nucleotides that hybridize to a polyribonucleotide or oligonucleotide consisting of the nucleotide sequence of the reference polyribonucleotide. For example, an siRNA consisting of the polyribonucleotide encoded by any of SEQ ID NOs: 17, 19, 24, 28, 43, and 80 is substantially homologous or substantially identical to a reference polyribonucleotide if the siRNA hybridizes to the reference polyribonucleotide. Substantially identical polyribonucleotides herein (e.g., siRNAs) share at least 80% sequence identity.

In examples herein, substantially identical polyribonucleotides have between 80% and 100% sequence identity. In particular examples, substantially identical polyribonucleotides have between 80% and 100% sequence identity. In other examples, substantially identical polyribonucleotides have between 85% and 100% sequence identity. In other examples, substantially identical polyribonucleotides have between 86% and 100% sequence identity. In other examples, substantially identical polyribonucleotides have between 87% and 100% sequence identity. In other examples, substantially identical polyribonucleotides have between 88% and 100% sequence identity. In other examples, substantially identical polyribonucleotides have between 89% and 100% sequence identity. In other examples, substantially identical polyribonucleotides have between 90% and 100% sequence identity. In other examples, substantially identical polyribonucleotides have between 91% and 100% sequence identity. In other examples, substantially identical polyribonucleotides have between 92% and 100% sequence identity. In other examples, substantially identical polyribonucleotides have between 93% and 100% sequence identity. In other examples, substantially identical polyribonucleotides have between 94% and 100% sequence identity. In other examples, substantially identical polyribonucleotides have between 95% and 100% sequence identity. In yet other examples, substantially identical polyribonucleotides have between 96% and 100% sequence identity.

The property of substantial identity is closely related to specific hybridization. For example, a polyribonucleotide is specifically hybridizable when there is a sufficient degree of complementarity or reverse complementarity to avoid non-specific binding of the polyribonucleotide to non-target polyribonucleotides.

Subject (Patient): A living multicellular vertebrate organism, a category that includes, for example, mammals and birds. A mammal includes both human and non-human mammals, such as mice. In some examples, a subject is a patient, such as a patient diagnosed with cancer. In other examples, a subject is a patient yet to be diagnosed.

Therapeutically effective amount: (or Pharmaceutically effective amount) An amount that is sufficient to effect treatment or prophylaxis, as illustrated herein, when administered to a subject (e.g., a mammal, such as a human) in need of such treatment. The therapeutically or pharmaceutically effective amount will vary depending upon the subject and disease condition being treated, the weight and age of the subject, the severity of the disease condition, the manner of administration and the like, which can readily be determined by one of ordinary skill in the art. For example, a “therapeutically effective amount” or a “pharmaceutically effective amount” of a compound (such as an inhibitor of AR function, or a siRNA or other moderator of AR level or inhibitor of AR expression, or a checkpoint inhibitor), or a pharmaceutically acceptable salt or co-crystal thereof, is an amount sufficient to modulate expression or activity of the target inhibited by that agent (such as, for instance, androgen receptor level or activity), and thereby treat a subject (e.g., a human) exhibiting or suffering an indication, or to ameliorate or alleviate the existing symptoms of the indication. For example, a therapeutically or pharmaceutically effective amount may be an amount sufficient to decrease a symptom of a disease or condition responsive to inhibition of AR activity.

Tumor: Neoplastic cell growth and proliferation, whether malignant or benign, including all pre-cancerous and cancerous cells and tissues. Tumor markers include polynucleotides and polypeptides expressed by tumors to a greater extent than they are expressed by non-tumor cells, including cell surface or cytoplasmic or nuclear tumor antigens.

Examples of types of tumors include acute lymphoblastic leukemia; acute myeloid leukemia; adrenocortical carcinoma; AIDS-related cancers; AIDS-related lymphoma; anal cancer; appendix cancer; astrocytoma cerebellar or cerebral; basal cell carcinoma; extrahepatic bile duct cancer; bladder cancer; bone cancer, osteosarcoma/malignant fibrous histiocytoma; brainstem glioma; brain tumor; brain tumor, cerebellar astrocytoma; brain tumor, cerebral astrocytoma/malignant glioma; brain tumor, ependymoma; brain tumor, medulloblastoma; brain tumor, supratentorial primitive neuroectodermal tumors; brain tumor, visual pathway and hypothalamic glioma; breast cancer; bronchial adenomas/carcinoids; Burkitt lymphoma; carcinoid tumor; carcinoid tumor, gastrointestinal; carcinoma of unknown primary; central nervous system lymphoma, primary; cerebellar astrocytoma; cerebral astrocytoma/malignant glioma; cervical cancer; childhood cancers; chronic lymphocytic leukemia; chronic myelogenous leukemia; chronic myeloproliferative disorders; colon cancer; cutaneous T-cell lymphoma; Desmoplastic small round cell tumor; endometrial cancer; ependymoma; esophageal cancer; Ewing's sarcoma in the Ewing family of tumors; extracranial germ cell tumor; extragonadal germ cell tumor; extrahepatic bile duct cancer; eye cancer, intraocular melanoma; eye cancer, retinoblastoma; gallbladder cancer; gastric (stomach) cancer; gastrointestinal carcinoid tumor; gastrointestinal stromal tumor (GIST); germ cell tumor: extracranial, extragonadal, or ovarian; gestational trophoblastic tumor; glioma of the brain stem; glioma cerebral astrocytoma; glioma visual pathway and hypothalamic; gastric carcinoid; hairy cell leukemia; head and neck cancer; heart cancer; hepatocellular (liver) cancer; Hodgkin lymphoma; hypopharyngeal cancer; hypothalamic and visual pathway glioma; intraocular melanoma; islet cell carcinoma (endocrine pancreas); Kaposi sarcoma; kidney cancer (renal cell cancer); laryngeal cancer; leukemias; acute lymphoblastic leukemia (also called acute lymphocytic leukemia); acute myeloid leukemia (also called acute myelogenous leukemia); leukemia, chronic lymphocytic (also called chronic lymphocytic leukemia); chronic myelogenous leukemia (also called chronic myeloid leukemia); hairy cell leukemia; lip and oral cavity cancer; liver cancer (primary); non-small cell lung cancer; small cell lung cancer; lymphomas; AIDS-related lymphoma; Burkitt lymphoma; cutaneous t-cell lymphoma; Hodgkin lymphoma; lymphomas, non-Hodgkin lymphoma (an old classification of all lymphomas except Hodgkin's); primary central nervous system lymphoma; Marcus whittle, deadly disease; malignant fibrous histiocytoma of bone/osteosarcoma; medulloblastoma; melanoma; intraocular (eye) melanoma; Merkel cell carcinoma; mesothelioma; metastatic squamous neck cancer with occult primary; mouth cancer; multiple endocrine neoplasia syndrome; multiple myeloma/plasma cell neoplasm; mycosis fungoides; myelodysplastic syndromes; myelodysplastic/myeloproliferative diseases; myelogenous leukemia, chronic; myeloid leukemia acute; myeloid leukemia acute; myeloma, multiple (cancer of the bone-marrow); chronic myeloproliferative disorders; nasal cavity and paranasal sinus cancer; nasopharyngeal carcinoma; neuroblastoma; non-Hodgkin lymphoma; non-small cell lung cancer; oral cancer; oropharyngeal cancer; osteosarcoma/malignant fibrous histiocytoma of bone; ovarian cancer; ovarian epithelial cancer (surface epithelial-stromal tumor); ovarian germ cell tumor; ovarian low malignant potential tumor; pancreatic cancer; islet cell pancreatic cancer; paranasal sinus and nasal cavity cancer; parathyroid cancer; penile cancer; pharyngeal cancer; pheochromocytoma; pineal astrocytoma; pineal germinoma; pineoblastoma and supratentorial primitive neuroectodermal tumors; pituitary adenoma; plasma cell neoplasia/multiple myeloma; pleuropulmonary blastoma; primary central nervous system lymphoma; prostate cancer; rectal cancer; renal cell carcinoma (kidney cancer); renal pelvis and ureter, transitional cell cancer; retinoblastoma; rhabdomyosarcoma; salivary gland cancer; sarcoma, Ewing family of tumors; Kaposi sarcoma; soft tissue sarcoma; uterine sarcoma; Sezary syndrome; skin cancer (nonmelanoma); skin cancer (melanoma); skin carcinoma, Merkel cell; small cell lung cancer; small intestine cancer; soft tissue sarcoma; squamous cell carcinoma; squamous neck cancer with occult primary, metastatic; stomach cancer; supratentorial primitive neuroectodermal tumor; cutaneous T-Cell lymphoma; testicular cancer; throat cancer; thymoma; thymoma and thymic carcinoma; thyroid cancer; thyroid cancer; transitional cell cancer of the renal pelvis and ureter; gestational trophoblastic tumor; ureter and renal pelvis, transitional cell cancer; urethral cancer; uterine cancer, endometrial; uterine sarcoma; vaginal cancer; visual pathway and hypothalamic glioma; vulvar cancer; Waldenstrom macroglobulinemia; and Wilms tumor.

### Reduction of Androgen Receptor Level to Enhance Anti-Cancer Therapy

Described herein are methods, compositions, and systems that enhance anti-cancer therapy(s) through reduction of androgen receptor level and/or activity. By limiting androgen receptor activity, multiple cell products that have enhanced anti-cancer function (and proliferation) can be generated for cancer immunotherapy. Some of the individual populations include:
Reduction in T cell intrinsic androgen receptor level and/or function to improve T cell function: allogenic T-cell therapy, adoptive cell therapy, autologous T cell therapy, CAR-T; administration of CAR-T therapy either modified to lack AR or delivered in conjunction with pharmacologic inhibition of AR activity (i.e. with an AR small molecule inhibitor or degrader or shRNA);
Reduction in T cell intrinsic androgen receptor level and/or function to reduce regulatory T cell suppression such as immunotherapy provided with AR targeted therapy to reduce regulatory T cell function as AR enforces regulatory T cell stability;
Reduction in myeloid and/or macrophage intrinsic androgen receptor levels to improve engineered macrophage therapy such as AR deletion of a myeloid/macrophage cell therapy product or pharmacologic inhibition of AR (see above) with myeloid/macrophage therapy for the treatment of cancer;
Reduction in dendritic cell intrinsic androgen receptor level and/or function to improve antigen presentation; as is in the case of dendritic cell vaccines for the treatment of cancer. Dendritic cell therapy projects could be engineered to reduce intrinsic AR expression and/or delivered with pharmacologic inhibition of AR during treatment. Further, it is recognized that there are sex differences in effectiveness of DC vaccines for cancer. By way of example, this was demonstrated in Storkus et al. (J. Immunother Cancer, 9:e003675, 2021; doi:10.1136/jitc-2021-003675). The authorize found that the Immune Response Rate (80% vs 25%) and the Objective Response Rate (60% vs 12.5%) were greater for female versus male patients. It is therefore proposed that reduction in intrinsic AR level and/or function in DC of male patients (as taught herein) can be used to increase the effectiveness of treatments (such as antitumor treatment) in those patients.

Reduction in natural killer cell (NK) intrinsic androgen receptor level and/or function to improve cell killing; NK cell therapy product could be genetically modified to express less AR prior to infusion and/or delivered with pharmacologic inhibition of AR to improve the performance of NK cell therapy for the purposes of cancer therapy.

Reduction in B cell intrinsic androgen receptor and/or function improves antibody maturation, and antibody production in that deletion of AR and/or treatment with pharmacologic inhibition of AR could promote B cell maturation, antibody class switching and somatic hypermutation, and the survival of B cells and plasmablasts.

### Targeted Gene Editing/Disruption

In particular embodiments, gene deletion of AR is mediated by a gene editing system such as Clustered Regularly Interspaced Short Palindromic Repeats/CRISPR-associated (CRISPR/Cas), transcription activator-like effector nucleases (TALENs), zinc-finger nucleases (ZFNs), and/or meganucleases. In particular embodiments, a gene (such as a gene encoding androgen receptor, such as the exemplary human gene publicly available as GenBank Accession No. AH002624.2, which encodes exemplary AR protein GenBank. Accession No. AAA51886). In particular embodiments, a gene is deleted by introducing one or more mutations that disable the function of a protein encoded by the gene(s). In particular embodiments, a gene is partially or completely removed from the genome of a target immune cell or population of immune cells, either in vivo or ex vivo.

The widely popular CRISPR/Cas9 system is one method by which targeted disruption is performed. The CRISPR nuclease system is a prokaryotic immune system that confers resistance to foreign genetic elements such as plasmids and phages and provides a form of acquired immunity. CRISPRs are DNA loci containing short repetitions of base sequences. In the context of a prokaryotic immune system, each repetition is followed by short segments of spacer DNA belonging to foreign genetic elements that the prokaryote was exposed to. This CRISPR array of repeats interspersed with spacers can be transcribed into RNA. The RNA can be processed to a mature form and associate with a Cas (CRISPR-associated) nuclease. A CRISPR-Cas system including an RNA having a sequence that can hybridize to the foreign genetic elements and Cas nuclease can then recognize and cut these exogenous genetic elements in the genome.

A single Cas enzyme can be programmed by a gRNA molecule to site-specifically cleave a specific target nucleic acid. Cas9 is an exemplary Type II CRISPR Cas protein. Cas9 includes two distinct endonuclease domains (HNH and RuvC/RNase H-like domains), one for each strand of the target nucleic acid. RuvC and HNH together produce double-stranded breaks (DSBs); separately each domain can produce single-stranded breaks. Base-pairing between the gRNA and target nucleic acid causes double-stranded breaks (DSBs) due to the endonuclease activity of Cas9. Binding specificity is determined by both gRNA-target nucleic acid base pairing and the PAM juxtaposed to the DNA complementary region. In particular embodiments, the CRISPR system only requires a minimal set of two molecules—the Cas protein and the gRNA.

A large number of Cas9 orthologs are known in the art (Fonfara et al., NAR, 42:2577-2590, 2014; Chylinski et al., NAR, 42:6091-6105, 2014; Esvelt et al., Nature Methods, 10:1116-1121, 2013). A number of orthogonal Cas9 proteins have been identified including Cas9 proteins from Neisseria meningitidis, Streptococcus thermophilus and Staphylococcus aureus. Other Class 2 Cas proteins that can be used include Cas12a (Cpf1), Cas13a (C2c2), and Cas13B (C2c6). The Cpf1 nuclease particularly can provide added flexibility in target site selection by means of a short, three base pair recognition sequence (TTN), known as the protospacer-adjacent motif or PAM. Cpf1's cut site is at least 18 bp away from the PAM sequence, thus the enzyme can repeatedly cut a specified locus after indel (insertion and deletion) formation. Exemplary engineered Cpf1s are described in US 2018/0030425, US 2016/0208243, WO/2017/184768 and Zetsche et al., Cell 163: 759-771, 2015; and single gRNAs in Jinek et al., Science 337:816-821, 2012; Jinek et al., eLife 2:e00471, 2013; Segal, eLife 2:e00563, 2013.

In particular embodiments, polynucleotide sequences encoding mutant forms of Cas9 nuclease can be used in genetic constructs of the disclosure. For example, a Sniper Cas9, a variant of Cas9 with optimized specificity (minimal off-target effects) and retained on-target activity can be used (Lee et al., J Vis Exp. (144), 2019; Lee et al., Nat Commun. 9(1):3048, 2018; WO 2017/217768). As another example, a mutant Cas9 nuclease containing a D10A amino acid substitution can be used. This mutant Cas9 has lost double-stranded nuclease activity present in the wild type Cas9 but retains partial function as a single-stranded nickase. This mutant Cas9 generates a break in the complementary strand of DNA rather than both strands. This allows repair of the DNA template using a high-fidelity pathway rather than non-homologous end joining (NHEJ). The higher fidelity pathway prevents formation of insertions/deletions at the targeted locus while maintaining ability to undergo homologous recombination (Cong et al., Science 339(6121):819-823, 2013). Paired nicking has been shown to reduce off-target activity by 50- to 1,500-fold in cell lines (Ran et al., Cell 154(6):1380-1389, 2013).

In particular embodiments, a Cas protein can include one or more degrons to self-inactivate the Cas protein by accelerating degradation of expressed Cas protein. A degron can include a portion of a polypeptide that is important in regulation of protein degradation. In particular embodiments, a degron includes short amino acid sequences, structural motifs, and/or exposed amino acids (e.g., a lysine or arginine) located anywhere in a protein. In particular embodiments, a degron can be ubiquitin-dependent or ubiquitin-independent.

In particular embodiments, a Cas protein can be fused to a heterologous polypeptide that provides for subcellular localization. Such heterologous peptides include, for example, a nuclear localization signal (NLS) such as the SV40 NLS for targeting to the nucleus (e.g., see Lange et al., J. Biol. Chem. 282:5101-5105, 2007). Such subcellular localization signals can be located at the N-terminus, the C-terminus, or anywhere within the Cas protein. An NLS can include a stretch of basic amino acids and can be a monopartite sequence or a bipartite sequence.

In particular embodiments, a Cas protein can also include a heterologous polypeptide for ease of tracking or purification, such as a fluorescent protein, a purification tag, or an epitope tag. Examples of tags include green fluorescent protein (GFP), glutathione-S-transferase (GST), myc, Flag, hemagglutinin (HA), Nus, Softag 1, Softag 3, Strep, polyhistidine, biotin carboxyl carrier protein (BCCP), maltose binding protein (MBP), and calmodulin.

Additional information regarding CRISPR-Cas systems and components thereof are described in U.S. Pat. Nos. 8,697,359, 8,771,945, 8,795,965, 8,865,406, 8,871,445, 8,889,356, 8,889,418, 8,895,308, 8,906,616, 8,932,814, 8,945,839, 8,993,233, 8,999,641, and applications related thereto; and International Patent Publications WO2014/018423, WO2014/093595, WO2014/093622, WO2014/093635, WO2014/093655, WO2014/093661, WO2014/093694, WO2014/093701, WO2014/093709, WO2014/093712, WO2014/093718, WO2014/145599, WO2014/204723, WO2014/204724, WO2014/204725, WO2014/204726, WO2014/204727, WO2014/204728, WO2014/204729, WO2015/065964, WO2015/089351, WO2015/089354, WO2015/089364, WO2015/089419, WO2015/089427, WO2015/089462, WO2015/089465, WO2015/089473, WO2015/089486, WO2016/205711, WO2017/106657, WO2017/127807, and applications related thereto.

Teachings of the disclosure in relation to CRISPR can be applied to other gene editing systems that similarly utilize nucleases.

Embodiments utilize zinc finger nucleases (ZFNs) as gene editing agents. ZFNs are a class of site-specific nucleases engineered to bind and cleave DNA at specific positions. ZFNs are used to introduce double strand breaks (DSBs) at a specific site in a DNA sequence which enables the ZFNs to target unique sequences within a genome in a variety of different cells. Moreover, subsequent to double-stranded breakage, homology-directed repair (HDR) or non-homologous end joining (NHEJ) takes place to repair the DSB, thus enabling genome editing.

ZFNs are synthesized by fusing a zinc finger DNA-binding domain to a DNA cleavage domain. The DNA-binding domain includes three to six zinc finger proteins which are transcription factors. The DNA cleavage domain includes the catalytic domain of, for example, FokI endonuclease. The FokI domain functions as a dimer requiring two constructs with unique DNA binding domains for sites on the target sequence. The FokI cleavage domain cleaves within a five or six base pair spacer sequence separating the two inverted half-sites.

Additional information regarding ZFNs can be found, for instance, in Kim et al., Proc Natl Acad Sci USA 93:1156-1160, 1996; Wolfe et al., Ann Rev Biophysics Biomol Struct. 29:183-212, 2000; Bibikova et al., Science 300:764, 2003; Bibikova et al., Genetics 161:1169-1175, 2002; Miller et al., EMBO J. 4:1609-1614, 1985; and Miller et al. Nature Biotech 25:778-785, 2007.

Embodiments can use transcription activator like effector nucleases (TALENs) as gene editing agents. TALENs refer to fusion proteins including a transcription activator-like effector (TALE) DNA binding protein and a DNA cleavage domain. TALENs are used to edit genes and genomes by inducing DSBs in the DNA, which induce repair mechanisms in cells. Generally, two TALENs must bind and flank each side of the target DNA site for the DNA cleavage domain to dimerize and induce a DSB. The DSB is repaired in the cell by NHEJ or HDR if an exogenous double-stranded donor DNA fragment is present.

Additional gene editing agents include transcription activator-like effector nucleases (TALENs). TALENs refer to fusion proteins including a transcription activator-like effector (TALE) DNA binding protein and a DNA cleavage domain. TALENs are used to edit genes and genomes by inducing double strand breaks (DSBs) in the DNA, which induce repair mechanisms in cells. Generally, two TALENs must bind and flank each side of the target DNA site for the DNA cleavage domain to dimerize and induce a DSB. The DSB is repaired in the cell by non-homologous end-joining (NHEJ) or by homologous recombination (HR) with an exogenous double-stranded donor DNA fragment.

As indicated, TALENs have been engineered to bind a target sequence of, for example, an endogenous genome, and cut DNA at the location of the target sequence. The TALEs of TALENs are DNA binding proteins secreted by Xanthomonas bacteria. The DNA binding domain of TALEs include a highly conserved 33 or 34 amino acid repeat, with divergent residues at the 12th and 13th positions of each repeat. These two positions, referred to as the Repeat Variable Diresidue (RVD), show a strong correlation with specific nucleotide recognition. Accordingly, targeting specificity can be improved by changing the amino acids in the RVD and incorporating nonconventional RVD amino acids.

Examples of DNA cleavage domains that can be used in TALEN fusions are wild-type and variant FokI endonucleases. The FokI domain functions as a dimer requiring two constructs with unique DNA binding domains for sites on the target sequence. The FokI cleavage domain cleaves within a five or six base pair spacer sequence separating the two inverted half-sites.

Particular embodiments utilize MegaTALs as gene editing agents. MegaTALs have a single chain rare-cleaving nuclease structure in which a TALE is fused with the DNA cleavage domain of a meganuclease. Meganucleases, also known as homing endonucleases, are single peptide chains that have both DNA recognition and nuclease function in the same domain. In contrast to the TALEN, the megaTAL only requires the delivery of a single peptide chain for functional activity.

### Post-Transcriptional Inhibition of AR Expression

In another embodiment AR expression and/or activity is reduced through expression of a polynucleotide that encodes an AR-targeted hairpin RNA (hpRNA) that triggers RNA inhibition, which results in degradation of transcribed mRNA. In the cell, hpRNA molecules are modified through a ubiquitous enzymatic process to generate siRNA molecules. This enzymatic process may utilize an RNase Ill enzyme, such as DICER in eukaryotes, either in vitro or in vivo. See Elbashir et al., Nature 411:494-8, 2001; and Hamilton and Baulcombe, Science 286(5441):950-2, 1999. DICER or functionally-equivalent RNase Ill enzymes cleave larger dsRNA strands and/or hpRNA molecules into smaller oligonucleotides, siRNAs and miRNAs. The siRNA molecules produced by these enzymes have 2 to 3 nucleotide 3′ overhangs, and 5′ phosphate and 3′ hydroxyl termini. The siRNA molecules generated by RNase Ill enzymes are unwound and separated into single-stranded RNA in the cell. The siRNA molecules then specifically hybridize with mRNAs transcribed from the target gene, and both RNA molecules are subsequently degraded by an inherent cellular RNA-degrading mechanism. This process results in the effective degradation or removal of the mRNA encoded by the target gene in the target organism. The outcome is the post-transcriptional silencing of the targeted gene. In embodiments herein, siRNA molecules produced by endogenous RNase Ill enzymes from the hpRNAs of the disclosure efficiently mediate the inhibition of AR in a target immune cell or population of immune cells, in vivo or ex vivo. Additional information can be found for instance in: Chiu & Rana (Mol. Cell, 10(3):549-561, 2002), Robb et al. (Nature Struct & Mol Biol., 12:133-137, 2005), Shtam et al. (Cell Commun. Signal. 11(88), 2013).

An hpRNA molecule herein may be transcribed from a polynucleotide containing an antisense nucleotide sequence that encodes any of the foregoing antisense polyribonucleotides; a sense nucleotide sequence that is substantially identical or identical to the antisense polyribonucleotide; and an intervening polyribonucleotide positioned between the sense and the antisense polyribonucleotides, such that the sense and antisense polyribonucleotides in the transcript of the polynucleotide hybridize to form all or part of a “stem” structure in the hpRNA molecule, and the polyribonucleotide transcribed from the intervening sequence forms a “loop.” For ease of explanation, these polyribonucleotides may be listed in the order in which they appear in the 5′ to 3′ direction in the hpRNA: the first polyribonucleotide, a second polyribonucleotide (the intervening, spacer polyribonucleotide), and a third polyribonucleotide (substantially identical or identical to the complement or reverse complement of the first polyribonucleotide). In some embodiments, the hpRNA molecule includes a plurality of such sense and corresponding antisense polyribonucleotides present in the stem of the hpRNA, which may be, for example, separated by intervening sequences in each strand of the stem. In some embodiments, the sense and corresponding antisense polyribonucleotides have different lengths.

The intervening, spacer polyribonucleotide may include any suitable sequence that facilitates secondary structure formation between the polyribonucleotides of the stem structure. In some examples, the spacer is part of a sense or antisense polyribonucleotide in the hpRNA. In further examples, the spacer is an intron. In some embodiments, however, the hpRNA does not include a spacer. Many suitable spacers are known and widely-used in the art to engineer hpRNAs that are processed into siRNAs in a cell, and any of the spacers may be used in embodiments herein, according to the discretion of the practitioner.

### Pharmacological Inhibition of AR Activity or Function

Also provided are embodiments in which the AR reduction is provided through pharmacological intervention, for instance using a chemical inhibitor of androgen synthesis and/or the androgen receptor. AR inhibitors include AR antagonists or AR degraders. AR degraders result in the physical breakdown of the AR receptor. AR antagonists reduce or block the activity of AR by inhibiting AR binding and/or otherwise functionally disrupting AR signaling. Generally speaking, an AR antagonist is a substance that keeps androgens from binding to the AR protein. These substances can include compounds that perturb the enzymatic activity of proteins involved in cholesterol steroidogenesis including 5-alpha reductase, CYP17a inhibitors, and HSDB1 inhibitors. Preventing production of the androgen hormone and/or its binding blocks the effects of these hormones in the cell or the body.

The art recognizes a number of AR inhibitors and antagonists, including but not limited to: apalutamide (an AR antagonist that is used in the treatment of prostate cancer), bicalutamide (an androgen receptor inhibitor that is used, for instance, to treat Stage D2 metastatic carcinoma of the prostate), clascoterone (an AR antagonist used, for instance, for the topical treatment of acne vulgaris), enzalutamide (an AR inhibitor that has been used to treat castration-resistant prostate cancer), flutamide (an antiandrogen that has been used, for instance, for locally confined stage B2-C and D-2 metastatic prostate carcinoma), and nilutamide (an antineoplastic hormone, currently used to treat prostate cancer). Additional information about compounds that can be used to inhibit activity of AR can be found, for instance, in: Song et al. (Cell Mol Life Sci 77(22):4663-4673, 2020), Tan et al. (Acta Pharma Sinica 36:3-23, 2015), Armstrong & Gao (AmJ Clin Exp Urol., 9(4):292-300, 2021), Mohler et al. (Int J Mol Sci. 22(4):2124, 2021), Ban et al. (Cancers 13(14):3488, 2021).

Examples of direct and/or indirect AR inhibitors (including compounds that reduce AR activity by inhibiting androgen synthesis which then reduces AR activity) include abiraterone (or CB-76 30; (3S,8R,9S, 1 OR, 13S, 14S)-10,13-dimethyl-17-(pyridin-3-yl) 2,3,4,7,8,9,10,11,12,13,14,15-dodecahydro-1H-cyclopenta [a]phenanthren-3-ol); apalutamide (ERLEADA, Janssen Biotech, Inc); ARN-509; ASC-J9; bevacizumab (Avastin; a monoclonal antibody that recognizes and blocks vascular endothelial growth factor A (VEGF-A)); bexlosteride (LY-191,704; (4aS,10bR)-8-chloro-4-methyl-1,2,4a,5,6,10b-hexahydrobenzo[f]quinolin-3-one); bicalutamide (N-[4-cyano-3-(trifluoromethyl)phenyl]-3-[(4-fluorophenyl)sulfonyl]-2-hydroxy-2-methylpropanamide); cabazitaxel (XRP-6258); cyproterone acetate (6-chloro-ip,2p-dihydro-17-hydroxy-3′H-cyclopropa[1,2]pregna-4,6-diene-3,20-dione); darolutamide (NUBEQA; also, Keto-darolutamide which is a metabolite of Darolutamid); docetaxel (Taxotere; 1,7β,10β-trihydroxy-9-oxo-5β,20-epoxytax-1 I-cnc-2a,4,13a-triyl 4-acetate 2-benzoate 13-{(2R,3S)-3-[(tert-butoxycarbonyl)amino]-2-hydroxy-3-phenylpropanoate}); dutasteride (Avodart; N-[2,5-Bis(trifluoromethyl)phenyl]-3-oxo-4-aza-5α-androst-1-ene-17β-carboxamide); enzalutamide (MDV3100; (4-(3-(4-cyano-3-(trifluoromethyl)phenyl)-5,5-dimethyl-4-oxo-2-thioxoimidazolidin-I-yl)-2-fluoro-N-methyl benzamide)); FCE 28260; finasteride (Proscar, Propecia; N-tert-Butyl-3-oxo-4-aza-5α-androst-1-ene-17β-carboxamide or N-(I,I-dimethylethyl)-3-oxo-(5a, 17P)-4-azaandrost-1-ene-17-carboxamide); flutamide (2-methyl-N-[4-nitro-3-(trifluoromethyl)phenyl]-propanamide); galeterone; hydroxyflutamide; izonsteride (LY-320,236; (4aR, 10bR)-8-[(4-ethyl-1,3-benzothiazol-2-yl)sulfanyl]-4,1 Ob-dimethyl-1,4,4a,5,6,10b-hexahydrobenzo[f]quinolin-3(2H)-one); MDX-010 (Ipilimumab, a fully human monoclonal antibody that binds to and blocks the activity of CTLA-4); nilutamide (5,5-dimethyl-3-[4-nitro-3-(trifluoromethyl)phenyl]imidazolidine-2,4-dione); ODM-201; OSU-HDAC42 ((S)-(-r)-N-hydroxy-4-(3-methyl-2-phenylbutyrylamino)-benzamide); SKF105,111; sunitumib (N-(2-diethylaminoethyl)-5-[(Z)-(5-fluoro-2-oxo-1H-indol-3-ylidene)methy 1]-2,4-dimethyl-1H-pyrrole-3-carboxamide); turosteride ((4aR,4bS,6aS,7S,9aS,9bS,11aR)-1,4a,6a-trimethyl-2-oxo-N-(propan-2-yl)-N-(propan-2ylcarbamoyl)hexadecahydro-IH-indeno[5,4-f]quinoline-7-carboxamide); vitaxin (a monoclonal antibody against the vascular integrin αvβ3); and ZD-4054 (N-(3-methoxy-5-methyl-2-pyrazinyl)-2-[4-(1,3,4-oxadiazol-2-yl)phenyl]-3-pyridinesulfonamide. For additional information regarding AR inhibitors, see Crona et al., Clin Pharmacol Ther. 2015; 98(6):582-589; Njar et al., Translational Cancer Research. 2017; 6, S7; Beretta, et al., Frontiers in chemistry vol. 7 369. 28 May 2019; Agnese, et al., J Cancer Metastasis Treat 2017; 3:328-61; U.S. Pat. No. 9,289,436B2; and AU2015266654A1.

Particular examples of AR degraders include proteolysis targeting chimera-like molecules (PROTAC) that recruit an E3 ligase for AR degradation. 2-63, disclosed herein, can act as one such AR degrader under GSH-deficient conditions described herein. 2-63 has a molecular weight of 694.2, well below the molecular weight of other PROTAC published thus far.

Examples of ITC include phenethyl-ITC, sulforaphane, erysolin, erucin, iberin, alyssin, berteroin, iberverin, cheirolin, 5-methylsulfinylpentyl-ITC, 6-methylsulfinylhexyl-ITC, 7-methylsulfinylheptyl-ITC, 8-methylsulfinyloctyl-ITC (hirsutin), 9-methylsulfinylnonyl-ITC, 10-methylsulfinyldecyl-ITC, phenylethyl-ITC, 4-(α.-L-rhamnopyranosyloxy)benzyl-ITC, 3-(α-L-rhamnopyranosyloxy)benzyl-ITC, 2-(α-L-rhamnopyranosyloxy)benzyl-ITC, and 4-(4′-O-acetyl-α-L-rhamnopyranosyloxy)benzyl-ITC.

### Compositions for Administration

The current disclosure provides compositions, including a drug and/or drug combination disclosed herein. In particular embodiments, the composition is a pharmaceutical composition including a drug and/or drug combination disclosed herein and a pharmaceutically acceptable carrier. For example, a composition disclosed herein could include an inhibitor of AR activity, a compound useful in androgen deprivation therapy, a compound used in blockade therapy (such as PD-L1/PD-1 blockade), and/or an epigenetic modifier compound. Other exemplary compounds are described herein.

Each drug can be formulated into its own composition for administration, or the drug can be formulated with an additional active ingredient for administration as a composition. The drugs described herein, and the additional active ingredient can be formulated for use in a combination therapy.

For injection, compositions can be formulated as aqueous solutions, such as in buffers, including Hanks' solution, Ringer's solution, or physiological saline. The aqueous solutions can contain formulatory agents such as suspending, stabilizing, and/or dispersing agents. Examples of suitable aqueous and non-aqueous carriers, which may be employed in the injectable formulations include water, ethanol, polyols (such as glycerol, propylene glycol, polyethylene glycol, and the like), and suitable mixtures thereof, vegetable oils, such as olive oil, and injectable organic esters, such as ethyloleate. Proper fluidity can be maintained, for example, by the use of coating materials, such as lecithin, by the maintenance of selected particle size in the case of dispersions, and by the use of surfactants.

Injectable formulations may also contain adjuvants such as preservatives, wetting agents, emulsifying agents, and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like in the compositions.

Alternatively, the composition can be in lyophilized form and/or provided in powder form for constitution with a suitable vehicle, e.g., sterile pyrogen-free water, before use. Lyophilized compositions can include less than 5% water content, less than 4.0% water content, or less than 3.5% water content.

In particular embodiments, the composition can be in a unit dosage form, such as in a suitable diluent in sterile, hermetically sealed ampoules or sterile syringes.

In particular embodiments, in order to prolong the effect of a composition, it is desirable to slow the absorption of the active ingredient(s) following injection. Compositions can be formulated as sustained-release systems utilizing semipermeable matrices of solid polymers containing at least one administration form. Various sustained-release materials have been established and are well known by those of ordinary skill in the art. Sustained-release systems may, depending on their chemical nature, release active ingredients following administration for a few weeks up to over 100 days.

Compositions can also be formulated for oral administration. For ingestion, compositions can take the form of tablets, pills, lozenges, sprays, liquids, and capsules formulated in conventional manners. Ingestible compositions can be prepared using conventional methods and materials known in the pharmaceutical art. For example, U.S. Pat. Nos. 5,215,754 and 4,374,082 relate to methods for preparing swallowable compositions. U.S. Pat. No. 6,495,177 relates to methods to prepare chewable supplements with the improved mouthfeel. U.S. Pat. No. 5,965,162, relates to compositions and methods for preparing comestible units which disintegrate quickly in the mouth.

For administration by inhalation (e.g., nasal or pulmonary), the compositions can be formulated as aerosol sprays for pressurized packs or a nebulizer, with the use of suitable propellants, e.g. dichlorodifluoromethane, trichlorofluoromethane, or dichlorotetra-fluoroethane.

Nanoparticle formulations for a variety of administration routes can also be used.

Any composition described herein can advantageously include any other pharmaceutically acceptable carriers, which include those that do not produce significant adverse, allergic, or other untoward reactions that outweigh the benefit of administration, whether for research, prophylactic, and/or therapeutic treatments. Exemplary pharmaceutically acceptable carriers and formulations are disclosed in Remington's Pharmaceutical Sciences, 18th Ed. Mack Printing Company, 1990.

Moreover, formulations can be prepared to meet sterility, pyrogenicity, general safety, and purity standards required by the U.S. FDA Office of Biological Standards and/or other relevant foreign regulatory agencies.

In particular embodiments, the compositions can include, for example, 25 μg/mL or mg-5 mg/mL or mg, 50 μg/mL or mg-5 mg/mL or mg, 100 μg/mL or mg-5 mg/mL or mg, 150 μg/mL or mg-5 mg/mL or mg, 200 μg/mL or mg-5 mg/mL or mg, 250 μg/mL or mg-5 mg/mL or mg, 300 μg/mL or mg-5 mg/mL or mg, 350 μg/mL or mg-5 mg/mL or mg, 400 μg/mL or mg-5 mg/mL or mg, 450 μg/mL or mg-5 mg/mL or mg, 500 μg/mL or mg-5 mg/mL or mg, 550 μg/mL or mg-5 mg/mL or mg, 600 μg/mL or mg-5 mg/mL or mg, 650 μg/mL or mg-5 mg/mL or mg, 700 μg/mL or mg-5 mg/mL or mg, 750 μg/mL or mg-5 mg/mL or mg, 800 μg/mL or mg-5 mg/mL or mg, 850 μg/mL or mg-5 mg/mL or mg, 900 μg/mL or mg-5 mg/mL or mg, 950 μg/mL or mg-5 mg/mL or mg, 1 mg/mL or mg-5 mg/mL or mg, 1.5 mg/mL or mg-5 mg/mL or mg of one or more of the active ingredients.

### Methods of Use

The current disclosure utilizes the compositions disclosed herein to treat a subject diagnosed with cancer, or another disease or condition that are responsive to immune checkpoint therapy but in which the subject has become refractory to the therapy. In particular embodiments, the cancer is prostate cancer, or sarcoma, or any immunotherapy refractory solid tumor. In particular embodiments, the cancer is a high risk hormone sensitive disease.

As used herein, subjects include humans, veterinary animals (dogs, cats, reptiles, birds, etc.), livestock (horses, cattle, goats, pigs, chickens, etc.), and research animals (monkeys, rats, mice, fish, etc.).

Treating subjects includes delivering therapeutically effective amounts of the compositions disclosed herein. Therapeutically effective amounts can include effective amounts and therapeutic amounts.

An “effective amount” is the amount of active agent(s) or composition(s) necessary to result in a desired physiological change in a subject in vivo or in vitro. Effective amounts are often administered for research purposes. Effective amounts disclosed herein can have an effect on cell growth, for example, as determined by an MTT assay and/or colony formation assay. Cell counting as a golden standard can be performed routinely to determine cell doubling times and growth rates. Cell viability can be determined by trypan blue exclusion and LDH release assays.

Therapeutic amounts of the compositions disclosed herein can have an anti-cancer effect, particularly on AR positive cancer, including prostate cancer. Cancer (medical term: malignant neoplasm) refers to a class of diseases in which a group of cells displays uncontrolled growth (division beyond the normal limits), invasion (intrusion on and destruction of adjacent tissues), and sometimes metastasis. “Metastasis” refers to the spread of cancer cells from their original site of proliferation to another part of the body. For solid tumors, the formation of metastasis is a very complex process. It depends on the detachment of malignant cells from the primary tumor, invasion of the extracellular matrix, penetration of the endothelial basement membranes to enter the body cavity and vessels, and then, after being transported by the blood or lymph, infiltration of target organs. Finally, the growth of a new tumor, i.e., a secondary tumor or metastatic tumor, at the target site depends on angiogenesis. Tumor metastasis often occurs even after removing the primary tumor because tumor cells or components may remain and develop metastatic potential.

A “tumor” is swelling or lesion formed by an abnormal growth of cells (called neoplastic cells or tumor cells). A “tumor cell” is an abnormal cell divided by a rapid, uncontrolled cellular proliferation and continues to divide after the stimuli that initiated the new division ceases. Tumors show partial or complete lack of structural organization and functional coordination with the normal tissue. Usually, they form a distinct mass of tissue, either benign, pre-malignant, or malignant.

As used herein, an anti-cancer effect refers to a biological effect, which can be manifested by a decrease in tumor volume, a decrease in the number of cancer cells, a decrease in the number of metastases, an increase in life expectancy, or a decrease of various physiological symptoms associated with the cancerous condition. An anti-cancer effect can also be manifested by a decrease in recurrence or an increase in the time before recurrence.

For administration, therapeutically effective amounts (also referred to herein as doses) can be initially estimated based on results from in vitro assays, animal model studies, and/or preclinical studies. Such information can be used to determine useful doses in subjects of interest more accurately. Particularly useful pre-clinical tests include a measure of cell growth, cell death, and/or cell viability.

The actual dose amount administered to a particular subject can be determined by a physician, veterinarian, or researcher taking into account parameters such as physical, physiological, and psychological factors including target, body weight, stage of cancer, the type of cancer, previous or concurrent therapeutic interventions, idiopathy of the subject, and route of administration.

Exemplary doses can include 0.05 mg/kg to 10.0 mg/kg of one or more drugs disclosed herein. The total daily dose can be 0.05 mg/kg to 30.0 mg/kg of one or more drugs disclosed herein to a subject one to three times a day, including administration of total daily doses of 0.05-3.0, 0.1-3.0, 0.5-3.0, 1.0-3.0, 1.5-3.0, 2.0-3.0, 2.5-3.0, and 0.5-3.0 mg/kg/day of administration forms of a drug using 60-minute oral, intravenous or other dosing. In one particular example, doses can be administered QD or BID to a subject with, e.g., total daily doses of 1.5 mg/kg, 3.0 mg/kg, or 4.0 mg/kg of a composition with up to 92-98% wt/v of the compounds disclosed herein.

Additional useful doses can often range from 0.1 to 5 μg/kg or from 0.5 to 1 μg/kg. In other examples, a dose can include 1 μg/kg, 5 μg/kg, 10 μg/kg, 20 μg/kg, 30 μg/kg, 40 μg/kg, 50 μg/kg, 60 μg/kg, 70 μg/kg, 80 μg/kg, 90 μg/kg, 100 μg/kg, 150 μg/kg, 200 μg/kg, 250 μg/kg, 350 μg/kg, 400 μg/kg, 450 μg/kg, 500 μg/kg, 600 μg/kg, 700 μg/kg, 800 μg/kg, 900 μg/kg, 1000 μg/kg, 0.1 to 5 mg/kg or from 0.5 to 1 mg/kg. In other examples, a dose can include 1 mg/kg, 5 mg/kg, 10 mg/kg, 15 mg/kg, 20 mg/kg, 25 mg/kg, 30 mg/kg, 35 mg/kg, 40 mg/kg, 45 mg/kg, 50 mg/kg, or more.

Therapeutically effective amounts can be achieved by administering single or multiple doses during the course of a treatment regimen (e.g., hourly, every 2 hours, every 3 hours, every 4 hours, every 6 hours, every 9 hours, every 12 hours, every 18 hours, daily, every other day, every 3 days, every 4 days, every 5 days, every 6 days, weekly, every 2 weeks, every 3 weeks, or monthly).

The compositions described herein can be administered simultaneously or sequentially within a selected time window, such as within 10 minutes, 1 hour, 3 hours, 10 hours, 15 hours, 24 hours, or 48 hours' time windows or when the complementary active ingredient is administered within a clinically-relevant therapeutic window.

Compositions can also be administered with anesthetics including ethanol, bupivacaine, chloroprocaine, levobupivacaine, lidocaine, mepivacaine, procaine, ropivacaine, tetracaine, desflurane, isoflurane, ketamine, propofol, sevoflurane, codeine, fentanyl, hydromorphone, marcaine, meperidine, methadone, morphine, oxycodone, remifentanil, sufentanil, butorphanol, nalbuphine, tramadol, benzocaine, dibucaine, ethyl chloride, xylocaine, and/or phenazopyridine.

In particular embodiments, the compositions disclosed herein can be used in conjunction with other cancer treatments. For example, the composition disclosed herein can be administered in combination with other active ingredients, for example, an AR inhibitor (e.g., Enz, darolutamide, proxalutamide, apalutamide, biulatamide) a gonadotropin-releasing hormone agonist or antagonist (e.g., Lupron, Zoladex (Goserelin), Degarelix, Ozarelix, ABT-620 (Elagolix), TAK-385 (Relugolix), EP-100 or KLH-2109); a phosphoinositide 3-kinase (PI3K) inhibitor, a TORC inhibitor, or a dual PI3K/TORC inhibitor (e.g., BEZ-235, BKM120, BGT226, BYL-719, GDC0068, GDC-0980, GDC0941, GDC0032, MK-2206, OSI-027, CC-223, AZD8055, SAR245408, SAR245409, PF04691502, WYE125132, GSK2126458, GSK-2636771, BAY806946, PF-05212384, SF1126, PX866, AMG319, ZSTK474, Cal101, PWT33597, LY-317615 (enzastaurin hydrochloride), CU-906, or CUDC-907); a CYP17 inhibitor in addition to Galeterone (e.g., abiraterone acetate (Zytiga), TAK-700 (orteronel), or VT-464); prednisone; an osteoprotective agent; a radiation therapy; a kinase inhibitor (e.g., MET, VEGFR, EGFR, MEK, SRC, AKT, RAF, FGFR, CDK4/6); Provenge, Prostvac, Ipilimumab, a PD-1 inhibitor; a taxane or tubulin inhibitor; an anti-STEAP-1 antibody; a heat shock protein 90 (HSP90) or heat shock protein 27 (HSP27) pathway modulator; and/or immunotherapy.

Also specifically contemplated are embodiments in which the compositions disclosed herein are used in conjunction with a checkpoint inhibitor treatment(s). Immune checkpoint inhibitor treatments are known to those of skill in the art; representative examples are also mentioned herein. By way of example, targets for immune checkpoint inhibition include 2B4 (natural killer cell receptor 2B4), A2AR (Adenosine A2A receptor), B7-H3 (B7 Homolog 3; aka CD276), B7-H4 (B7 Homolog 4; VTCN1), BTLA (B- and T-lymphocyte attenuator), CTLA-4 (cytotoxic T lymphocyte antigen-4), IDO (Indoleamine 2,3-dioxygenase), KIR (Killer-cell Immunoglobulin-like Receptor), LAIRI (leukocyte-associated immunoglobulin-like receptor 1), LAG3 (lymphocyte-activation protein 3), NOX2 (NADPH oxidase 2), PD-1 (Programmed Death 1 receptor), SIGLEC7 (Sialic acid-binding immunoglobulin-type lectin 7; aka CD328), TIM-3 (T cell immunoglobulin mucin-3), TIGIT (T cell immunoreceptor with Ig and ITIM domains), and VISTA (V-domain Ig suppressor of T cell activation)). Exemplary checkpoint inhibitors include, but are not limited to, ipilimumab (Yervoy™) (which targets CTLA4); nivolumab (Opdivo™) perbrolizumab (Keytruda™), and cemiplimab (Libatyo™) (which target PD-1); and atezolizumab (Tecentriq™), Avelumab (Barvencio™), and durvalumab (Imfinzi™) (which target PD-L1).

As described herein, embodiments involve sex-specific (for instance, male subject-specific) treatments to improve responsiveness of the subjects to certain treatments. For instance, given the recognized that there are sex differences in effectiveness of DC vaccines for cancer (see Storkus et al., J. Immunother Cancer, 9:e003675, 2021; doi:10.1136/jitc-2021-003675), reduction in intrinsic AR level and/or function in DC of male patients (as taught herein) can be used to increase the effectiveness of treatments (such as antitumor treatment) in those patients.

### Screening, Detection, Panels, and Arrays

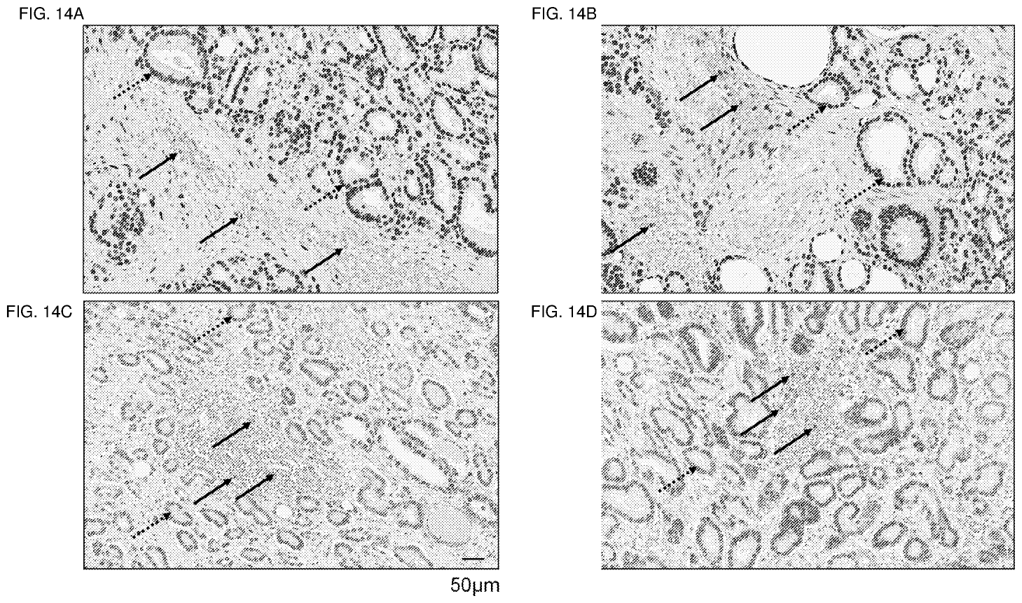

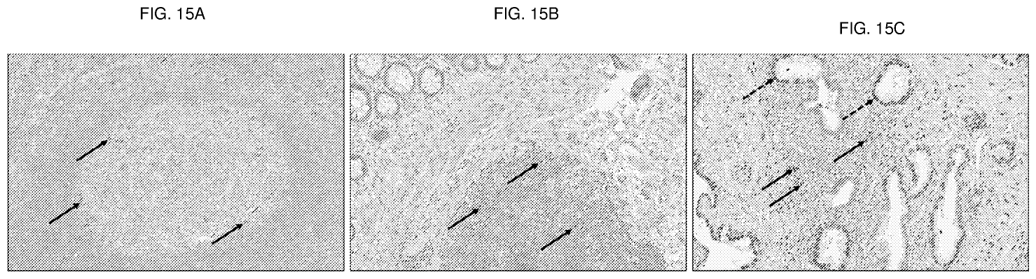

With the provision herein of the link between sex and suppression of immune cell function and disease conditions, and more particularly with the genes shown in Supplemental Table 4 in Appendix B of U.S. Provisional Application No. 63/319,712 (Column D) and disease conditions, there are enabled methods of using markers that detect and/or identify/characterize the androgen receptor in a sample from a subject. See, for instance, FIGS. 14A-14D and 15A-15C, which demonstrate AR protein staining in some tumor-infiltrating lymphocytes. Such methods include diagnosis, screening, and so forth, as described herein. These methods can be carried out using individual transcriptional or protein markers, or combinations of markers, including panels and arrays of multiple associated genes. Such panels may include inference of androgen receptor transcription factor activity based on the enrichment of the transcription factor's gene targets that are indicative of androgen receptor signaling. Supplemental Table 4 in Appendix B of U.S. Provisional Application No. 63/319,712 shows differentially expressed genes from CD8_R vs _NR. Genes with a positive value (in COL. D) are ‘up’ in AR_{low} T cells and thus associated with a positive response to checkpoint blockade. These markers (and subset thereof) identify reduction in androgen receptor expression, and therefore provide information on the activity of androgen receptor signaling (which provides insight regarding possible treatment resistance or sensitivity potential, for instance). Genes with a negative value in the same column are “down” in AR_{low} T cells, and thus associated with a negative response to checkpoint blockade. Panels and arrays that include fewer than all of the listed markers for any category (for instance, markers that show up regulation, or that show down regulation) are contemplated.

Androgen receptor activity wherein activity is determined for one or more immune cell or cell within the population of immune cells expressing AR, or AR activity is calculated based on master regulator (MR) analysis inferring differentially activated transcription factors in a defined gene signature (i.e. CD8_R vs. CD8_NR) based on the enrichment of each transcription factors gene targets. In addition, immune cell(s) associated with the tumor that are AR^{low} IFNγ^{hi} as a positive indicator of effective immunotherapy and AR^{hi}IFNγ^{low} as a biomarker of resistance to immunotherapy.

By way of example, Table 1 includes a list of differentially expressed genes that meet a minimum fold threshold ‘up’ (positive log FC(KO/WT); gene symbol in bold below) or ‘down’ (negative log FC(KO/WT)) in the herein-described AR KO T cell data. These genes (and the human equivalents in Table 1) are direct targets of AR activity, and can be included in an AR activity signature. AR deficient and AR WT CD8 T cells underwent RNA sequencing after activation. Differentially expressed genes were determined by natural log fold change in expression (determined by read counts) in the AR KO T cells over the WT CD8 T cells.

TABLE 1
genes UP (logFC(KO/WT) > 0) or DOWN (logFC(KO/WT) < 0) in AR KO T cells

| ensembl | | | | | | |
|---|---|---|---|---|---|---|
| ENSMUSG | logFC(KO/WT) | logCPM | PValue | gene_symbol | KO_cpm | WT_cpm |
| 00000060586 | 1.12180797 | 0.93400835 | 0.00890831 | H2-Eb1 | 2.60267266 | 1.19567456 |
| 00000036594 | 1.06516905 | 1.11006331 | 0.01283854 | H2-Aa | 2.90597624 | 1.38852529 |
| 00000020383 | 1.05563669 | 1.79802738 | 0.01255225 | Il13 | 4.68071208 | 2.25153233 |
| 00000017493 | 1.03608885 | 5.37412274 | 0.01254904 | Igfbp4 | 55.7422805 | 27.1823111 |
| 00000020641 | 1.02845578 | 0.91082536 | 0.01647686 | Rsad2 | 2.50840262 | 1.22942343 |
| 00000058603 | 0.99492093 | 1.95019258 | 0.01800122 | Rpl28-ps1 | 5.13156876 | 2.57455731 |
| 00000044285 | 0.99454489 | 0.62357832 | 0.0209385 | Gm1821 | 2.03705246 | 1.02210889 |
| 00000034947 | 0.9703318 | 0.83806691 | 0.02335275 | Tmem106a | 2.35265213 | 1.20049582 |
| 00000105388 | 0.95901604 | 1.68985867 | 0.02322292 | Rpl36a-ps2 | 4.24625019 | 2.18403457 |
| 00000108307 | 0.95876478 | 0.95713552 | 0.02478131 | Ceacam-ps1 | 2.54938959 | 1.311385 |
| 00000047293 | 0.9456615 | 1.41332806 | 0.02564855 | Gpr15 | 3.49208992 | 1.81279691 |
| 00000085912 | 0.94341408 | 0.62325017 | 0.02915997 | Trp53cor1 | 2.01246027 | 1.04621524 |
| 00000095687 | 0.9340979 | 3.79672786 | 0.02457897 | Rnaset2a | 18.2310047 | 9.5412901 |
| 00000070343 | 0.92482218 | 0.9453871 | 0.03005221 | Gm10288 | 2.50840262 | 1.32102753 |
| 00000113061 | 0.8794884 | 1.54959873 | 0.03736754 | AL607105.1 | 3.77899872 | 2.05386032 |
| 00000030208 | 0.8686029 | 2.07061381 | 0.03842579 | Emp1 | 5.41437886 | 2.96508005 |
| 00000076905 | −1.1735638 | −0.4201751 | 0.0090638 | Traj24 | 0.45495538 | 1.02693016 |
| 00000079625 | −1.1756059 | 0.14874537 | 0.00764923 | Tm4sf19 | 0.67628502 | 1.52834207 |
| 00000065219 | −1.1852581 | −0.2770586 | 0.00786126 | Snord32a | 0.50004105 | 1.13781934 |
| 00000111424 | −1.19635 | 0.54286789 | 0.00587799 | AC159308.5 | 0.88121988 | 2.02011145 |
| 00000086324 | −1.1987242 | 3.58094034 | 0.00412688 | Gm15564 | 7.25879255 | 16.6623035 |
| 00000103779 | −1.2038055 | 0.85924608 | 0.00519156 | Gm36931 | 1.09435212 | 2.52152336 |
| 00000110301 | −1.2039579 | 0.01241706 | 0.00643095 | Gm45599 | 0.60660717 | 1.39816783 |
| 00000082922 | −1.2048102 | 0.26956226 | 0.0059711 | Gm6939 | 0.72546939 | 1.67298012 |
| 00000097680 | −1.2132998 | −0.0203844 | 0.00587826 | Gm26642 | 0.59021238 | 1.36924022 |
| 00000019987 | −1.2270531 | −0.3203933 | 0.00652303 | Arg1 | 0.47544886 | 1.11371299 |
| 00000029379 | −1.2275717 | −0.2131261 | 0.00595883 | Cxcl3 | 0.51233714 | 1.20049582 |
| 00000096393 | −1.2291548 | 3.15529961 | 0.00334518 | Gm590 | 5.32420753 | 12.4822638 |
| 00000001227 | −1.2300349 | 0.44796648 | 0.00469122 | Sema6b | 0.81154203 | 1.90440101 |
| 00000054672 | −1.2301788 | 0.4979486 | 0.00478111 | 5830411N06Rik | 0.8402329 | 1.97189876 |
| 00000103649 | −1.2387489 | −0.0951864 | 0.00517746 | Gm37768 | 0.55332411 | 1.30656373 |
| 00000076918 | −1.2416153 | 0.31923743 | 0.00467148 | Traj11 | 0.73776548 | 1.74529915 |
| 00000102982 | −1.2517895 | 0.13023943 | 0.00443367 | Gm38319 | 0.64349544 | 1.53316334 |
| 00000113136 | −1.3038085 | 0.0024544 | 0.00339555 | AC133083.2 | 0.57381759 | 1.4174529 |
| 00000076907 | −1.3205867 | −0.4111986 | 0.00370751 | Traj22 | 0.4262645 | 1.06550031 |
| 00000110498 | −1.3308869 | −0.3632665 | 0.00308393 | Gm45803 | 0.43856059 | 1.10407046 |
| 00000091306 | −1.3466304 | −0.3125687 | 0.00273435 | Gm17606 | 0.45085668 | 1.14746187 |
| 00000035298 | −1.470016 | −0.3892294 | 0.00120606 | Klhl35 | 0.40167232 | 1.11371299 |
| 00000097300 | −1.5177668 | −0.524617 | 0.00096097 | Gm26835 | 0.35658665 | 1.02210889 |
| 00000110928 | −1.5575582 | 1.39200794 | 0.00029038 | AC132474.1 | 1.32797786 | 3.91004865 |
| 00000049985 | −1.6151546 | −0.5028701 | 0.0004314 | Ankrd55 | 0.34429056 | 1.05585777 |
| 00000031779 | −1.7774177 | 0.13074262 | 7.18E−05 | Ccl22 | 0.49184365 | 1.68744393 |
| 00000102672 | −1.8534954 | −0.4094566 | 5.46E−05 | Gm37105 | 0.32379707 | 1.17156821 |
| 00000016529 | −1.9794041 | −0.4839569 | 2.13E−05 | Il10 | 0.2869088 | 1.13299807 |

Key:
logFC (KO/WT): natural log fold change in expression (determined by read counts) in the AR KO T cells over the WT CD8 T cells; logCPM: log counts per million, a measure of expression; PValue: indication of statistical significance; KO_cpm: read counts in the AR KO T cells; WT_cpm: read counts in the WT CD8 T cells.

TABLE 2

| ensembl | | Human Gene |
|---|---|---|
| ENSMUSG | gene_symbol | Equivalent |
| 00000060586 | H2-Eb1 | HLA-A |
| 00000036594 | H2-Aa | HLA-B |
| 00000020383 | Il13 | HLA-C |
| 00000017493 | Igfbp4 | HLA-DRA |
| 00000020641 | Rsad2 | HLA-DRB |
| 00000058603 | Rpl28-ps1 | HLA-DQ |
| 00000044285 | Gm1821 | B2M |
| 00000034947 | Tmem106a | TAP1 |
| 00000105388 | Rpl36a-ps2 | TAP2 |
| 00000108307 | Ceacam-ps1 | TAPBP |
| 00000047293 | Gpr15 | IGFBP4 |
| 00000085912 | Trp53cor1 | GPR15 |
| 00000095687 | Rnaset2a | RNASET2A |
| 00000070343 | Gm10288 | TRP53COR1 |
| 00000019987 | Arg1 | ARG1d |
| 00000029379 | Cxcl3 | CXCL3 |
| 00000096393 | Gm590 | SEMA6B |
| 00000001227 | Sema6b | GM45803 |
| 00000031779 | Ccl22 | CCL22 |
| 00000016529 | Il10 | IL10 |

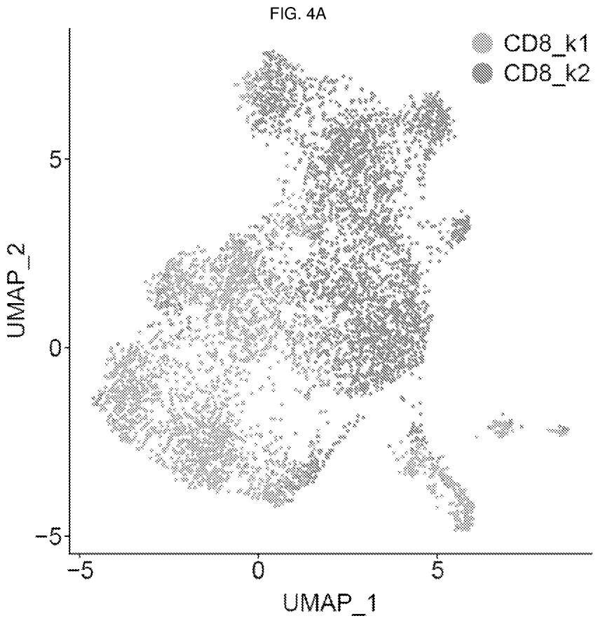

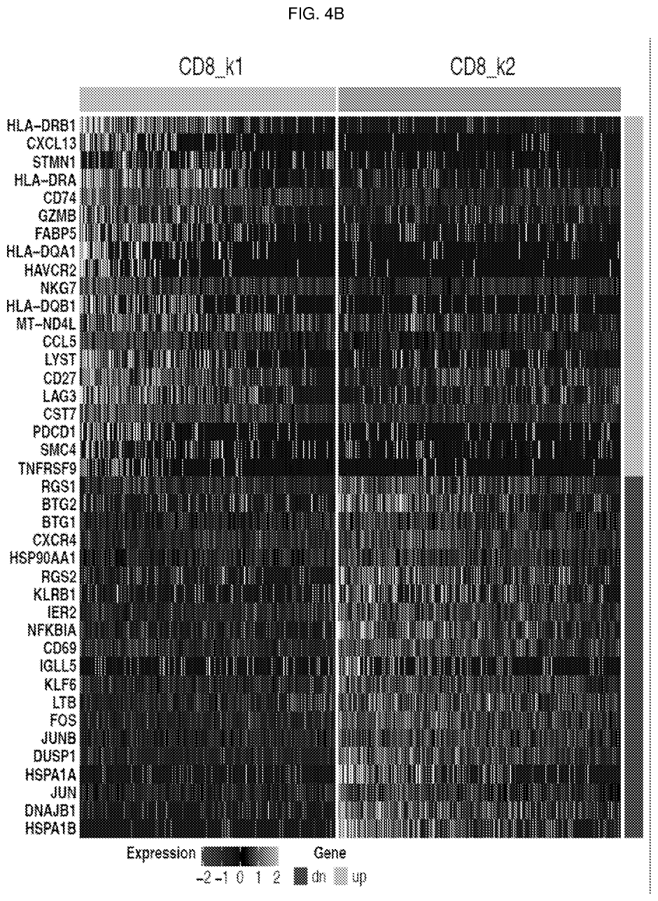

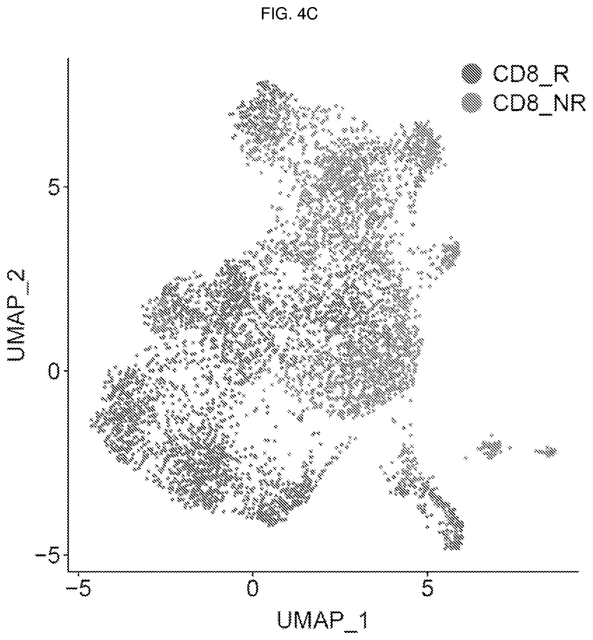

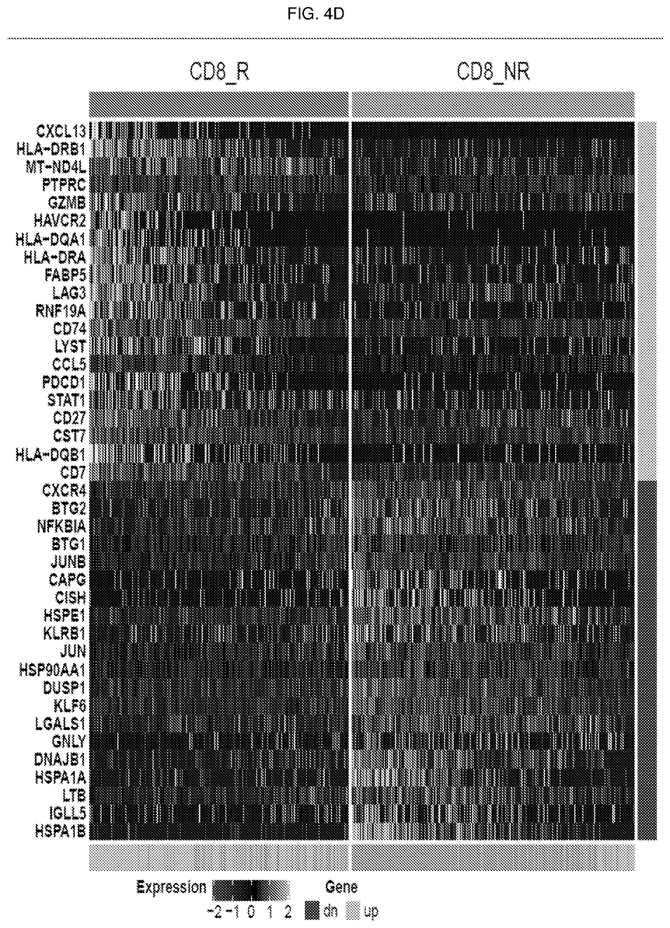

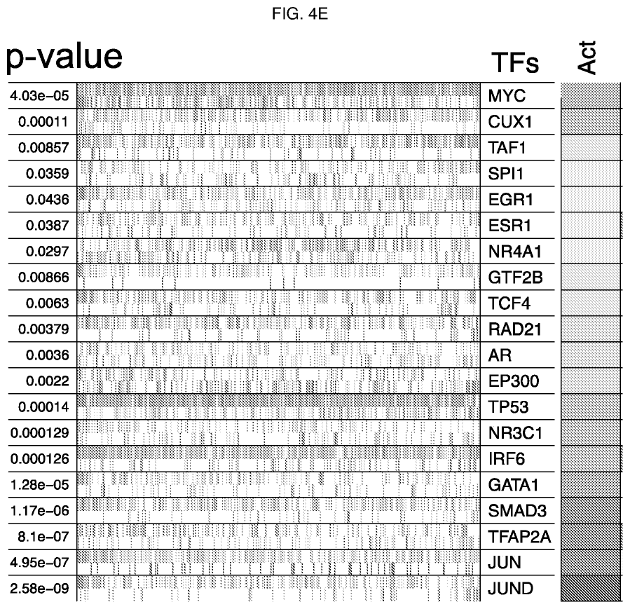

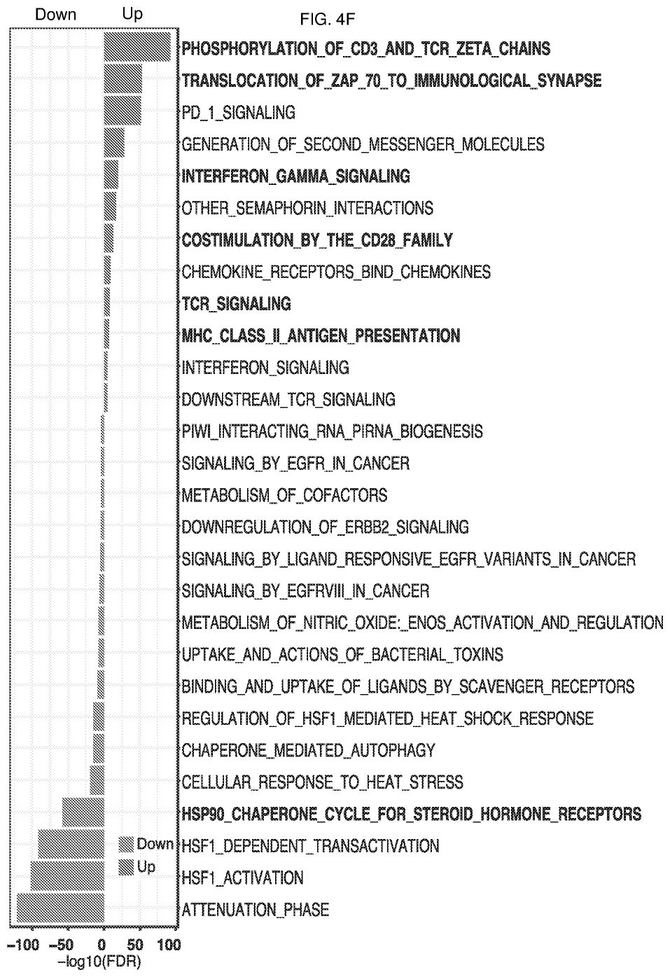

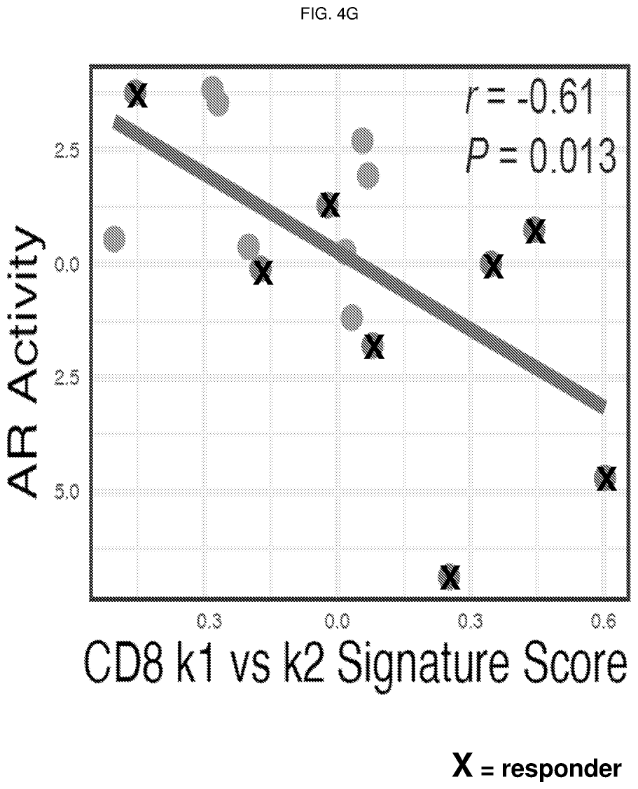

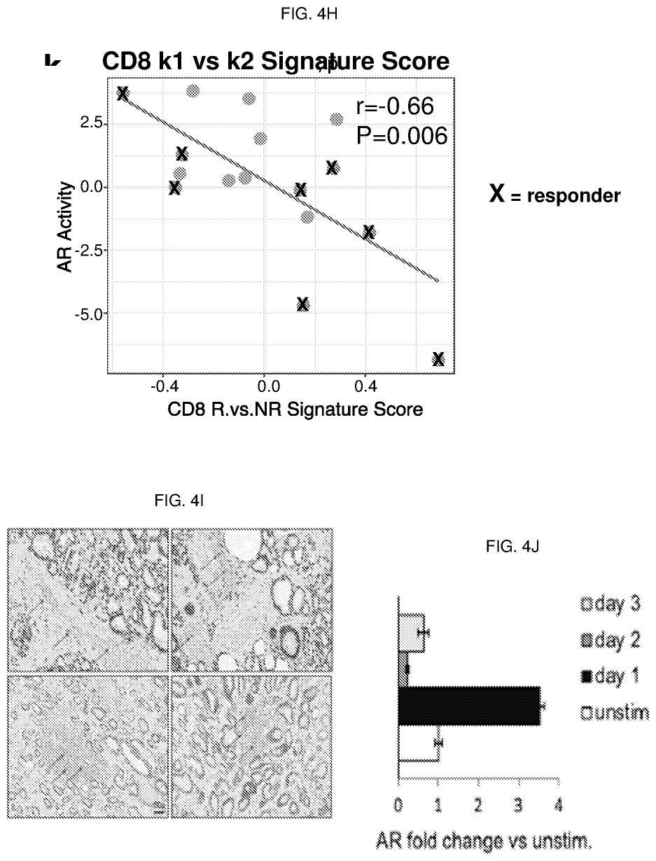

AR gene signatures include clusters of genes associated with 1) regulation of antigen presentation (major histocompatibility associated genes), as observed in differential expression of these genes (as listed in the following three Tables (previously provided in Appendix C): Table 3—Differentially expressed genes of CD8_k1 vs. CD8_k2, as defined using Seurat FindMarkers function (from FIGS. 4A-4B); Table 4—Differentially expressed genes of CD8_R vs. CD8_NR, as defined using Seurat FindMarkers function (form FIGS. 4C, 4D); and Table 5—up and down regulation of transcription factors (from FIG. 4E)); and 2) AR regulation of the expression of chemokine, chemokine receptors, integrins, and adhesion proteins involved in the migration of leukocytes such as Cxc13, Ccl22, Cxcr4; 3) AR regulation of inflammation such as IL13, IL10, IL6, Arg1. Subsets of genes would include increase in antigen presentation genes not limited to H2-eb1, H2-Aa and decrease in cytokines IL10, Arg1. Alternatively, increase in antigen presentation (H2-Eb1, H2-Aa) and decrease in chemokines (such as Ccl22, Cxc13).

TABLE 3
Differentially expressed genes of CD8_k1 vs. CD8_k2, as defined
using Seurat FindMarkers function, from FIGS. 4A-4B:

| gene | p_val | avg_logFC | pct. 1 | pct. 2 | p_val_adj |
|---|---|---|---|---|---|
| JUN | 0 | −1.3475773 | 0.52 | 0.91 | 0 |
| JUNB | 3.56E−296 | −1.2201849 | 0.467 | 0.833 | 4.55E−292 |
| HSPA1B | 9.30E−256 | −1.7557007 | 0.05 | 0.446 | 1.19E−251 |
| DUSP1 | 1.96E−242 | −1.2294019 | 0.26 | 0.673 | 2.50E−238 |
| BTG1 | 1.05E−237 | −0.7882934 | 0.721 | 0.923 | 1.35E−233 |
| DNAJB1 | 7.14E−229 | −1.3482502 | 0.145 | 0.54 | 9.14E−225 |
| HSP90AA1 | 1.49E−223 | −0.8269962 | 0.67 | 0.895 | 1.91E−219 |
| KLF6 | 4.09E−208 | −1.0529529 | 0.381 | 0.728 | 5.24E−204 |
| FOS | 4.61E−172 | −1.1584791 | 0.359 | 0.675 | 5.90E−168 |
| HLA-DRB1 | 4.72E−171 | 1.20470743 | 0.409 | 0.099 | 6.04E−167 |
| CD69 | 3.90E−160 | −0.9137683 | 0.372 | 0.686 | 4.99E−156 |
| EIF1 | 6.71E−147 | −0.4249581 | 0.93 | 0.981 | 8.59E−143 |
| IER2 | 5.43E−137 | −0.8689987 | 0.419 | 0.694 | 6.95E−133 |
| H3F3B | 7.17E−137 | −0.4872267 | 0.863 | 0.955 | 9.17E−133 |
| CCL5 | 1.09E−126 | 0.57274017 | 0.925 | 0.874 | 1.39E−122 |
| CXCR4 | 1.19E−126 | −0.8063525 | 0.42 | 0.697 | 1.53E−122 |
| UBC | 2.76E−124 | −0.5258114 | 0.688 | 0.871 | 3.53E−120 |
| HSPA1A | 2.63E−120 | −1.2403763 | 0.081 | 0.326 | 3.37E−116 |
| HLA-B | 2.64E−113 | 0.36538507 | 0.972 | 0.965 | 3.38E−109 |
| NFKBIA | 2.36E−110 | −0.8815058 | 0.213 | 0.48 | 3.02E−106 |
| RGS2 | 2.99E−100 | −0.8377235 | 0.164 | 0.417 | 3.83E−96 |
| CD74 | 4.59E−99 | 0.72716379 | 0.679 | 0.479 | 5.87E−95 |
| HLA-DRA | 3.93E−97 | 0.96697381 | 0.346 | 0.122 | 5.03E−93 |
| HSP90AB1 | 1.02E−96 | −0.5926468 | 0.475 | 0.695 | 1.31E−92 |
| RGS1 | 3.98E−94 | −0.7609314 | 0.379 | 0.631 | 5.10E−90 |
| ZFP36 | 6.54E−85 | −0.7455219 | 0.229 | 0.464 | 8.37E−81 |
| RPL12 | 5.92E−82 | 0.35348636 | 0.938 | 0.884 | 7.58E−78 |
| BTG2 | 4.42E−80 | −0.7736723 | 0.147 | 0.361 | 5.66E−76 |
| NKG7 | 2.10E−77 | 0.62016549 | 0.742 | 0.608 | 2.69E−73 |
| PPP1R15A | 7.46E−77 | −0.6746679 | 0.21 | 0.434 | 9.55E−73 |
| LTB | 1.51E−73 | −1.0680639 | 0.354 | 0.542 | 1.93E−69 |
| PTPRC | 2.94E−73 | 0.43259358 | 0.805 | 0.679 | 3.77E−69 |
| PMAIP1 | 7.98E−73 | −0.7383615 | 0.099 | 0.288 | 1.02E−68 |
| IFRD1 | 2.15E−70 | −0.7221468 | 0.081 | 0.257 | 2.75E−66 |
| VIM | 1.87E−69 | −0.4495185 | 0.594 | 0.756 | 2.39E−65 |
| CXCL13 | 5.12E−68 | 1.12765253 | 0.145 | 0.02 | 6.55E−64 |
| CAPG | 1.17E−66 | −0.6300927 | 0.061 | 0.225 | 1.50E−62 |
| LGALS1 | 2.74E−66 | −0.5772721 | 0.29 | 0.503 | 3.50E−62 |
| HSPE1 | 1.63E−65 | −0.6043142 | 0.318 | 0.515 | 2.09E−61 |
| GADD45B | 3.73E−65 | −0.6998302 | 0.152 | 0.345 | 4.78E−61 |
| HLA-DQA1 | 9.03E−65 | 0.64342513 | 0.163 | 0.031 | 1.16E−60 |
| ZFP36L2 | 1.44E−58 | −0.5766624 | 0.341 | 0.531 | 1.84E−54 |
| HAVCR2 | 4.24E−58 | 0.62889033 | 0.128 | 0.019 | 5.43E−54 |
| CST7 | 2.29E−57 | 0.50213475 | 0.615 | 0.433 | 2.93E−53 |
| SERTAD1 | 8.92E−56 | −0.6215844 | 0.074 | 0.223 | 1.14E−51 |
| CISH | 2.01E−55 | −0.585605 | 0.07 | 0.218 | 2.57E−51 |
| DNAJA1 | 1.10E−54 | −0.5595987 | 0.247 | 0.429 | 1.41E−50 |
| FABP5 | 1.68E−54 | 0.6762523 | 0.282 | 0.121 | 2.15E−50 |
| HLA-DQB1 | 1.24E−53 | 0.58696053 | 0.186 | 0.055 | 1.59E−49 |
| KLRB1 | 8.35E−50 | −0.8430996 | 0.156 | 0.317 | 1.07E−45 |
| LYST | 1.70E−49 | 0.55099304 | 0.244 | 0.097 | 2.18E−45 |
| DDX5 | 3.86E−48 | −0.3239093 | 0.72 | 0.816 | 4.94E−44 |
| MT-ND4L | 9.32E−48 | 0.58131517 | 0.371 | 0.211 | 1.19E−43 |
| DUSP2 | 5.47E−47 | −0.6311143 | 0.303 | 0.468 | 7.01E−43 |
| RPS9 | 7.96E−47 | 0.24822462 | 0.926 | 0.874 | 1.02E−42 |
| RGS16 | 2.64E−46 | −0.5740115 | 0.023 | 0.124 | 3.38E−42 |
| STMN1 | 2.65E−46 | 1.00999047 | 0.2 | 0.075 | 3.39E−42 |
| S100A10 | 1.65E−45 | −0.4098511 | 0.393 | 0.562 | 2.11E−41 |
| YPEL5 | 2.12E−45 | −0.5952962 | 0.153 | 0.307 | 2.71E−41 |
| FOSB | 2.55E−45 | −0.6011462 | 0.125 | 0.272 | 3.27E−41 |
| MYL12A | 5.47E−45 | −0.2897357 | 0.75 | 0.847 | 7.01E−41 |
| SLC2A3 | 2.80E−44 | −0.5479772 | 0.096 | 0.232 | 3.58E−40 |
| SRSF7 | 1.57E−43 | −0.4190095 | 0.551 | 0.695 | 2.01E−39 |
| CD27 | 1.11E−42 | 0.52716064 | 0.431 | 0.273 | 1.42E−38 |
| RPL37 | 3.62E−42 | 0.25909229 | 0.922 | 0.871 | 4.63E−38 |
| CD38 | 1.26E−40 | 0.4228852 | 0.113 | 0.025 | 1.61E−36 |
| LAG3 | 3.43E−39 | 0.51803871 | 0.323 | 0.179 | 4.39E−35 |
| HEXIM1 | 1.37E−38 | −0.4857317 | 0.07 | 0.186 | 1.76E−34 |
| DNAJB9 | 1.65E−38 | −0.4784247 | 0.08 | 0.2 | 2.12E−34 |
| CSRNP1 | 4.97E−38 | −0.4772981 | 0.033 | 0.128 | 6.36E−34 |
| HLA-DPA1 | 1.30E−37 | 0.42863351 | 0.449 | 0.295 | 1.66E−33 |
| COTL1 | 1.50E−37 | 0.41282341 | 0.591 | 0.468 | 1.92E−33 |
| ARL4A | 1.76E−37 | −0.5301212 | 0.062 | 0.171 | 2.25E−33 |
| ANXA1 | 5.16E−37 | −0.3798923 | 0.324 | 0.487 | 6.60E−33 |
| TRA2B | 5.61E−37 | −0.4609437 | 0.28 | 0.433 | 7.18E−33 |
| RPL14 | 4.98E−36 | 0.22748707 | 0.924 | 0.892 | 6.38E−32 |
| PLP2 | 6.83E−36 | −0.4095777 | 0.287 | 0.44 | 8.74E−32 |
| YME1L1 | 1.52E−35 | −0.4948542 | 0.119 | 0.245 | 1.94E−31 |
| GZMA | 2.97E−35 | 0.33757014 | 0.803 | 0.729 | 3.80E−31 |
| RP11-51J9.5 | 3.37E−35 | −0.4335245 | 0.041 | 0.136 | 4.31E−31 |
| HLA-DPB1 | 5.70E−35 | 0.40404176 | 0.509 | 0.364 | 7.30E−31 |
| TNFRSF9 | 1.05E−34 | 0.43399398 | 0.109 | 0.027 | 1.34E−30 |
| PDCD1 | 1.48E−34 | 0.48625284 | 0.158 | 0.059 | 1.90E−30 |
| CCNL1 | 4.65E−34 | −0.3858128 | 0.387 | 0.532 | 5.95E−30 |
| TSC22D3 | 6.63E−34 | −0.5169698 | 0.248 | 0.386 | 8.48E−30 |
| HLA-DMA | 2.99E−33 | 0.41133906 | 0.194 | 0.084 | 3.83E−29 |
| GZMB | 3.69E−33 | 0.72110889 | 0.311 | 0.183 | 4.72E−29 |
| NR4A2 | 4.98E−33 | −0.4723537 | 0.085 | 0.195 | 6.37E−29 |
| TNFSF13B | 9.11E−33 | −0.4379964 | 0.026 | 0.106 | 1.17E−28 |
| TSPYL2 | 1.41E−32 | −0.5023254 | 0.086 | 0.195 | 1.80E−28 |
| HLA-DRB5 | 2.56E−32 | 0.38495114 | 0.26 | 0.134 | 3.27E−28 |
| SRSF5 | 7.13E−32 | −0.3520651 | 0.501 | 0.63 | 9.12E−28 |
| PTGER2 | 1.10E−31 | −0.3534506 | 0.033 | 0.118 | 1.41E−27 |
| MT-ND2 | 7.67E−31 | −0.2382715 | 0.83 | 0.876 | 9.82E−27 |
| CLK1 | 2.14E−30 | −0.4087185 | 0.216 | 0.35 | 2.74E−26 |
| NACA | 4.46E−30 | 0.25089713 | 0.79 | 0.712 | 5.71E−26 |
| EIF4A2 | 8.31E−30 | −0.3107821 | 0.469 | 0.594 | 1.06E−25 |
| PERP | 1.30E−29 | −0.3908017 | 0.042 | 0.125 | 1.66E−25 |
| S100A6 | 1.94E−29 | −0.2482876 | 0.723 | 0.803 | 2.49E−25 |
| CITED2 | 3.02E−29 | −0.5067755 | 0.144 | 0.26 | 3.87E−25 |
| SBDS | 5.30E−29 | −0.4156401 | 0.128 | 0.243 | 6.79E−25 |
| ILF3-AS1 | 1.19E−28 | −0.4222473 | 0.1 | 0.207 | 1.52E−24 |
| PRMT10 | 1.22E−28 | −0.3991192 | 0.033 | 0.11 | 1.57E−24 |
| RPL7A | 2.19E−28 | 0.22829857 | 0.89 | 0.836 | 2.81E−24 |
| RARRES3 | 3.33E−28 | 0.31289059 | 0.595 | 0.472 | 4.26E−24 |
| EGR1 | 3.66E−28 | −0.4442409 | 0.029 | 0.102 | 4.68E−24 |
| CD7 | 3.97E−28 | 0.42085292 | 0.499 | 0.383 | 5.08E−24 |
| LINC00152 | 5.49E−28 | 0.35934445 | 0.344 | 0.213 | 7.03E−24 |
| ITM2A | 5.97E−28 | 0.42223154 | 0.504 | 0.395 | 7.65E−24 |
| PARK7 | 7.18E−28 | 0.38459291 | 0.483 | 0.368 | 9.18E−24 |
| RPS11 | 5.83E−27 | 0.22623735 | 0.832 | 0.767 | 7.46E−23 |
| DDX24 | 9.36E−27 | −0.3283702 | 0.396 | 0.526 | 1.20E−22 |
| TNFAIP3 | 1.10E−26 | −0.4190518 | 0.102 | 0.203 | 1.40E−22 |
| IDS | 1.78E−26 | −0.4146454 | 0.187 | 0.306 | 2.28E−22 |
| HMGB1 | 2.34E−26 | 0.35741507 | 0.739 | 0.679 | 2.99E−22 |
| C1orf63 | 2.80E−26 | −0.3467938 | 0.368 | 0.498 | 3.58E−22 |
| SMC4 | 3.30E−26 | 0.44578564 | 0.172 | 0.081 | 4.22E−22 |
| NUCB2 | 3.88E−26 | 0.39838103 | 0.231 | 0.126 | 4.97E−22 |
| GZMH | 3.94E−26 | 0.42738027 | 0.448 | 0.326 | 5.04E−22 |
| SAT1 | 4.80E−26 | −0.4585086 | 0.347 | 0.463 | 6.14E−22 |
| HSPA8 | 1.29E−25 | −0.2888825 | 0.583 | 0.674 | 1.66E−21 |
| XCL1 | 1.99E−25 | −0.3443998 | 0.104 | 0.206 | 2.55E−21 |
| SOCS1 | 2.43E−25 | −0.3653774 | 0.083 | 0.178 | 3.11E−21 |
| MGAT4A | 2.64E−25 | −0.334821 | 0.064 | 0.152 | 3.38E−21 |
| SIRPG | 7.39E−25 | 0.39588661 | 0.193 | 0.099 | 9.46E−21 |
| IFI44L | 1.68E−24 | −0.4521231 | 0.084 | 0.176 | 2.15E−20 |
| HSPA5 | 3.95E−24 | −0.3412407 | 0.255 | 0.375 | 5.06E−20 |
| CD9 | 5.05E−24 | −0.3122209 | 0.032 | 0.101 | 6.47E−20 |
| C1orf162 | 2.98E−23 | −0.2917527 | 0.039 | 0.11 | 3.81E−19 |
| RPL4 | 3.91E−23 | 0.25076397 | 0.725 | 0.635 | 5.00E−19 |
| RNF19A | 1.17E−22 | 0.38167169 | 0.227 | 0.129 | 1.49E−18 |
| AQP3 | 1.90E−22 | −0.3233541 | 0.035 | 0.1 | 2.44E−18 |
| RP11-489E7.4 | 2.31E−22 | −0.3770651 | 0.058 | 0.134 | 2.95E−18 |
| IFIT3 | 6.23E−22 | −0.587844 | 0.04 | 0.108 | 7.98E−18 |
| SNHG8 | 1.30E−21 | −0.3445748 | 0.145 | 0.246 | 1.66E−17 |
| MT-ND3 | 1.38E−21 | −0.2824502 | 0.617 | 0.695 | 1.77E−17 |
| PYHIN1 | 1.82E−21 | 0.33077865 | 0.346 | 0.232 | 2.33E−17 |
| TAF7 | 2.84E−21 | −0.3354568 | 0.289 | 0.399 | 3.64E−17 |
| MYADM | 3.10E−21 | −0.4180354 | 0.05 | 0.12 | 3.96E−17 |
| ITGB1 | 3.91E−21 | 0.40141096 | 0.222 | 0.132 | 5.00E−17 |
| TMPO | 6.08E−21 | 0.32011061 | 0.133 | 0.06 | 7.78E−17 |
| LUC7L3 | 1.26E−20 | 0.33524438 | 0.267 | 0.167 | 1.62E−16 |
| GBP2 | 2.94E−20 | 0.33368316 | 0.253 | 0.156 | 3.76E−16 |
| DDIT3 | 3.63E−20 | −0.3374637 | 0.044 | 0.109 | 4.65E−16 |
| NEU1 | 4.16E−20 | −0.3704068 | 0.067 | 0.141 | 5.33E−16 |
| DEK | 5.91E−20 | 0.34468304 | 0.323 | 0.226 | 7.57E−16 |
| TOX | 8.39E−20 | 0.30308486 | 0.121 | 0.053 | 1.07E−15 |
| LDHA | 8.81E−20 | −0.3151411 | 0.502 | 0.597 | 1.13E−15 |
| KLRD1 | 1.20E−19 | 0.43235927 | 0.208 | 0.122 | 1.54E−15 |
| ATP5G2 | 1.58E−19 | 0.26246281 | 0.579 | 0.492 | 2.03E−15 |
| IFI16 | 2.11E−19 | 0.27162564 | 0.511 | 0.41 | 2.70E−15 |
| H3F3A | 3.05E−19 | 0.22883521 | 0.77 | 0.692 | 3.90E−15 |
| BLOC1S1 | 3.05E−19 | 0.28672032 | 0.338 | 0.238 | 3.91E−15 |
| ZFAND5 | 3.73E−19 | −0.3411521 | 0.087 | 0.166 | 4.78E−15 |
| PTMS | 5.08E−19 | 0.33308029 | 0.159 | 0.083 | 6.50E−15 |
| TUBA1A | 5.68E−19 | −0.4117272 | 0.205 | 0.301 | 7.26E−15 |
| TLE4 | 8.03E−19 | −0.3346058 | 0.112 | 0.198 | 1.03E−14 |
| HNRNPDL | 1.31E−18 | −0.2576791 | 0.404 | 0.512 | 1.67E−14 |
| TIGIT | 1.33E−18 | 0.35079031 | 0.247 | 0.154 | 1.70E−14 |
| IVNS1ABP | 2.83E−18 | −0.2932048 | 0.044 | 0.105 | 3.62E−14 |
| CHMP1B | 3.05E−18 | −0.3460011 | 0.123 | 0.209 | 3.90E−14 |
| TIMP1 | 3.22E−18 | −0.279457 | 0.046 | 0.108 | 4.12E−14 |
| PTP4A1 | 3.60E−18 | −0.3283054 | 0.054 | 0.119 | 4.61E−14 |
| NCF1 | 4.89E−18 | −0.2831589 | 0.126 | 0.216 | 6.26E−14 |
| HIST1H1D | 6.38E−18 | 0.39528497 | 0.101 | 0.043 | 8.17E−14 |
| JUND | 8.15E−18 | −0.3133949 | 0.061 | 0.127 | 1.04E−13 |
| DDX17 | 9.73E−18 | 0.30499554 | 0.348 | 0.253 | 1.25E−13 |
| LBR | 1.01E−17 | 0.28874717 | 0.188 | 0.108 | 1.29E−13 |
| KIAA1551 | 1.01E−17 | 0.2938267 | 0.308 | 0.212 | 1.29E−13 |
| ID3 | 1.22E−17 | 0.34958847 | 0.136 | 0.068 | 1.56E−13 |
| LITAF | 2.74E−17 | 0.2993509 | 0.247 | 0.157 | 3.51E−13 |
| CKLF | 6.57E−17 | −0.2608501 | 0.463 | 0.551 | 8.41E−13 |
| HERPUD1 | 6.70E−17 | −0.3320195 | 0.169 | 0.256 | 8.58E−13 |
| FLT3LG | 6.95E−17 | −0.2855497 | 0.055 | 0.118 | 8.90E−13 |
| SOD1 | 7.18E−17 | 0.288173 | 0.509 | 0.422 | 9.18E−13 |
| CKS2 | 9.00E−17 | 0.38691015 | 0.123 | 0.061 | 1.15E−12 |
| GMFG | 9.07E−17 | 0.24302789 | 0.557 | 0.474 | 1.16E−12 |
| IGLL5 | 1.01E−16 | −1.0001342 | 0.121 | 0.201 | 1.29E−12 |
| ENO1 | 1.93E−16 | −0.3345904 | 0.429 | 0.516 | 2.47E−12 |
| SOCS3 | 2.19E−16 | −0.2702265 | 0.047 | 0.106 | 2.80E−12 |
| SRSF2 | 3.60E−16 | −0.288603 | 0.354 | 0.447 | 4.61E−12 |
| SLA | 5.43E−16 | 0.33121297 | 0.286 | 0.2 | 6.95E−12 |
| CD8A | 1.68E−15 | 0.27135756 | 0.5 | 0.412 | 2.15E−11 |
| IDH2 | 3.36E−15 | 0.30921242 | 0.277 | 0.195 | 4.30E−11 |
| SNAP47 | 4.66E−15 | 0.26950277 | 0.11 | 0.054 | 5.97E−11 |
| SYPL1 | 5.54E−15 | −0.2601215 | 0.071 | 0.135 | 7.09E−11 |
| TNF | 6.62E−15 | −0.2967655 | 0.091 | 0.161 | 8.47E−11 |
| RPS10 | 1.10E−14 | 0.2246124 | 0.575 | 0.485 | 1.41E−10 |
| GYPC | 1.47E−14 | 0.27387465 | 0.372 | 0.286 | 1.88E−10 |
| C9orf16 | 1.90E−14 | 0.26822163 | 0.357 | 0.271 | 2.44E−10 |
| NIN | 1.95E−14 | 0.25884439 | 0.127 | 0.067 | 2.49E−10 |
| PATL2 | 2.39E−14 | 0.28169503 | 0.1 | 0.048 | 3.06E−10 |
| IFI44 | 2.64E−14 | −0.2945109 | 0.086 | 0.151 | 3.38E−10 |
| CTSA | 2.80E−14 | −0.2967099 | 0.124 | 0.199 | 3.59E−10 |
| ARAP2 | 3.78E−14 | 0.26936713 | 0.203 | 0.128 | 4.84E−10 |
| ANP32B | 4.41E−14 | 0.30505534 | 0.296 | 0.218 | 5.64E−10 |
| PGK1 | 5.50E−14 | −0.286207 | 0.332 | 0.415 | 7.04E−10 |
| RGCC | 5.58E−14 | −0.2898191 | 0.216 | 0.3 | 7.14E−10 |
| SLC38A2 | 6.06E−14 | −0.2703565 | 0.095 | 0.162 | 7.75E−10 |
| LDLRAD4 | 6.51E−14 | −0.246525 | 0.093 | 0.161 | 8.33E−10 |
| PTTG1 | 7.06E−14 | 0.33831336 | 0.194 | 0.125 | 9.04E−10 |
| CYTIP | 8.47E−14 | −0.2747465 | 0.206 | 0.289 | 1.08E−09 |
| FUS | 9.48E−14 | −0.2241594 | 0.415 | 0.511 | 1.21E−09 |
| PABPC1 | 1.05E−13 | −0.2450988 | 0.459 | 0.536 | 1.34E−09 |
| NSD1 | 1.83E−13 | 0.24847359 | 0.189 | 0.12 | 2.34E−09 |
| PRF1 | 2.00E−13 | 0.28859591 | 0.275 | 0.196 | 2.56E−09 |
| HMGN2 | 2.05E−13 | 0.39081215 | 0.495 | 0.438 | 2.62E−09 |
| RHOC | 2.16E−13 | −0.2305217 | 0.137 | 0.213 | 2.76E−09 |
| LSM2 | 2.90E−13 | 0.24970833 | 0.221 | 0.148 | 3.72E−09 |
| ANXA2 | 7.22E−13 | −0.2522965 | 0.239 | 0.325 | 9.24E−09 |
| C1orf56 | 1.26E−12 | −0.280876 | 0.081 | 0.141 | 1.62E−08 |
| PIM1 | 1.99E−12 | −0.272549 | 0.151 | 0.223 | 2.55E−08 |
| NAP1L1 | 2.09E−12 | 0.22798075 | 0.443 | 0.363 | 2.67E−08 |
| RCAN3 | 2.37E−12 | −0.2447369 | 0.083 | 0.143 | 3.03E−08 |
| GSTP1 | 2.44E−12 | 0.25465908 | 0.333 | 0.254 | 3.12E−08 |
| PSMB8 | 2.85E−12 | 0.23757481 | 0.421 | 0.346 | 3.65E−08 |
| GBP5 | 3.07E−12 | 0.25034306 | 0.171 | 0.108 | 3.93E−08 |
| CXCR6 | 3.94E−12 | 0.31339358 | 0.197 | 0.133 | 5.04E−08 |
| TUBA4A | 4.43E−12 | −0.2809221 | 0.242 | 0.32 | 5.68E−08 |
| EIF1AX | 4.56E−12 | −0.2746324 | 0.118 | 0.182 | 5.84E−08 |
| ARF6 | 5.70E−12 | −0.2446164 | 0.229 | 0.307 | 7.29E−08 |
| NDUFC2 | 6.70E−12 | 0.24022163 | 0.306 | 0.232 | 8.57E−08 |
| RPS27L | 6.96E−12 | −0.2462038 | 0.347 | 0.427 | 8.90E−08 |
| EIF5 | 7.32E−12 | −0.2676596 | 0.227 | 0.303 | 9.37E−08 |
| ACP5 | 7.58E−12 | 0.23028431 | 0.107 | 0.058 | 9.70E−08 |
| CD44 | 8.15E−12 | −0.2236785 | 0.337 | 0.423 | 1.04E−07 |
| UCP2 | 8.68E−12 | 0.24854001 | 0.167 | 0.106 | 1.11E−07 |
| SNHG15 | 1.06E−11 | −0.2339009 | 0.064 | 0.116 | 1.36E−07 |
| MRPL51 | 1.10E−11 | 0.2256566 | 0.286 | 0.212 | 1.41E−07 |
| DNTTIP2 | 1.34E−11 | −0.2327174 | 0.11 | 0.173 | 1.71E−07 |
| LIMS1 | 1.62E−11 | 0.22347454 | 0.137 | 0.081 | 2.08E−07 |
| KLF10 | 3.06E−11 | −0.2379869 | 0.062 | 0.113 | 3.91E−07 |
| HNRNPA0 | 3.37E−11 | −0.240747 | 0.27 | 0.345 | 4.32E−07 |
| SLC38A1 | 3.42E−11 | 0.226534 | 0.176 | 0.115 | 4.38E−07 |
| CYCS | 5.31E−11 | −0.2467288 | 0.322 | 0.394 | 6.80E−07 |
| CLIC3 | 6.71E−11 | 0.28278048 | 0.13 | 0.078 | 8.59E−07 |
| TMEM123 | 6.98E−11 | −0.2434724 | 0.14 | 0.204 | 8.94E−07 |
| SLC4A7 | 7.37E−11 | 0.24046294 | 0.104 | 0.058 | 9.43E−07 |
| RP11-347P5.1 | 9.00E−11 | −0.2283361 | 0.076 | 0.129 | 1.15E−06 |
| BCAS2 | 9.26E−11 | −0.2541934 | 0.104 | 0.162 | 1.19E−06 |
| PRDX2 | 9.47E−11 | 0.32950619 | 0.23 | 0.166 | 1.21E−06 |
| CCDC59 | 1.08E−10 | −0.257179 | 0.122 | 0.183 | 1.39E−06 |
| CCL3 | 1.33E−10 | 0.4270258 | 0.213 | 0.151 | 1.70E−06 |
| RP11-94L15.2 | 1.68E−10 | 0.2532367 | 0.151 | 0.096 | 2.15E−06 |
| HSPH1 | 1.83E−10 | −0.2423922 | 0.073 | 0.122 | 2.35E−06 |
| PPM1K | 4.33E−10 | −0.2603687 | 0.103 | 0.159 | 5.54E−06 |
| TAGAP | 4.74E−10 | −0.2554322 | 0.064 | 0.111 | 6.07E−06 |
| MAGED2 | 5.03E−10 | −0.254248 | 0.109 | 0.165 | 6.44E−06 |
| CCL4 | 1.17E−09 | 0.36022091 | 0.554 | 0.483 | 1.50E−05 |
| LINC00861 | 2.07E−09 | 0.24527093 | 0.103 | 0.061 | 2.65E−05 |
| PAG1 | 2.31E−09 | 0.22750211 | 0.125 | 0.079 | 2.96E−05 |
| FAM3C | 3.14E−09 | 0.2380069 | 0.102 | 0.06 | 4.02E−05 |
| CDC42SE1 | 3.57E−09 | −0.2708733 | 0.131 | 0.187 | 4.57E−05 |
| SKIL | 3.91E−09 | −0.2394583 | 0.075 | 0.121 | 5.00E−05 |
| MX1 | 4.06E−09 | −0.3618172 | 0.132 | 0.186 | 5.20E−05 |
| CLDND1 | 5.43E−09 | −0.2448779 | 0.112 | 0.165 | 6.95E−05 |
| BATF | 2.75E−08 | 0.24279974 | 0.174 | 0.124 | 0.00035207 |
| MORF4L2 | 5.86E−08 | −0.2491911 | 0.124 | 0.173 | 0.00075037 |
| NT5C3A | 2.32E−07 | −0.2603252 | 0.09 | 0.133 | 0.00297178 |
| HIST1H1C | 2.44E−07 | 0.31323491 | 0.123 | 0.083 | 0.00311933 |
| H2AFZ | 3.93E−07 | 0.32939539 | 0.427 | 0.395 | 0.00502653 |
| XCL2 | 4.33E−07 | 0.40748426 | 0.193 | 0.147 | 0.00554544 |
| RILPL2 | 5.26E−07 | 0.23281223 | 0.132 | 0.092 | 0.00673487 |
| DUT | 2.01E−06 | 0.28039608 | 0.214 | 0.172 | 0.02572246 |

TABLE 4
Differentially expressed genes of CD8_R vs. CD8_NR, as defined
using Seurat FindMarkers function, from FIGS. 4C, 4D.

| gene | p_val | avg_logFC | pct. 1 | pct. 2 | p_val_adj |
|---|---|---|---|---|---|
| HLA-B | 3.86E−195 | 0.47168428 | 0.994 | 0.944 | 4.94E−191 |
| PTPRC | 7.91E−192 | 0.67908183 | 0.876 | 0.613 | 1.01E−187 |
| HSPA1B | 1.27E−186 | −1.4080821 | 0.083 | 0.418 | 1.63E−182 |
| JUN | 7.13E−159 | −0.6632896 | 0.596 | 0.843 | 9.13E−155 |
| HLA-DRB1 | 1.50E−154 | 1.13068374 | 0.401 | 0.104 | 1.92E−150 |
| HSP90AA1 | 6.15E−146 | −0.6759739 | 0.712 | 0.859 | 7.87E−142 |
| TMSB4X | 2.48E−117 | −0.2335978 | 0.999 | 0.993 | 3.17E−113 |
| KLF6 | 2.80E−117 | −0.7636576 | 0.434 | 0.682 | 3.59E−113 |
| CCL5 | 1.23E−115 | 0.5400652 | 0.946 | 0.854 | 1.57E−111 |
| BTG1 | 3.80E−115 | −0.5537378 | 0.775 | 0.875 | 4.86E−111 |
| CXCL13 | 1.55E−106 | 1.37557064 | 0.161 | 0.004 | 1.99E−102 |
| DNAJB1 | 2.12E−104 | −0.86847 | 0.217 | 0.476 | 2.71E−100 |
| MT-ND4L | 2.62E−104 | 0.80527946 | 0.417 | 0.167 | 3.36E−100 |
| DUSP1 | 2.89E−95 | −0.7010628 | 0.343 | 0.599 | 3.70E−91 |
| H3F3B | 6.03E−92 | −0.4157714 | 0.887 | 0.934 | 7.72E−88 |
| JUNB | 8.15E−90 | −0.558695 | 0.559 | 0.752 | 1.04E−85 |
| LGALS1 | 2.63E−81 | −0.7738655 | 0.287 | 0.508 | 3.37E−77 |
| HSPA1A | 3.22E−81 | −0.916248 | 0.105 | 0.305 | 4.12E−77 |
| CST7 | 1.21E−70 | 0.49913578 | 0.639 | 0.41 | 1.55E−66 |
| HLA-DQA1 | 5.44E−70 | 0.65204009 | 0.165 | 0.028 | 6.96E−66 |
| VIM | 7.15E−69 | −0.4924533 | 0.612 | 0.74 | 9.15E−65 |
| HAVCR2 | 2.33E−66 | 0.65234942 | 0.131 | 0.015 | 2.99E−62 |
| CD74 | 1.59E−63 | 0.58135286 | 0.661 | 0.495 | 2.03E−59 |
| HLA-DRA | 2.02E−62 | 0.65176928 | 0.324 | 0.14 | 2.59E−58 |
| LYST | 5.47E−62 | 0.57329667 | 0.255 | 0.087 | 7.00E−58 |
| COTL1 | 1.74E−61 | 0.47500207 | 0.63 | 0.432 | 2.23E−57 |
| HSPE1 | 1.29E−60 | −0.6207294 | 0.329 | 0.506 | 1.65E−56 |
| RPL12 | 3.26E−59 | 0.28766638 | 0.943 | 0.879 | 4.17E−55 |
| LAG3 | 4.65E−59 | 0.61757603 | 0.342 | 0.161 | 5.96E−55 |
| HSP90AB1 | 4.85E−58 | −0.4953474 | 0.516 | 0.658 | 6.20E−54 |
| RARRES3 | 2.77E−56 | 0.41873192 | 0.631 | 0.437 | 3.55E−52 |
| CAPG | 1.03E−55 | −0.5884383 | 0.069 | 0.218 | 1.32E−51 |
| CISH | 2.79E−55 | −0.6024594 | 0.07 | 0.218 | 3.58E−51 |
| FABP5 | 1.72E−54 | 0.64566009 | 0.282 | 0.12 | 2.20E−50 |
| IER2 | 1.24E−52 | −0.4978902 | 0.483 | 0.637 | 1.59E−48 |
| RNF19A | 7.79E−52 | 0.58149116 | 0.255 | 0.103 | 9.97E−48 |
| CXCR4 | 1.91E−51 | −0.5280569 | 0.485 | 0.639 | 2.45E−47 |
| CD27 | 6.36E−50 | 0.51126636 | 0.442 | 0.262 | 8.14E−46 |
| EIF1 | 1.34E−49 | −0.2535232 | 0.957 | 0.956 | 1.72E−45 |
| GNLY | 1.83E−49 | −0.8206381 | 0.104 | 0.258 | 2.34E−45 |
| RPL14 | 4.19E−49 | 0.24981354 | 0.943 | 0.874 | 5.36E−45 |
| IFI16 | 6.22E−49 | 0.4299199 | 0.555 | 0.37 | 7.96E−45 |
| PDCD1 | 2.77E−46 | 0.52705703 | 0.167 | 0.05 | 3.55E−42 |
| HLA-DQB1 | 5.11E−46 | 0.48883591 | 0.181 | 0.059 | 6.54E−42 |
| CD69 | 1.26E−44 | −0.4847954 | 0.457 | 0.611 | 1.61E−40 |
| STAT1 | 1.25E−43 | 0.51515463 | 0.3 | 0.151 | 1.59E−39 |
| KIAA1551 | 3.83E−43 | 0.45935097 | 0.339 | 0.182 | 4.90E−39 |
| SF3B1 | 7.69E−43 | 0.44397848 | 0.35 | 0.191 | 9.85E−39 |
| LTB | 1.66E−42 | −1.0005187 | 0.394 | 0.506 | 2.12E−38 |
| MT-ND3 | 1.67E−42 | −0.4511117 | 0.611 | 0.701 | 2.14E−38 |
| CD7 | 8.75E−42 | 0.48568723 | 0.521 | 0.361 | 1.12E−37 |
| CD38 | 9.62E−42 | 0.39235521 | 0.114 | 0.024 | 1.23E−37 |
| XCL1 | 1.54E−41 | −0.4822401 | 0.09 | 0.22 | 1.98E−37 |
| NFKBIA | 5.20E−41 | −0.5297075 | 0.272 | 0.429 | 6.66E−37 |
| UBC | 1.77E−40 | −0.2856539 | 0.743 | 0.822 | 2.26E−36 |
| PYHIN1 | 1.04E−39 | 0.42125928 | 0.372 | 0.208 | 1.33E−35 |
| MYL12A | 2.54E−39 | −0.2881323 | 0.78 | 0.82 | 3.25E−35 |
| TNFRSF9 | 6.52E−39 | 0.42931377 | 0.111 | 0.024 | 8.35E−35 |
| HLA-DPA1 | 1.22E−38 | 0.39223502 | 0.455 | 0.288 | 1.56E−34 |
| RGS16 | 2.38E−38 | −0.516725 | 0.029 | 0.12 | 3.04E−34 |
| GBP2 | 2.42E−38 | 0.42981859 | 0.275 | 0.136 | 3.10E−34 |
| BTG2 | 2.74E−38 | −0.5295653 | 0.185 | 0.328 | 3.51E−34 |
| PMAIP1 | 5.61E−38 | −0.4697427 | 0.128 | 0.262 | 7.18E−34 |
| IFNG | 1.05E−36 | 0.45363261 | 0.327 | 0.18 | 1.34E−32 |
| SLA | 1.26E−36 | 0.46160531 | 0.314 | 0.174 | 1.61E−32 |
| S100A10 | 8.16E−36 | −0.3864459 | 0.416 | 0.542 | 1.04E−31 |
| ITGB1 | 4.02E−35 | 0.48202857 | 0.237 | 0.117 | 5.15E−31 |
| RGS2 | 4.90E−35 | −0.4409712 | 0.221 | 0.366 | 6.27E−31 |
| NKG7 | 1.16E−34 | 0.4173697 | 0.723 | 0.625 | 1.48E−30 |
| SIRPG | 3.56E−34 | 0.45128694 | 0.202 | 0.09 | 4.56E−30 |
| FOS | 5.43E−34 | −0.2761692 | 0.446 | 0.598 | 6.95E−30 |
| ARAP2 | 1.30E−33 | 0.37715516 | 0.227 | 0.105 | 1.66E−29 |
| GBP5 | 3.12E−33 | 0.43391962 | 0.195 | 0.086 | 3.99E−29 |
| GIMAP7 | 6.46E−33 | 0.35199064 | 0.518 | 0.364 | 8.27E−29 |
| LBR | 9.65E−33 | 0.40079268 | 0.204 | 0.092 | 1.24E−28 |
| CD9 | 4.39E−32 | −0.4114685 | 0.027 | 0.106 | 5.61E−28 |
| HLA-DPB1 | 7.49E−32 | 0.33240097 | 0.513 | 0.36 | 9.59E−28 |
| NIN | 1.96E−31 | 0.3711078 | 0.144 | 0.052 | 2.51E−27 |
| DDX39B | 1.36E−30 | 0.3518629 | 0.1 | 0.026 | 1.74E−26 |
| SOD1 | 1.60E−30 | 0.35575111 | 0.531 | 0.401 | 2.04E−26 |
| IGLL5 | 3.18E−30 | −1.1318298 | 0.106 | 0.215 | 4.07E−26 |
| TOX | 8.71E−30 | 0.36416356 | 0.13 | 0.044 | 1.11E−25 |
| TIGIT | 9.83E−30 | 0.41085624 | 0.262 | 0.14 | 1.26E−25 |
| PARK7 | 1.01E−29 | 0.38128521 | 0.489 | 0.362 | 1.29E−25 |
| HIST1H1E | 1.31E−28 | 0.46541701 | 0.108 | 0.034 | 1.68E−24 |
| GZMA | 1.50E−28 | 0.25407975 | 0.824 | 0.709 | 1.92E−24 |
| 7-Sep | 2.94E−28 | 0.27044999 | 0.67 | 0.542 | 3.76E−24 |
| GADD45B | 3.57E−28 | −0.4535895 | 0.19 | 0.311 | 4.57E−24 |
| GZMK | 6.71E−28 | 0.37039626 | 0.609 | 0.483 | 8.59E−24 |
| ITM2A | 2.38E−27 | 0.36820652 | 0.514 | 0.385 | 3.05E−23 |
| LITAF | 3.23E−27 | 0.32587541 | 0.261 | 0.143 | 4.14E−23 |
| PTGER2 | 5.21E−27 | −0.3269009 | 0.037 | 0.115 | 6.67E−23 |
| PRMT10 | 2.15E−26 | −0.3805742 | 0.035 | 0.109 | 2.75E−22 |
| TRAT1 | 2.84E−26 | 0.36342233 | 0.277 | 0.161 | 3.64E−22 |
| SERTAD1 | 3.91E−26 | −0.4586827 | 0.101 | 0.199 | 5.01E−22 |
| HIST1H4C | 5.70E−26 | −0.3743404 | 0.173 | 0.29 | 7.29E−22 |
| XRN1 | 1.30E−25 | 0.32495918 | 0.183 | 0.088 | 1.66E−21 |
| RP11-51J9.5 | 1.54E−25 | −0.3776423 | 0.049 | 0.129 | 1.97E−21 |
| LINC00152 | 2.18E−25 | 0.31132281 | 0.342 | 0.215 | 2.79E−21 |
| IFRD1 | 7.81E−25 | −0.4200433 | 0.12 | 0.223 | 1.00E−20 |
| TRAF5 | 1.18E−24 | 0.32671157 | 0.149 | 0.065 | 1.51E−20 |
| SLC38A1 | 3.04E−24 | 0.3402513 | 0.193 | 0.099 | 3.90E−20 |
| CXCR6 | 4.63E−24 | 0.4087067 | 0.214 | 0.117 | 5.92E−20 |
| SRSF2 | 9.35E−24 | −0.3830785 | 0.348 | 0.453 | 1.20E−19 |
| FYN | 1.14E−23 | 0.31497937 | 0.268 | 0.158 | 1.46E−19 |
| SOCS1 | 1.24E−23 | −0.375926 | 0.086 | 0.176 | 1.59E−19 |
| ARL4A | 4.40E−23 | −0.4415337 | 0.076 | 0.159 | 5.63E−19 |
| GZMB | 6.52E−23 | 0.66674583 | 0.299 | 0.193 | 8.35E−19 |
| DNAJA1 | 6.95E−23 | −0.3706435 | 0.285 | 0.395 | 8.89E−19 |
| PRF1 | 7.00E−23 | 0.33372145 | 0.292 | 0.18 | 8.96E−19 |
| PLP2 | 1.01E−22 | −0.3633926 | 0.31 | 0.421 | 1.29E−18 |
| ATF7IP | 1.42E−22 | 0.28973378 | 0.142 | 0.062 | 1.82E−18 |
| ZFP36L2 | 1.43E−22 | −0.3620404 | 0.386 | 0.491 | 1.83E−18 |
| RGS1 | 1.92E−22 | −0.2406561 | 0.446 | 0.571 | 2.46E−18 |
| PERP | 2.37E−22 | −0.3393286 | 0.048 | 0.12 | 3.04E−18 |
| DDX17 | 4.76E−22 | 0.28382184 | 0.36 | 0.242 | 6.09E−18 |
| HLA-DMA | 9.90E−22 | 0.28277007 | 0.183 | 0.093 | 1.27E−17 |
| IFITM2 | 1.05E−21 | 0.29066227 | 0.505 | 0.382 | 1.35E−17 |
| KLRB1 | 1.23E−21 | −0.6574603 | 0.19 | 0.287 | 1.57E−17 |
| PTMS | 1.51E−21 | 0.32574418 | 0.162 | 0.08 | 1.93E−17 |
| PIK3R1 | 1.60E−21 | 0.31250329 | 0.22 | 0.125 | 2.05E−17 |
| EPB41 | 2.66E−21 | 0.27256347 | 0.104 | 0.039 | 3.40E−17 |
| ETS1 | 4.66E−21 | 0.31788565 | 0.205 | 0.115 | 5.96E−17 |
| CD84 | 6.68E−21 | 0.27319424 | 0.159 | 0.078 | 8.56E−17 |
| SETX | 7.34E−21 | 0.29481512 | 0.138 | 0.064 | 9.40E−17 |
| HEXIM1 | 8.26E−21 | −0.3822073 | 0.087 | 0.171 | 1.06E−16 |
| FAM65B | 1.55E−20 | 0.30502618 | 0.104 | 0.041 | 1.98E−16 |
| SYNE2 | 1.98E−20 | 0.33580569 | 0.323 | 0.22 | 2.53E−16 |
| HLA-F | 2.86E−20 | 0.25966556 | 0.368 | 0.254 | 3.66E−16 |
| ANXA1 | 6.35E−20 | −0.315856 | 0.355 | 0.46 | 8.13E−16 |
| GBP1 | 7.22E−20 | 0.32834772 | 0.211 | 0.123 | 9.24E−16 |
| ELF1 | 7.74E−20 | 0.26982387 | 0.348 | 0.237 | 9.90E−16 |
| HIF1A | 8.41E−20 | 0.29791757 | 0.118 | 0.052 | 1.08E−15 |
| DUSP2 | 1.05E−19 | −0.457562 | 0.342 | 0.433 | 1.34E−15 |
| GMFG | 1.72E−19 | 0.23812078 | 0.573 | 0.458 | 2.20E−15 |
| TUBA1B | 1.84E−19 | −0.2987512 | 0.349 | 0.462 | 2.36E−15 |
| LBH | 2.41E−19 | 0.2806684 | 0.29 | 0.187 | 3.09E−15 |
| ITGA4 | 2.58E−19 | 0.30357533 | 0.197 | 0.111 | 3.31E−15 |
| HIST1H1D | 2.91E−19 | 0.39830771 | 0.102 | 0.042 | 3.73E−15 |
| HSPA8 | 3.75E−19 | −0.2667883 | 0.607 | 0.652 | 4.80E−15 |
| C1orf162 | 3.89E−19 | −0.3013858 | 0.043 | 0.107 | 4.98E−15 |
| DNAJB9 | 4.12E−19 | −0.3450051 | 0.1 | 0.182 | 5.27E−15 |
| SRGN | 5.78E−19 | 0.26047884 | 0.744 | 0.667 | 7.40E−15 |
| LIMS1 | 6.35E−19 | 0.30370027 | 0.146 | 0.073 | 8.13E−15 |
| ID3 | 7.53E−19 | 0.35407484 | 0.137 | 0.066 | 9.64E−15 |
| RPS10 | 7.79E−19 | 0.22976722 | 0.589 | 0.472 | 9.97E−15 |
| PATL2 | 9.10E−19 | 0.30365265 | 0.105 | 0.043 | 1.16E−14 |
| ARID5B | 1.37E−18 | 0.30340745 | 0.125 | 0.058 | 1.75E−14 |
| TBCD | 1.73E−18 | 0.2699081 | 0.137 | 0.066 | 2.21E−14 |
| LDHA | 3.18E−18 | −0.3561296 | 0.52 | 0.581 | 4.07E−14 |
| CNOT6L | 3.19E−18 | 0.25897446 | 0.134 | 0.064 | 4.08E−14 |
| CBLB | 3.62E−18 | 0.2641167 | 0.115 | 0.051 | 4.64E−14 |
| EVI2B | 4.52E−18 | 0.249838 | 0.321 | 0.217 | 5.79E−14 |
| GIMAP4 | 6.11E−18 | 0.24476577 | 0.418 | 0.306 | 7.81E−14 |
| PAG1 | 8.89E−18 | 0.3033231 | 0.136 | 0.068 | 1.14E−13 |
| RPS27L | 1.42E−17 | −0.3472551 | 0.344 | 0.43 | 1.82E−13 |
| MIF | 1.54E−17 | −0.3311783 | 0.156 | 0.247 | 1.97E−13 |
| FLT3LG | 1.61E−17 | −0.2984567 | 0.055 | 0.119 | 2.06E−13 |
| YME1L1 | 1.86E−17 | −0.367169 | 0.142 | 0.225 | 2.38E−13 |
| VPS13C | 2.53E−17 | 0.27901639 | 0.163 | 0.089 | 3.24E−13 |
| LIMD2 | 3.49E−17 | 0.22347138 | 0.527 | 0.414 | 4.47E−13 |
| ENO1 | 3.80E−17 | −0.4131799 | 0.441 | 0.506 | 4.87E−13 |
| TAX1BP1 | 3.82E−17 | 0.22898117 | 0.368 | 0.259 | 4.89E−13 |
| HP1BP3 | 4.99E−17 | 0.23220628 | 0.246 | 0.156 | 6.38E−13 |
| CLEC2D | 5.03E−17 | 0.30587864 | 0.245 | 0.158 | 6.43E−13 |
| SLAMF7 | 5.52E−17 | 0.24856355 | 0.101 | 0.044 | 7.06E−13 |
| PPP1R15A | 7.90E−17 | −0.2922251 | 0.277 | 0.374 | 1.01E−12 |
| INPP4B | 1.00E−16 | 0.27195962 | 0.112 | 0.052 | 1.28E−12 |
| ACP5 | 1.28E−16 | 0.25354253 | 0.112 | 0.052 | 1.64E−12 |
| MIA3 | 1.33E−16 | 0.27480128 | 0.111 | 0.052 | 1.70E−12 |
| ILF3-AS1 | 1.64E−16 | −0.3615581 | 0.115 | 0.193 | 2.09E−12 |
| CD8A | 2.26E−16 | 0.22604718 | 0.51 | 0.403 | 2.90E−12 |
| RHOC | 3.60E−16 | −0.2954183 | 0.134 | 0.217 | 4.60E−12 |
| ZBTB38 | 3.63E−16 | 0.25284593 | 0.162 | 0.09 | 4.65E−12 |
| SMCHD1 | 3.94E−16 | 0.24053027 | 0.283 | 0.19 | 5.04E−12 |
| EMP3 | 4.48E−16 | −0.2426468 | 0.417 | 0.511 | 5.74E−12 |
| JAK1 | 4.90E−16 | 0.22338301 | 0.382 | 0.278 | 6.27E−12 |
| ITGAE | 5.67E−16 | 0.25408174 | 0.189 | 0.112 | 7.26E−12 |
| PAK2 | 6.10E−16 | 0.25111754 | 0.217 | 0.135 | 7.81E−12 |
| PDIA6 | 2.76E−15 | 0.24712246 | 0.299 | 0.21 | 3.54E−11 |
| GBP4 | 3.87E−15 | 0.2556482 | 0.121 | 0.061 | 4.95E−11 |
| NUCB2 | 3.97E−15 | 0.23763916 | 0.218 | 0.138 | 5.08E−11 |
| ASXL2 | 4.24E−15 | 0.27516671 | 0.102 | 0.048 | 5.43E−11 |
| SYNRG | 7.68E−15 | 0.22825636 | 0.208 | 0.129 | 9.83E−11 |
| FUBP1 | 9.42E−15 | 0.23586111 | 0.161 | 0.092 | 1.21E−10 |
| ZNF644 | 9.92E−15 | 0.23258407 | 0.153 | 0.086 | 1.27E−10 |
| SLC2A3 | 2.32E−14 | −0.329732 | 0.129 | 0.203 | 2.96E−10 |
| CLIC3 | 2.37E−14 | 0.3021681 | 0.135 | 0.073 | 3.03E−10 |
| TMPO | 3.13E−14 | 0.23394776 | 0.125 | 0.066 | 4.00E−10 |
| DDX24 | 3.25E−14 | −0.2600534 | 0.423 | 0.503 | 4.16E−10 |
| CLK1 | 3.41E−14 | −0.3189956 | 0.244 | 0.325 | 4.36E−10 |
| DCP2 | 3.45E−14 | 0.2251654 | 0.132 | 0.071 | 4.42E−10 |
| ZFP36 | 3.55E−14 | −0.2890474 | 0.308 | 0.393 | 4.54E−10 |
| BATF | 5.05E−14 | 0.2926783 | 0.184 | 0.115 | 6.47E−10 |
| PTP4A1 | 6.88E−14 | −0.2839973 | 0.059 | 0.115 | 8.80E−10 |
| FKBP5 | 1.02E−13 | 0.23236769 | 0.112 | 0.058 | 1.30E−09 |
| C9orf16 | 1.10E−13 | 0.23552898 | 0.357 | 0.27 | 1.40E−09 |
| FOSB | 1.14E−13 | −0.3582543 | 0.164 | 0.237 | 1.45E−09 |
| SEMA4D | 1.58E−13 | 0.23997496 | 0.125 | 0.068 | 2.03E−09 |
| P2RY10 | 1.96E−13 | 0.22782395 | 0.1 | 0.049 | 2.50E−09 |
| LSM2 | 2.03E−13 | 0.23295584 | 0.221 | 0.147 | 2.60E−09 |
| SMARCA5 | 2.43E−13 | 0.24114457 | 0.199 | 0.128 | 3.11E−09 |
| RP11-94L15.2 | 2.81E−13 | 0.2482197 | 0.155 | 0.092 | 3.60E−09 |
| POLR2L | 2.93E−13 | −0.2567805 | 0.301 | 0.383 | 3.75E−09 |
| C1orf56 | 3.20E−13 | −0.3368884 | 0.081 | 0.141 | 4.10E−09 |
| KLRD1 | 6.41E−13 | 0.31371177 | 0.199 | 0.13 | 8.20E−09 |
| IVNS1ABP | 7.40E−13 | −0.2460142 | 0.049 | 0.1 | 9.47E−09 |
| TXNIP | 8.73E−13 | 0.27114818 | 0.566 | 0.492 | 1.12E−08 |
| DDIT3 | 9.05E−13 | −0.2957799 | 0.052 | 0.102 | 1.16E−08 |
| TNFRSF1B | 1.52E−12 | 0.25673538 | 0.161 | 0.099 | 1.94E−08 |
| SNHG8 | 1.56E−12 | −0.3243208 | 0.162 | 0.232 | 1.99E−08 |
| SBDS | 2.38E−12 | −0.297562 | 0.152 | 0.221 | 3.05E−08 |
| SAMD9 | 2.60E−12 | 0.24061015 | 0.268 | 0.19 | 3.33E−08 |
| CLDND1 | 3.11E−12 | −0.3134506 | 0.107 | 0.169 | 3.98E−08 |
| EIF1AX | 3.15E−12 | −0.3000081 | 0.117 | 0.182 | 4.03E−08 |
| ARF6 | 6.06E−12 | −0.29755 | 0.232 | 0.305 | 7.76E−08 |
| CCNK | 1.17E−11 | 0.22605602 | 0.106 | 0.058 | 1.49E−07 |
| RNASET2 | 1.53E−11 | 0.24375783 | 0.203 | 0.137 | 1.96E−07 |
| CKLF | 2.29E−11 | −0.2419013 | 0.482 | 0.534 | 2.94E−07 |
| CHMP1B | 2.41E−11 | −0.3176326 | 0.136 | 0.198 | 3.08E−07 |
| PIM1 | 2.95E−11 | −0.2720632 | 0.153 | 0.221 | 3.77E−07 |
| ANXA2 | 4.51E−11 | −0.2548039 | 0.245 | 0.32 | 5.78E−07 |
| RTN4 | 5.82E−11 | −0.269316 | 0.172 | 0.24 | 7.45E−07 |
| TNF | 6.19E−11 | −0.2861679 | 0.098 | 0.155 | 7.93E−07 |
| CRIP1 | 9.96E−11 | −0.2745251 | 0.128 | 0.189 | 1.28E−06 |
| CTSA | 1.17E−10 | −0.2729227 | 0.131 | 0.193 | 1.50E−06 |
| EIF5A | 1.56E−10 | −0.2573108 | 0.233 | 0.305 | 2.00E−06 |
| SYPL1 | 2.08E−10 | −0.2282569 | 0.077 | 0.129 | 2.67E−06 |
| CSRNP1 | 2.35E−10 | −0.2540597 | 0.059 | 0.105 | 3.00E−06 |
| CTSS | 2.76E−10 | 0.22337202 | 0.181 | 0.124 | 3.53E−06 |
| VAMP2 | 3.25E−10 | −0.2556979 | 0.273 | 0.341 | 4.16E−06 |
| TSPYL2 | 3.86E−10 | −0.2759328 | 0.114 | 0.17 | 4.94E−06 |
| FAM3C | 4.47E−10 | 0.2380999 | 0.103 | 0.059 | 5.72E−06 |
| RILPL2 | 4.78E−10 | 0.24069741 | 0.138 | 0.086 | 6.12E−06 |
| JUND | 5.97E−10 | −0.2499589 | 0.071 | 0.118 | 7.64E−06 |
| TPI1 | 7.06E−10 | −0.3230395 | 0.442 | 0.487 | 9.03E−06 |
| SAT1 | 1.11E−09 | −0.3020162 | 0.379 | 0.435 | 1.42E−05 |
| TUBB4B | 1.34E−09 | −0.2457038 | 0.141 | 0.202 | 1.72E−05 |
| RUNX3 | 1.51E−09 | −0.248478 | 0.139 | 0.198 | 1.93E−05 |
| YPEL5 | 3.81E−09 | −0.3083842 | 0.202 | 0.263 | 4.88E−05 |
| C1orf63 | 3.93E−09 | −0.2478256 | 0.407 | 0.463 | 5.03E−05 |
| C8orf59 | 6.20E−09 | −0.2437501 | 0.135 | 0.19 | 7.94E−05 |
| SLC38A2 | 8.03E−09 | −0.2674882 | 0.104 | 0.154 | 0.00010273 |
| USMG5 | 9.86E−09 | −0.2332556 | 0.321 | 0.376 | 0.00012619 |
| POLR2J3 | 1.04E−08 | −0.2372979 | 0.107 | 0.158 | 0.00013354 |
| IDS | 1.23E−08 | −0.2588677 | 0.218 | 0.278 | 0.00015767 |
| RP11-489E7.4 | 1.28E−08 | −0.2709839 | 0.075 | 0.119 | 0.00016373 |
| PGK1 | 1.66E−08 | −0.2681581 | 0.351 | 0.399 | 0.00021199 |
| TAF7 | 1.93E−08 | −0.2426841 | 0.318 | 0.373 | 0.00024707 |
| TLE4 | 2.39E−08 | −0.278814 | 0.131 | 0.182 | 0.00030616 |
| HIST1H1C | 3.56E−08 | 0.31983309 | 0.124 | 0.082 | 0.00045501 |
| RP11-640M9.1 | 3.81E−08 | −0.2271791 | 0.089 | 0.135 | 0.00048738 |
| TMEM107 | 4.02E−08 | −0.2376275 | 0.073 | 0.114 | 0.00051444 |
| GRAP2 | 6.88E−08 | −0.2282248 | 0.12 | 0.17 | 0.00088015 |
| CCL3 | 1.56E−07 | 0.40554359 | 0.207 | 0.156 | 0.00199384 |
| CCL4 | 2.80E−07 | 0.32767617 | 0.556 | 0.481 | 0.00358535 |
| HERPUD1 | 3.24E−07 | −0.2521958 | 0.189 | 0.239 | 0.00415142 |
| WTAP | 5.37E−07 | −0.2426846 | 0.156 | 0.203 | 0.00687018 |
| NR4A2 | 5.89E−07 | −0.2258402 | 0.12 | 0.165 | 0.00753413 |
| AMICA1 | 6.16E−07 | 0.234881 | 0.209 | 0.164 | 0.00787931 |
| PTP4A2 | 6.78E−07 | −0.230104 | 0.219 | 0.268 | 0.00867857 |
| TUBA1A | 7.98E−07 | −0.2771119 | 0.23 | 0.278 | 0.01021962 |
| CITED2 | 2.40E−06 | −0.2398126 | 0.181 | 0.227 | 0.0307365 |

TABLE 5
Directionality of Change of Activity, illustrated in FIG. 4E

| Transcription Factors | Activity |
|---|---|
| MYC | UP |
| CUX1 | UP |
| TAF1 | UP |
| SPI1 | UP |
| EGR1 | DOWN |
| ESR1 | DOWN |
| NR4A1 | DOWN |
| GTF2B | DOWN |
| TCF4 | DOWN |
| RAD21 | DOWN |
| AR | DOWN |
| EP300 | DOWN |
| TP53 | DOWN |
| NR3C1 | DOWN |
| IRF6 | DOWN |
| GATA1 | DOWN |
| SMAD3 | DOWN |
| TFAP2A | DOWN |
| JUN | DOWN |
| JUND | DOWN |

The presence or quantity of biomarker(s) or panels of markers, as indicated herein for particular markers, can be assessed by comparing a value to a relevant reference level. For example, the quantity of one or more markers can be indicated as a value. The value can be one or more numerical values resulting from the assaying of a sample, and can be derived, e.g., by measuring level(s) of the marker(s) in the sample by an assay performed in a laboratory, or from a dataset obtained from a provider such as a laboratory, or from a dataset stored on a server. The markers disclosed herein in many embodiments are protein marker(s), though nucleic acid markers (for instance, a gene encoding a protein marker) are also contemplated.

In the broadest sense, the value may be qualitative or quantitative. As such, where detection is qualitative, the systems and methods provide a reading or evaluation, e.g., assessment, of whether or not the marker is present in the sample being assayed. In yet other embodiments, the systems and methods provide a quantitative detection of whether the marker is present in the sample being assayed, i.e., an evaluation or assessment of the actual amount or relative abundance of the marker in the sample being assayed. In such embodiments, the quantitative detection may be absolute or, if the method is a method of detecting two or more different markers in a sample, relative. As such, the term “quantifying” when used in the context of quantifying a marker in a sample can refer to absolute or to relative quantification; it is recognized that the quantity of a marker can be used as correlative to the number of cells in/on which that marker is expressed. Thus, quantification may simply refer to counting the number of cells that are labeled with an agent that binds specifically to a marker expressed on/in or associated with that cell type.

Absolute quantification can be accomplished by inclusion of known concentration(s) of one or more control markers and referencing, e.g., normalizing, the detected level of the marker with the known control markers (e.g., through generation of a standard curve). Alternatively, relative quantification can be accomplished by comparison of detected levels or amounts between two or more different markers to provide a relative quantification of each of the two or more markers, e.g., relative to each other. The actual measurement of values of the markers can be determined at the protein or nucleic acid level using any method known in the art. In some embodiments, a marker is detected by contacting a sample with reagents (e.g., antibodies or nucleic acid primers), generating complexes of reagent and marker(s), and detecting the complexes.

The reagent can include a probe. A probe is a molecule that binds a target, either directly or indirectly. The target can be a marker, a fragment of the marker, or any molecule that is to be detected. In embodiments, the probe includes a nucleic acid or a protein. As an example, a protein probe can be an antibody. An antibody can be a whole antibody or a fragment of an antibody. A probe can be labeled with a detectable label. Examples of detectable labels include fluorescers, chemiluminescers, dyes, enzymes, enzyme substrates, enzyme cofactors, enzyme inhibitors, enzyme subunits, metal ions, and radioactive isotopes.

“Protein” detection includes detection of full-length proteins, mature proteins, pre-proteins, polypeptides, isoforms, mutations, post-translationally modified proteins and variants thereof, and can be detected in any suitable manner.

Those skilled in the art will be familiar with numerous specific immunoassay formats and variations thereof which can be useful for carrying out the methods disclosed herein. See, e.g., E. Maggio, Enzyme-Immunoassay (1980), CRC Press, Inc., Boca Raton, Fla; and U.S. Pat. Nos. 4,727,022; 4,659,678; 4,376,110; 4,275,149; 4,233,402; and 4,230,797.

Antibodies can be conjugated to a solid support suitable for a diagnostic assay (e.g., beads such as protein A or protein G agarose, microspheres, plates, slides or wells formed from materials such as latex or polystyrene) in accordance with known techniques, such as passive binding. Antibodies can be conjugated to detectable labels or groups such as radiolabels (e.g., ³⁵S, ¹²⁵I, ¹³¹I), enzyme labels (e.g., horseradish peroxidase, alkaline phosphatase), and fluorescent labels (e.g., fluorescein, Alexa, green fluorescent protein, rhodamine) in accordance with known techniques.

Examples of suitable immunoassays include immunoblotting, immunoprecipitation, immunofluorescence, chemiluminescence, electro-chemiluminescence (ECL), and/or enzyme-linked immunoassays (ELISA).

Antibodies may also be useful for detecting post-translational modifications of markers. Examples of post-translational modifications include tyrosine phosphorylation, threonine phosphorylation, serine phosphorylation, citrullination, and glycosylation (e.g., O-GIcNAc). Such antibodies specifically detect the phosphorylated amino acids in marker proteins of interest. These antibodies are well-known to those skilled in the art, and commercially available. Post-translational modifications can also be determined using metastable ions in reflector matrix-assisted laser desorption ionization-time of flight mass spectrometry (MALDI-TOF). See Wirth et al., Proteomics 2002, 2(10):1445-1451.

Up- or down-regulation of genes (which are a type of marker) also can be detected using, for example, cDNA arrays, cDNA fragment fingerprinting, cDNA sequencing, clone hybridization, differential display, differential screening, FRET detection, liquid microarrays, PCR, RT-PCR, quantitative real-time RT-PCR analysis with TaqMan assays, molecular beacons, microelectric arrays, oligonucleotide arrays, polynucleotide arrays, serial analysis of gene expression (SAGE), and/or subtractive hybridization.

As an example, Northern hybridization analysis using probes that specifically recognize one or more marker sequences can be used to determine gene expression. Alternatively, expression can be measured using RT-PCR; e.g., polynucleotide primers specific for the differentially expressed marker mRNA sequences reverse-transcribe the mRNA into DNA, which is then amplified in PCR and can be visualized and quantified. Marker RNA can also be quantified using, for example, other target amplification methods, such as transcription mediated amplification (TMA), strand displacement amplification (SDA), and nucleic acid sequence based amplification (NASBA), or signal amplification methods (e.g., bDNA), and the like. Ribonuclease protection assays can also be used, using probes that specifically recognize one or more marker mRNA sequences, to determine gene expression.

Further hybridization technologies that may be used are described in, for example, U.S. Pat. Nos. 5,143,854; 5,288,644; 5,324,633; 5,432,049; 5,470,710; 5,492,806; 5,503,980; 5,510,270; 5,525,464; 5,547,839; 5,580,732; 5,661,028; and 5,800,992 as well as WO 95/21265; WO 96/31622; WO 97/10365; WO 97/27317; EP 373 203; and EP 785 280.

Proteins and nucleic acids can be linked to chips, such as microarray chips. See, for example, U.S. Pat. Nos. 5,143,854; 6,087,112; 5,215,882; 5,707,807; 5,807,522; 5,958,342; 5,994,076; 6,004,755; 6,048,695; 6,060,240; 6,090,556; and 6,040,138. Microarray refers to a solid carrier or support that has a plurality of molecules bound to its surface at defined locations. The solid carrier or support can be made of any material. As an example, the material can be hard, such as metal, glass, plastic, silicon, ceramics, and textured and porous materials; or soft materials, such as gels, rubbers, polymers, and other non-rigid materials. The material can also be nylon membranes, epoxy-glass and borofluorate-glass. The solid carrier or support can be flat, but need not be and can include any type of shape such as spherical shapes (e.g., beads or microspheres). The solid carrier or support can have a flat surface as in slides and micro-titer plates having one or more wells.

Binding to proteins or nucleic acids on microarrays can be detected by scanning the microarray with a variety of laser or CCD-based scanners, and extracting features with software packages, for example, Imagene (Biodiscovery, Hawthorne, CA), Feature Extraction Software (Agilent), Scanalyze (Eisen, M. 1999. SCANALYZE User Manual; Stanford Univ., Stanford, CA Ver 2.32.), or GenePix (Axon Instruments).

Embodiments disclosed herein can be used with high throughput screening (HTS). Typically, HTS refers to a format that performs at least 100 assays, at least 500 assays, at least 1000 assays, at least 5000 assays, at least 10,000 assays, or more per day. When enumerating assays, either the number of samples or the number of protein (or nucleic acid) biomarkers assayed can be considered.

Generally HTS methods involve a logical or physical array of either the subject samples, or the protein or nucleic acid markers, or both. Appropriate array formats include both liquid and solid phase arrays. For example, assays employing liquid phase arrays, e.g., for hybridization of nucleic acids, binding of antibodies or other receptors to ligand, etc., can be performed in multiwell or microtiter plates. Microtiter plates with 96, 384, or 1536 wells are widely available, and even higher numbers of wells, e.g., 3456 and 9600 can be used. In general, the choice of microtiter plates is determined by the methods and equipment, e.g., robotic handling and loading systems, used for sample preparation and analysis.

HTS assays and screening systems are commercially available from, for example, Zymark Corp. (Hopkinton, MA); Air Technical Industries (Mentor, OH); Beckman Instruments, Inc. (Fullerton, CA); Precision Systems, Inc. (Natick, MA), and so forth. These systems typically automate entire procedures including all sample and reagent pipetting, liquid dispensing, timed incubations, and final readings of the microplate in detector(s) appropriate for the assay. These configurable systems provide HTS as well as a high degree of flexibility and customization. The manufacturers of such systems provide detailed protocols for the various methods of HTS.

As stated previously, obtained marker values can be compared to a reference level. Reference levels can be obtained from one or more relevant datasets. A “dataset” as used herein is a set of numerical values resulting from evaluation of a sample (or population of samples) under a desired condition. The values of the dataset can be obtained, for example, by experimentally obtaining measures from a sample and constructing a dataset from these measurements. As is understood by one of ordinary skill in the art, the reference level can be based on e.g., any mathematical or statistical formula useful and known in the art for arriving at a meaningful aggregate reference level from a collection of individual datapoints; e.g., mean, median, median of the mean, and so forth. Alternatively, a reference level or dataset to create a reference level can be obtained from a service provider such as a laboratory, or from a database or a server on which the dataset has been stored.

A reference level from a dataset can be derived from previous measures derived from a population. A “population” is any grouping of subjects or samples of like specified characteristics. The grouping could be according to, for example, clinical parameters, clinical assessments, therapeutic regimens, disease status, severity of condition, etc.

Subjects include humans, veterinary animals (dogs, cats, reptiles, birds, hamsters, etc.) livestock (horses, cattle, goats, pigs, chickens, etc.), research animals (monkeys, rats, mice, fish, etc.) and other animals, such as zoo animals (e.g., bears, giraffe, elephant, lemurs, etc.).

In particular embodiments, conclusions are drawn based on whether a sample value is statistically significantly different or not statistically significantly different from a reference level. A measure is not statistically significantly different if the difference is within a level that would be expected to occur based on chance alone. In contrast, a statistically significant difference or increase is one that is greater than what would be expected to occur by chance alone. Statistical significance or lack thereof can be determined by any of various methods well-known in the art. An example of a commonly used measure of statistical significance is the p-value. The p-value represents the probability of obtaining a given result equivalent to a particular datapoint, where the datapoint is the result of random chance alone. A result is often considered statistically significant (not the result of random chance) at a p-value less than or equal to 0.05.

In one embodiment, values obtained about the markers and/or other dataset components can be subjected to an analytic process with chosen parameters. The parameters of the analytic process may be those disclosed herein or those derived using the guidelines described herein. The analytic process used to generate a result may be any type of process capable of providing a result useful for classifying a sample, for example, comparison of the obtained value with a reference level, a linear algorithm, a quadratic algorithm, a decision tree algorithm, or a voting algorithm. The analytic process may set a threshold for determining the probability that a sample belongs to a given class. The probability preferably is at least at least 60%, at least 70%, at least 80%, at least 90%, at least 95% or higher.

In embodiments, the relevant reference level for a particular marker is obtained based on the particular marker in control subjects. Control subjects are those that are healthy and do not have the pathology being assayed, for instance not having the target inflammatory condition or the target cancer. As an example, the relevant reference level can be the quantity of the particular biomarker in control subject(s).

### Kits

Kits, including at least one therapeutic or diagnostic component, are also provided. Such kits can include at least one compound that reduces the expression, level, function, and/or activity of androgen receptor (examples of which are described herein); optionally along with at least one agent effective in checkpoint therapy/blockade (e.g., a checkpoint inhibitor, such as an inhibitor of PD-1, PD-L1, Tim3, Lag3, TIGIT, or CTLA4); and/or an epigenetic modifier (e.g., an EZH2 inhibitor or LAD1 inhibitor); and/or an immunotherapeutic agent; diluents to reconstitute for injection, and appropriate buffers. Kits can alternatively or additionally include one or more arrays that enable detection of one or more genes/sequences included in a biomarker described herein. In particular embodiments, kits can also include some or all of the necessary laboratory and/or medical supplies needed to use the kit effectively, such as gauze, sterile adhesive strips, gloves, tubes, and the like. Variations in contents of any of the kits described herein can be made.

Compounds and/or compositions required to carry out a method described herein, for instance one or more antibodies (or other detection molecules) specific for a biomarker, or a panel of biomarkers as discussed herein, can be provided as kits. Kits can include one or more containers including (containing) one or more or more detection or other compounds as described herein, optionally along with one or more agents for use in sample analysis and/or one or more agents for use in therapy. For instance, some kits will include an amount of at least one anti-cancer composition.

Any component in a kit may be provided in premeasured dosage(s), though this is not required; and it is expected that example kits will include more than one dose.

Embodiments of kits can contain, in separate containers, marker binding agents either bound to a matrix, or packaged separately with reagents for binding to a matrix. In particular embodiments, the matrix is, for example, a porous strip. In some embodiments, measurement or detection regions of the porous strip can include a plurality of sites containing marker binding agents. In some embodiments, the porous strip can also contain sites for negative and/or positive controls. Alternatively, control sites can be located on a separate strip from the porous strip. Optionally, the different detection sites can contain different amounts of marker binding agents, e.g., a higher amount in the first detection site and lesser amounts in subsequent sites. Upon the addition of test sample, the number of sites displaying a detectable signal provides a quantitative indication of the amount of marker present in the sample. The detection sites can be configured in any suitably detectable shape and can be, e.g., in the shape of a bar or dot spanning the width (or a portion thereof) of a porous strip.

In some embodiments the matrix can be a solid substrate, such as a “chip.” See, e.g., U.S. Pat. No. 5,744,305. In some embodiments the matrix can be a solution array; e.g., xMAP (Luminex, Austin, TX), Cyvera (Illumina, San Diego, CA), RayBio Antibody Arrays (RayBiotech, Inc., Norcross, GA), CellCard (Vitra Bioscience, Mountain View, CA) and Quantum Dots' Mosaic (Invitrogen, Carlsbad, CA).

Additional embodiments can include control formulations (positive and/or negative), and/or one or more detectable labels, such as fluorescein, green fluorescent protein, rhodamine, cyanine dyes, Alexa dyes, luciferase, and radiolabels, among others. Instructions for carrying out the assay, including, optionally, instructions for generating a score, can be included in the kit; e.g., written, tape, VCR, or CD-ROM.

In particular embodiments, the kits include materials and reagents necessary to conduct and immunoassay (e.g., ELISA). In particular embodiments, the kits include materials and reagents necessary to conduct hybridization assays (e.g., PCR). In particular embodiments, materials and reagents expressly exclude equipment (e.g., plate readers). In particular embodiments, kits can exclude materials and reagents commonly found in laboratory settings (pipettes; test tubes; distilled H₂O).

Components of the kit can be prepared for storage and later use. Associated with such container(s) can be noticed in the form prescribed by a governmental agency regulating the manufacture, use, or sale of the kit, which notice reflects approval by the agency of manufacture, use, or sale when required.

Kits can also include a notice in the form prescribed by a governmental agency regulating the manufacture, use, or sale of pharmaceuticals or biological products, which notice reflects approval by the agency of manufacture, use, or sale for human administration. The notice may state that the provided active ingredients can be administered to a subject. The kits can include further instructions for using the kit, for example, instructions regarding administration; proper disposal of related waste; and the like. The instructions can be in the form of printed instructions provided within the kit or the instructions can be printed on a portion of the kit itself. Instructions may be in the form of a sheet, pamphlet, brochure, CD-ROM, or computer-readable device, or can provide directions to instructions at a remote location, such as a website. In particular embodiments, kits can also include some or all of the necessary medical supplies needed to use the kit effectively, such as applicators, ampules, sponges, sterile adhesive strips, Chloraprep, gloves, and the like. Variations in contents of any of the kits described herein can be made. The instructions of the kit will direct use of the active ingredient(s) included in that kit to effectuate a clinical and/or therapeutic use described herein.

Optionally, the kits further include instructions for using the kit in the methods. In various embodiments, the instructions can include appropriate instructions to interpret results associated with using the kit, proper disposal of the related waste, and the like. The instructions can be in the form of printed instructions provided within the kit, or the instructions can be printed on a portion of the kit itself. Instructions may be in the form of a sheet, pamphlet, brochure, CD-ROM, or computer-readable device, or can provide directions to instructions at a remote location, such as a website.

Exemplary Embodiments and Example(s) included herein or in any of the herewith submitted documents are included to demonstrate particular embodiments of the disclosure. Those of ordinary skill in the art should recognize in light of the present disclosure that many changes can be made to the specific embodiments disclosed herein and still obtain a like or similar result without departing from the spirit and scope of the disclosure.

### Exemplary Embodiments

1. A method of improving function of an immune cell or population of immune cells, including reducing intrinsic androgen receptor (AR) expression or activity level.
2. The method of embodiment 1, wherein the immune cell or a cell within the population of immune cells is: a T-cell, a myeloid cell, a macrophage, a dendritic cell, a natural killer cell, or a B-cell.
3. The method of embodiment 1, wherein the cell is a T cell, and reducing the intrinsic AR level improves T cell function in allogenic T cell therapy, adoptive cell therapy, chimeric antigen receptor engineered T (CAR-T) cell therapy, or autologous T cell therapy.
4. The method of embodiment 1, wherein the cell is a T cell, and reducing the intrinsic AR level reduces regulatory T cell suppression.
5. The method of embodiment 1, wherein the cell is a myeloid cell or a macrophage, and reducing the intrinsic AR level improves engineered macrophage therapy.
6. The method of embodiment 1, wherein the cell is a dendritic cell, and reducing the intrinsic AR level improves antigen presentation and dendritic cell vaccine therapy.
7. The method of embodiment 1, wherein the cell is a natural killer cell, and reducing the intrinsic AR level improves cell killing.
8. The method of embodiment 1, wherein the cell is a B cell, and reducing the intrinsic AR level improves antibody maturation and vaccination.
9. An immune therapeutic cell genetically modified to reduce expression of intrinsic androgen receptor (AR).
10. The cell of embodiment 9, wherein the cell is a T-cell, a myeloid cell, a macrophage, a dendritic cell, a natural killer cell, or a B-cell.
11. A cell formulation including a cell of embodiment 9 or embodiment 10 and a pharmaceutically acceptable carrier.
12. A method of genetically-modifying an immune cell to provide an improved immune therapy product, the method including inhibiting expression and/or activity of androgen receptor (AR), in the immune cell.
13. The method of embodiment 12, wherein the method includes inhibiting expression and/or activity of androgen receptor (AR) in at least one cell other than the immune cell.
14. A method of overcoming inhibition or failure of an immunotherapy response, including reducing expression and/or activity of androgen receptor (AR) in a cell used in the immunotherapy.
15. A method of improving the therapeutic effectiveness of an immunotherapy, including reducing expression and/or activity of androgen receptor (AR) in a cell used in the immunotherapy.
16. The method of any one of embodiments 12-15, wherein reducing expression and/or activity of androgen receptor (AR) in the cell includes one or more of: modification of a genomic sequence encoding AR, such as through targeted gene editing/disruption; post-transcriptional inhibition of AR expression, such as through induction of RNAi; and/or pharmacological inhibition or degradation of AR activity or function.
17. The method of embodiment 16, wherein the targeted gene editing includes Clustered Regularly Interspaced Short Palindromic Repeats/CRISPR-associated (CRISPR/Cas) nuclease editing of a genomic sequence encoding AR, zinc finger nuclease (ZFN) editing of a genomic sequence encoding AR, or transcription activator like effector nuclease (TALEN) editing of a genomic sequence encoding AR.
18. A method of improving efficacy of immune checkpoint blockade therapy, including conducting the immune checkpoint blockade therapy (1) with a genetically-modifying cell in which the genetic modification results in reduction in androgen receptor (AR) expression and/or activity, or (2) in the presence of an AR agonist or antagonist.
19. The method of embodiment 18, wherein the immune checkpoint blockade therapy includes cancer treatment.
20. The method of embodiment 19, wherein the immune checkpoint blockade therapy includes treatment of prostate cancer or sarcoma.
21. Use of an immune cell engineered to have reduced androgen receptor (AR) expression and/or activity to treat cancer.
22. The use of embodiment 21, wherein the cancer includes prostate cancer or sarcoma.
23. The use of embodiment 21 or embodiment 22, wherein the reduced AR expression and/or activity in the cell is accomplished through a method includes one or more of: modification of a genomic sequence encoding AR, such as through targeted gene editing/disruption; post-transcriptional inhibition of AR expression, such as through induction of RNAi or ubiquitin mediated degradation; and/or pharmacological inhibition of AR activity or function.
24. The use of embodiment 23, wherein the targeted gene editing includes Clustered Regularly Interspaced Short Palindromic Repeats/CRISPR-associated (CRISPR/Cas) nuclease editing of a genomic sequence encoding AR, zinc finger nuclease (ZFN) editing of a genomic sequence encoding AR, or transcription activator like effector nuclease (TALEN) editing of a genomic sequence encoding AR.
25. A method, including: dual inhibition of AR and an immune checkpoint, to reduce tumor growth and improve survival; treatment with androgen deprivation therapy (ADT), AR inhibition (e.g., with enzalutamide, bicalutamide, flutamide, abiraterone, or the like) or AR degradation (e.g., with AR-PROTACs), and immune checkpoint blockade (such as PD-1 blockade) in a subject that has become refractory to checkpoint therapy; or increasing the effectiveness of checkpoint blockade (such as PD-1 blockade, TIGIT blockade, TIM3 blockade, CTLA4 blockade, and so forth), by reducing AR activity in CD8 T cells in patients refractory to checkpoint therapy.
26. A method to increase production of effector cytokines (such as granzyme B, IFNγ and/or TNFα) in a subject, including treating the subject with: an androgen reduction therapy (such as ADT, an AR inhibitor, an AR degrader, or a combination thereof); and an anti-PD-L1 agent or other agent that provides (immune) checkpoint blockade.
27. The method of embodiment 26, wherein the androgen reduction therapy includes treatment with a luteinizing hormone-releasing hormone (LHRH) inhibitor (such as degarelix) or orchiectomy.
28. A method to increase effectiveness of immune therapies and immune-mediated tumor including treating a subject with a combination of AR inhibitor with an epigenetic modifier (such as an EZH2 inhibitor or LSD1 inhibitor), alone or with immunotherapy.
29. A method, including treating a subject with an AR inhibitor, an epigenetic modifier, and immune cell therapy (such as autologous immune cell transfer).
30. A biomarker allowing identification of patients who could achieve a clinical benefit from checkpoint blockade, or to distinguish responsive from non-responsive patients, essentially as described herein.
31. The biomarker of embodiment 30, wherein the checkpoint blockade includes PD-L1 blockade.
32. The biomarker of embodiment 30, wherein the biomarker includes: one or more of immune cell intrinsic AR activity, or CD8_R signature, or IFNG pathway activity.
33. The biomarker of embodiment 30, wherein the biomarker includes expression level of at least one gene that is up-regulated in AR KO T cells, and/or expression level of at least one gene that is described herein as down-regulated in AR KO T cells.
34. The biomarker of embodiment 33, wherein the biomarker includes expression level of at least two genes, at least three genes, at least four genes, at least five genes, at least six genes, at least seven genes, at least eight genes, at least nine genes, or more than nine genes that are up-regulated in AR KO T cells, and/or expression level of at least two genes, at least three genes, at least four genes, at least five genes, at least six genes, at least seven genes, at least eight genes, at least nine genes, or more than nine genes that are described herein as down-regulated in AR KO T cells.
35. The biomarker of embodiment 33 or embodiment 34, wherein: the gene(s) up-regulated in AR KO T cells are selected from the group consisting of H2-Eb1, H2-Aa, Il13, Igfbp4, Rsad2, Rpl28-ps1, Gm1821, Tmem106a, Rpl36a-ps2, Ceacam-ps1, Gpr15, Trp53cor1, Rnaset2a, Gm10288, AL607105.1, and Emp1; or the gene(s) down-regulated in AR KO T cells are selected from the group consisting of Traj24, Tm4sf19, Snord32a, AC159308.5, Gm15564, Gm36931, Gm45599, Gm6939, Gm26642, Arg1, Cxcl3, Gm590, Sema6b, 5830411N06Rik, Gm37768, Traj11, Gm38319, AC133083.2, Traj22, Gm45803, Gm17606, Klhl35, Gm26835, AC132474.1, Ankrd55, Ccl22, Gm37105 and Il10; or both.
36. The biomarker of embodiment 30, including: expression level of H2-Eb1, H2-Aa, Il13, Igfbp4, Rsad2, Rpl28-ps1, Gm1821, Tmem106a, Rp136a-ps2, Ceacam-ps1, Gpr15, Trp53cor1, Rnaset2a, Gm10288, AL607105.1, and Emp1; or expression level of Traj24, Tm4sf19, Snord32a, AC159308.5, Gm15564, Gm36931, Gm45599, Gm6939, Gm26642, Arg1, Cxcl3, Gm590, Sema6b, 5830411N06Rik, Gm37768, Traj11, Gm38319, AC133083.2, Traj22, Gm45803, Gm17606, Klhl35, Gm26835, AC132474.1, Ankrd55, Ccl22, Gm37105 and Il10; or both.
37. Use of the biomarker of any one of embodiments 30-32 to develop or modify a treatment regimen for a subject.
38. A method of improving immune-cell mediated therapeutic treatment by reducing expression and/or function of androgen receptor, essentially as described herein.

### Example 1: Suppressing Androgen Receptor Activity in T Cells Promotes Effective Checkpoint Blockade

Immune checkpoint blockade has revolutionized the field of oncology inducing durable anti-tumor immunity in many solid tumors. In advanced prostate cancer patients, immunotherapy treatments have largely failed (Antonarakis et al., J Clin Oncol 38:395-405, 2020; Beer et al., J Clin Oncol 35:40-47, 2017; Fong et al., In American Society of Clinical Oncology, 2019; Kwon et al., Lancet Oncol 15:700-712, 2014; and Sharma et al., J Clin Oncol. 37(7_suppl): 142-142, 2019). Androgens have pleiotropic functions in prostate cancer; they are potent drivers of cancer cell growth and can suppress T cell function (Kissick et al., Proc Natl Acad Sci USA 111:9887-9892, 2014). In metastatic prostate cancer, intratumor androgen levels can remain high. Androgen deprivation therapy, the backbone of clinical care in prostate cancer, is classically administered to inhibit tumor cell growth. It is postulated that this therapy also impacts tumor associated T cells. To test this hypothesis, transcriptome profiling was performed of individual leukocytes isolated from metastatic prostate cancer lesions resistant to androgen deprivation therapy (ADT) and androgen receptor (AR) antagonism and prior to treatment with a PD-1 inhibitor. This example reveals that inhibition of AR activity in CD8 T cells prevented T cell exhaustion and improved responsiveness to PD-1 targeted therapy via increased IFNγ expression. Notably, AR directly bound to Ifng and eviction of this transcription factor with a small molecule significantly increased cytokine production in CD8 T cells. Furthermore, genomic deletion of Ar prevented chronically stimulated CD8 T cells from losing their ability to make IFNγ upon restimulation. Finally, it is demonstrated that AR blockade sensitizes multiple tumor mouse models to effective checkpoint blockade by directly enhancing CD8 T cell function. Together, the findings establish T cell specific AR activity as one mechanism of immunotherapy resistance and show that targeting AR can improve immunotherapy outcomes.

Immune checkpoint blockade has revolutionized cancer care in many solid tumors. In metastatic castration resistant prostate cancer (mCRPC), the use of checkpoint blockade has largely failed to prolong overall survival (Antonarakis et al., J Clin Oncol 38:395-405, 2020; Fong et al., In American Society of Clinical Oncology, 2019; Kwon et al., Lancet Oncol 15:700-712, 2014; Sharma et al., J Clin Oncol. 37(7_suppl): 142-142, 2019; and Small et al., Clin Cancer Res 13:1810-1815, 2007). Sex-dependent differences in response to immunotherapy are reported (Conforti et al., Lancet Oncol 19:737-746, 2018). Despite evidence that androgens suppress T cell function and IFNγ production (Kissick et al., Proc Natl Acad Sci USA 111:9887-9892, 2014), it is unclear if sex hormones can directly impact the effectiveness of T cell targeted cancer immunotherapies. Importantly, T cells express sex hormone receptors (Benten et al., FASEB J 13:123-133, 1999; Sader et al., Clin Endocrinol (Oxf) 62:56-63, 2005) including AR. In cancer immunotherapy trials, clinical response has been associated with a strong intratumor IFNγ signature prior to immunotherapy initiation (Ayers et al., J Clin Invest 127:2930-2940, 2017; Prat et al., Cancer Res 77:3540-3550, 2017; and Riaz et al., Cell 171:934-949.e916, 2017). Therefore, it is postulated that one mechanism of immunotherapy resistance in prostate cancer patients could be through androgen mediated repression of IFNγ.

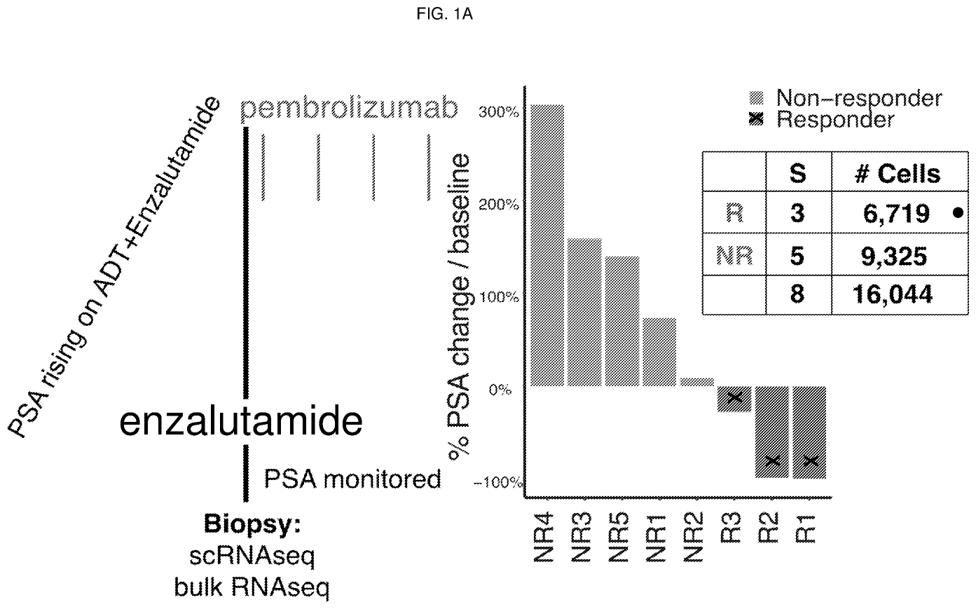

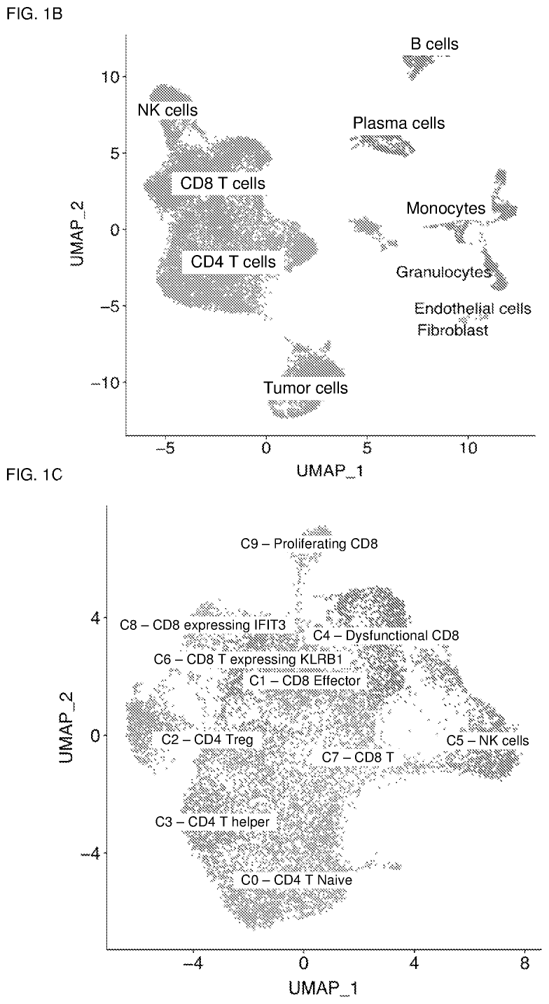

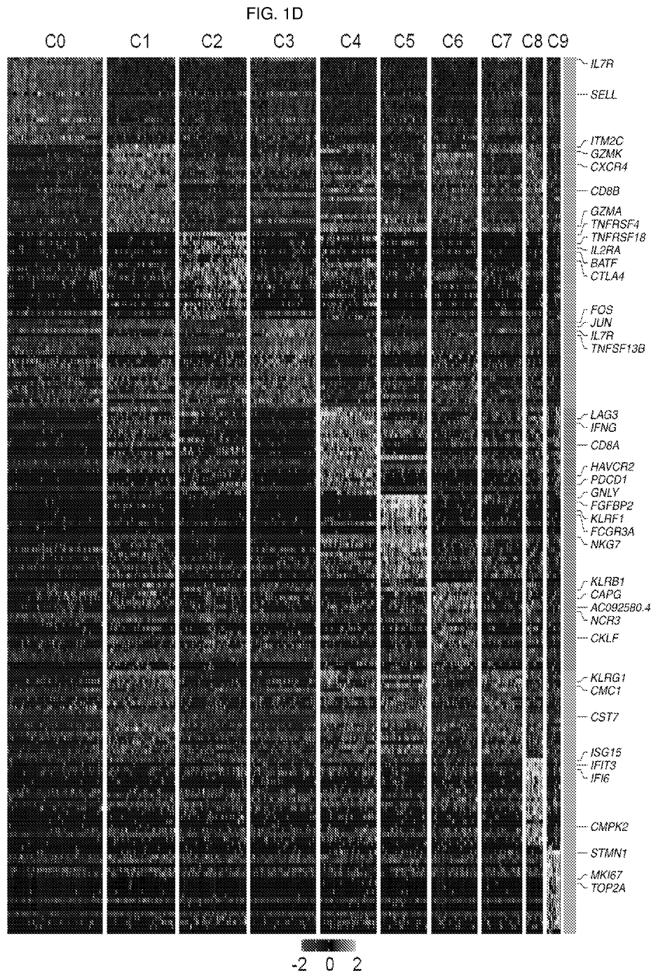

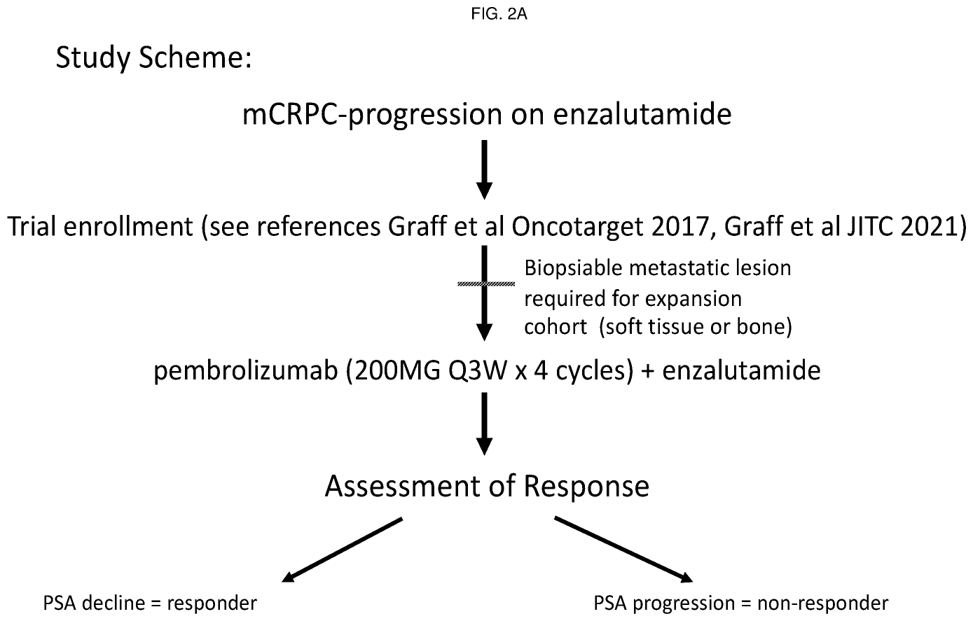

The mainstays of treatment for metastatic prostate cancer are therapies that interrupt androgen signaling. These include medical or surgical castration (known as androgen deprivation therapy and orchiectomy, respectively), inhibition of CYP17 which perturbs production of androgen outside of the testicles, and AR antagonists that block interactions between androgens and AR or dislodge AR from DNA. While therapy is intended to target tumor cells, AR is also widely expressed on multiple tumor-associated leukocyte populations. Notably, AR inhibition with PD-1 blockade (NCTO2312557) (FIGS. 1A and 2A) resulted in a response rate of 18%, challenging the paradigm that immunotherapy would not work in advanced prostate cancer patients (Graff et al., Oncotarget7(33):52810-52817, 2016; and Graff et al., J Immunother Cancer 8(2), 2020) This clinical response raised the possibility that AR inhibition had directly or indirectly enabled T cells to respond to PD-1 inhibition.

To dissect the anti-tumor immune response, a high dimensional single cell RNA sequencing was implemented of freshly isolated tumor-associated leukocytes from metastatic lesions of men enrolled in an expansion cohort prior to treatment with pembrolizumab (FIGS. 1A and 2A). The therapeutic response was compared with transcriptionally defined T cell states prior to anti-PD-1 treatment and focused the analysis on two transcriptionally unique CD8 T cell states associated with response or resistance to PD-1 blockade. Using castration resistant prostate cancer and androgen-independent sarcoma mouse models, the clinical benefits were recapitulated of AR blockade in combination with immune checkpoint therapy. Furthermore, the functional significance of AR signaling in CD8 T cells was demonstrated and revealed that AR can directly regulate T cell cytolytic function.

At least some of the material in this Example was published online on Mar. 23, 2022, as Guan et al. (Nature 606:791-792, 2022; doi.org/10.1038/s41586-022-04522-6), and the extended Data published therewith.

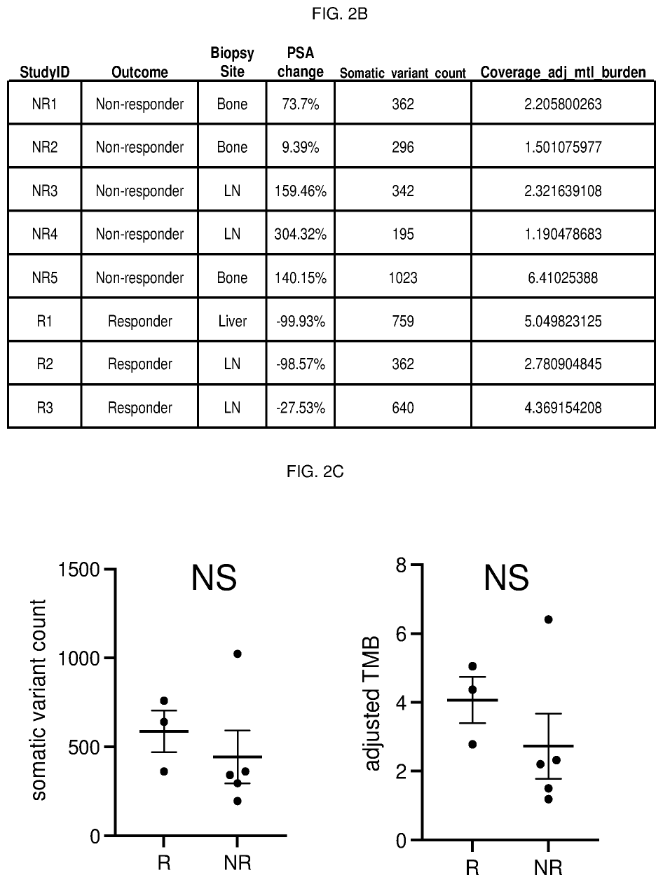

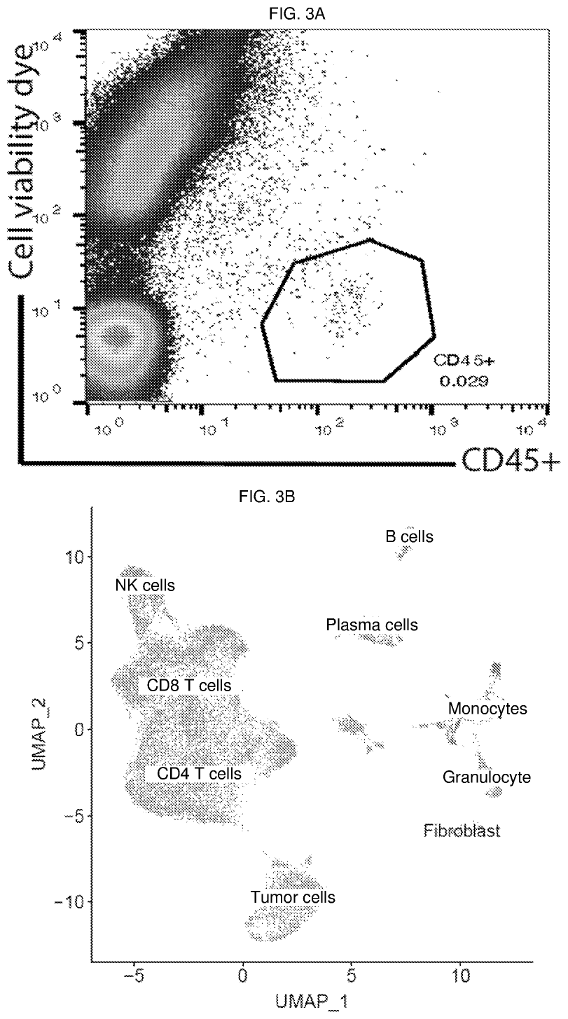

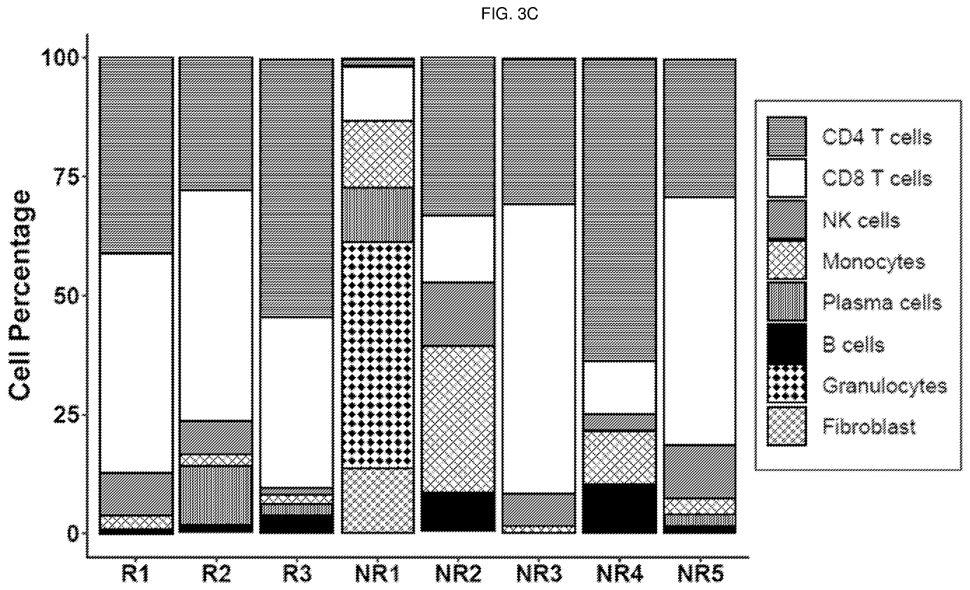

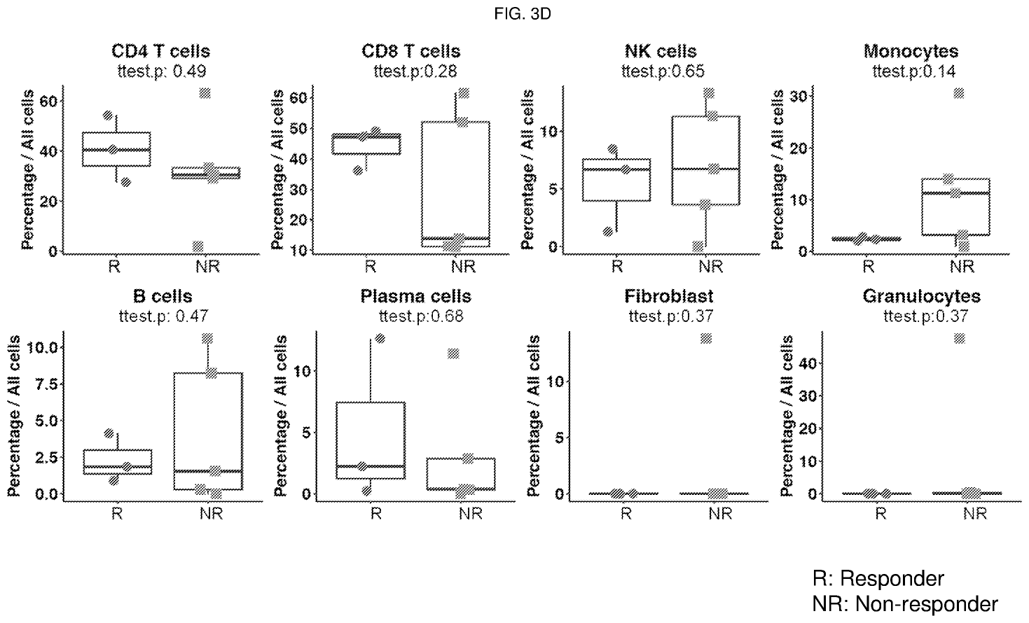

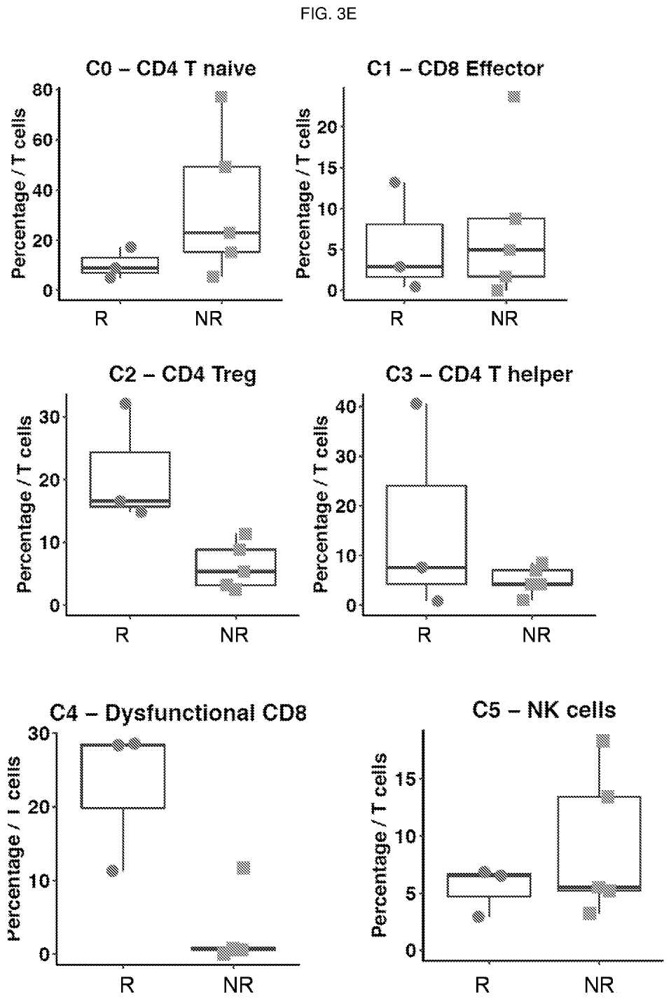

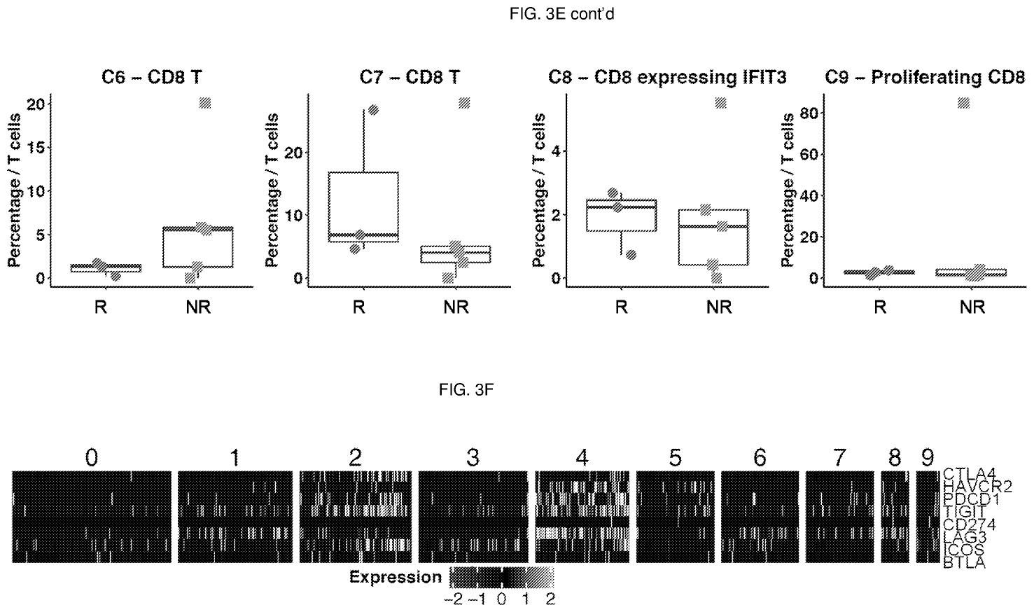

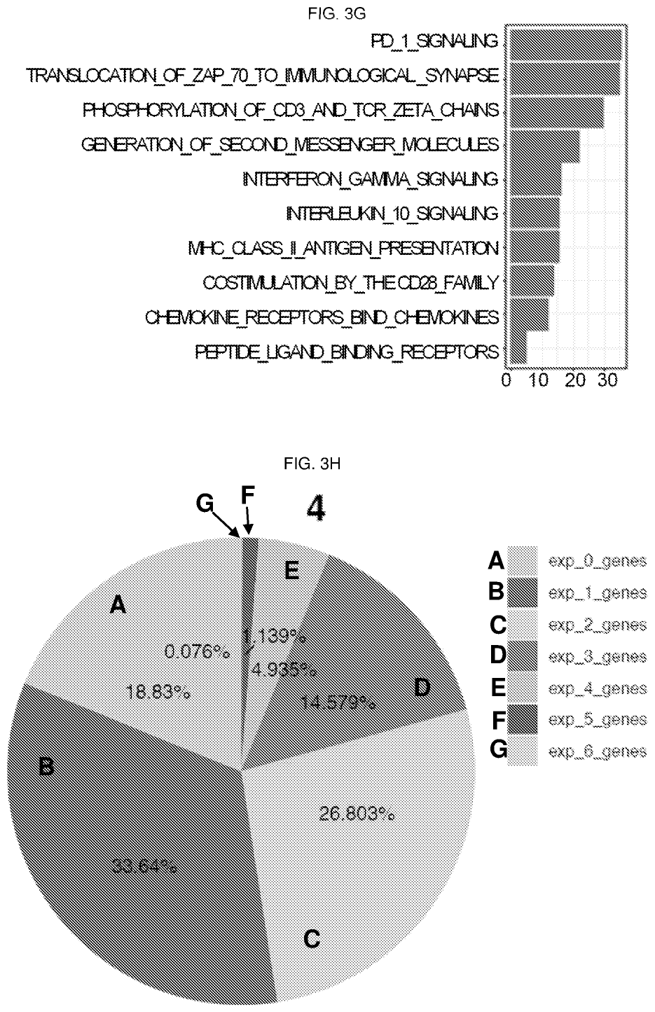

Single-cell immune landscape of tumors from patients with mCRPC prior to checkpoint therapy. To gain insight on androgen regulation of response and/or resistance in this clinical cohort, single cell RNA sequencing (scRNA-seq) was performed on cells isolated from eight individual metastatic tumor lesions from men who had biochemical or radiographic progression on enzalutamide prior to treatment with pembrolizumab. This included three responders and five non-responders, with response defined by a PSA decline of >25% upon immune checkpoint blockade (FIGS. 1A and 2B). Biopsies were obtained and tumor infiltrating leukocytes (TIL) sorted (FIG. 3A). Cells from all patients were merged and the data was clustered into tumor cells and major lymphoid and myeloid immune cell subsets (FIGS. 1B, 3B, 3C; Table 6, which shows the percentage of cell types in each sample calculated out of leucocytes). Although responders and non-responders showed widespread heterogeneity (FIGS. 3B and 3C), there was no significant difference in CD4 T, CD8 T, NK, B, myeloid cell proportions or tumor mutational burden to explain the overall response to checkpoint therapy (FIGS. 2C and 3D). Consistent with other reports, all responders exhibited an abundance of CD8 T cells of total leukocytes sequenced (50% out of all immune infiltrates, FIG. 3D) (Chen et al., Cancer Discov 6:827-837, 2016; and Tumeh et al., Nature 515:568-571, 2014). Notably, two of the five non-responders showed an abundance of CD8 T cells (FIG. 3D), indicating that tumor-associated CD8 T cell abundance was insufficient to stratify responders from non-responders. The wide distribution of B cells and monocytes in non-responders compared to responders was also noted; an observation under further investigation.

TABLE 6

| | R1 | R2 | R3 | NR1 | NR2 | NR3 | NR4 | NR5 |
|---|---|---|---|---|---|---|---|---|
| CD4 T cells | 40.5 | 27.52 | 54.31 | 2.1 | 33.3 | 30.45 | 63.25 | 29.11 |
| CD8 T cells | 47.13 | 49.01 | 36.08 | 13.09 | 13.79 | 61.54 | 11.26 | 52 |
| NK cells | 8.45 | 6.69 | 1.26 | 0 | 13.34 | 6.73 | 3.64 | 11.33 |
| Monocytes | 2.84 | 2.31 | 2 | 16.16 | 30.58 | 0.96 | 11.26 | 3.17 |
| B cells | 0.88 | 1.84 | 4.12 | 0.32 | 8.26 | 0 | 10.6 | 1.55 |
| Plasma cells | 0.19 | 12.63 | 2.23 | 13.25 | 0.36 | 0.32 | 0 | 2.86 |
| Granulocytes | 0 | 0 | 0 | 55.09 | 0.36 | 0 | 0 | 0 |

Unsupervised clustering of all T/NK lymphocytes yielded three CD4 clusters, six CD8 clusters, and one NK/NKT cluster (FIGS. 1C and 1D). Among the different subsets of T/NK cells, responder lesions were enriched for CD8 T cells expressing genes associated with antigen encounter and dysfunction (C4 cluster, p=0.057 two-tailed Student's t-test; FIG. 3E) as noted by higher transcripts of PDCD1, LAG3, HAVCR2, TIGIT, and IFNG (FIGS. 1D and 3F, Supplementary Table 2 in Appendix B of U.S. Provisional Application No. 63/319,712). Pathway analysis revealed enrichment of TCR signaling and PD-1 signaling in C4 cluster (FIG. 3G), data consistent with TCR signaling inducing transcripts shared by activated and exhausted CD8 T cells (FIG. 3H) (Wherry et al., Immunity 27:670-684, 2007). Together, these data revealed the presence of multiple lymphocyte subsets in mCRPC lesions. For the purposes of understanding if androgen signaling could limit immunotherapy effectiveness, the analysis focused on CD8 T cells given their critical role in PD-1 targeted immunotherapy.

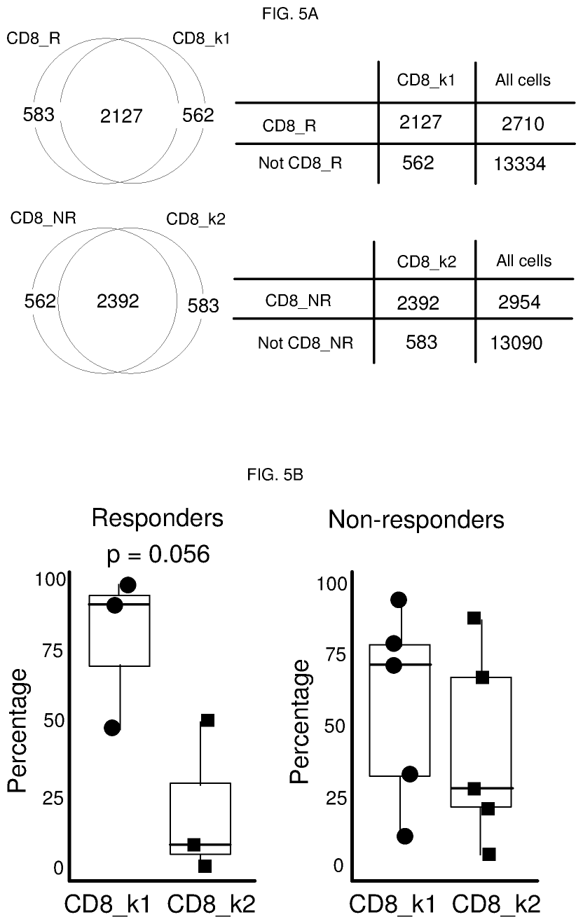

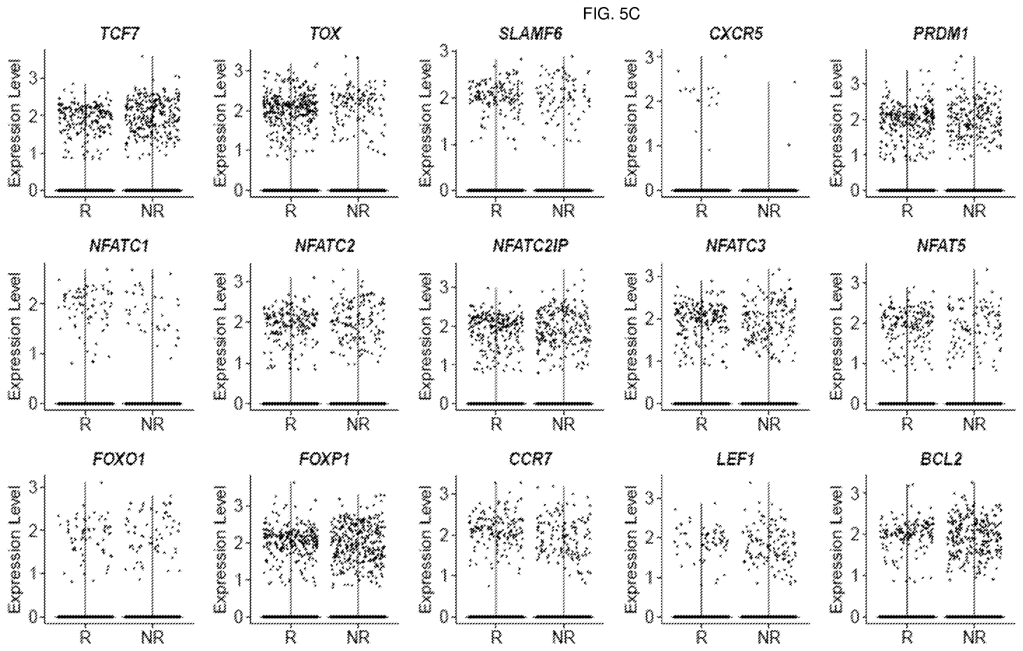

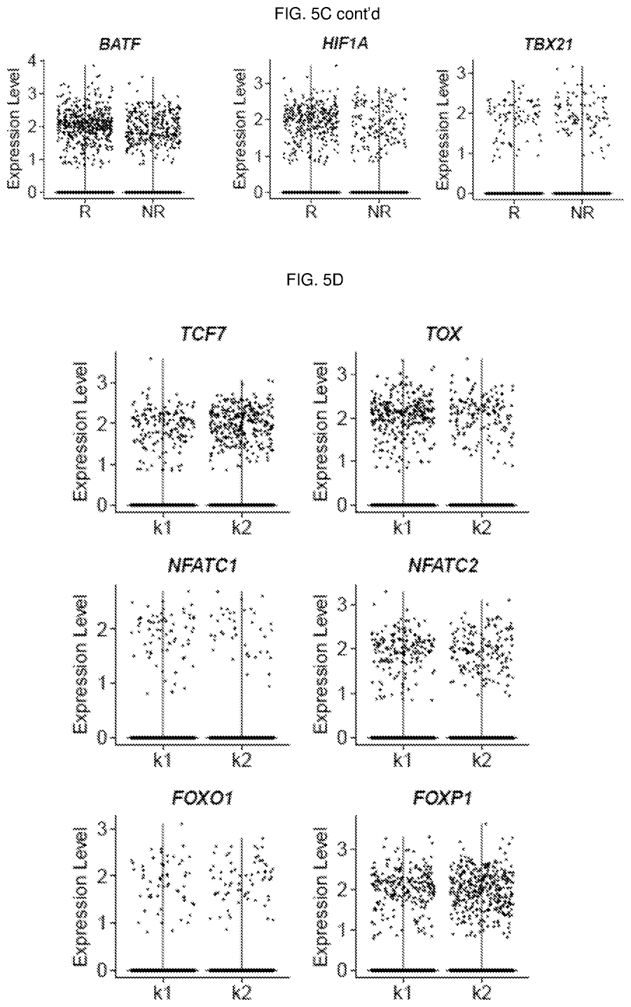

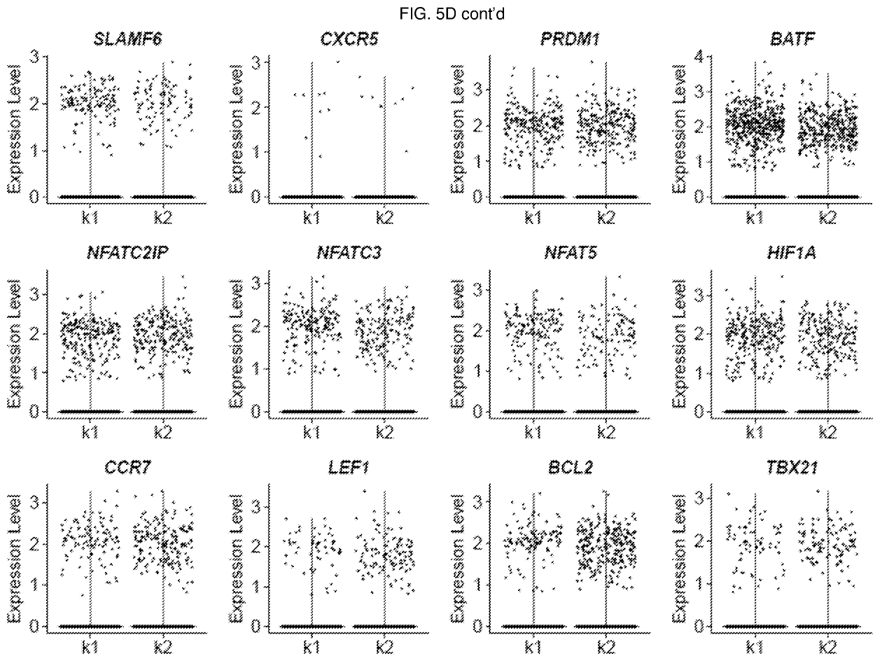

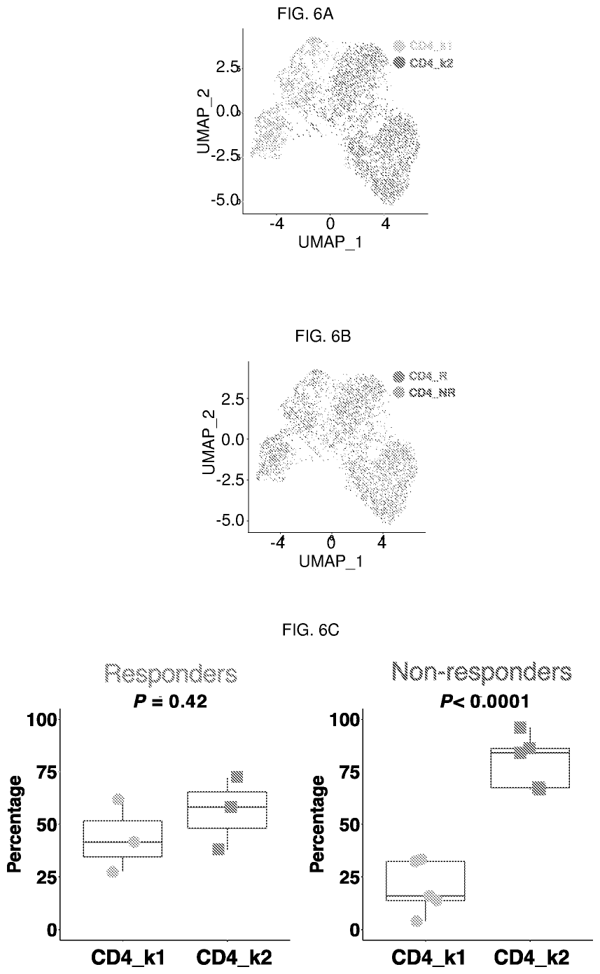

Unbiased nomination of transcriptome signatures in CD8 T cells that are associated with checkpoint therapy responses. Given that two non-responder lesions were abundantly infiltrated with CD8 T cells yet failed to respond, the possibility was considered that there was a T cell intrinsic cell state associated with response, similar to that reported in non-prostate solid tumor studies (Ayers et al., J Clin Invest 127:2930-2940, 2017; Prat et al., Cancer Res 77:3540-3550, 2017; and Riaz et al., Cell 171:934-949.e916, 2017). Considering the two therapeutic outcomes to PD-1 blockade, response or non-response, unsupervised clustering was performed of CD8 T cells from all eight patients to define two cell states based on transcriptome (FIG. 4A). CD8_k1 cells (k-means cluster 1 CD8 T cells) had higher expression of inhibitory genes (HAVCR2, LAG3, PDCD1, TIGIT) and genes associated with cytotoxicity (GZMB, TNFRSF9), and MHC II class (HLA-DRB1, HLA-DRA, HLA-DQA1, and HLA-DQB1), and CD8_k2 (k-means cluster 2) cells had increased expression of heat-shock genes (HSP90AA1, HSPA1A, and HSPA1B) (FIG. 4B, Supplementary Table 3 in Appendix B of U.S. Provisional Application No. 63/319,712). CD8_k2 cells also exhibited enhanced expression of BTG1 and BTG2 (FIG. 4B), recently shown to be involved in the maintenance of T cell quiescence (Hwang et al., Science 367:1255-1260, 2020). CD8 T cells were also characterized based on clinical response to anti-PD-1 blockade. Remarkably, the unsupervised k-means clusters, CD8_k1 and CD8_k2, very closely overlapped with CD8 T cells from responders (CD8_R) and non-responders (CD8_NR) at both cell population and gene expression profiles (FIGS. 4C, 4D, 5A, 5C, and 5D, Supplementary Table 3 in Appendix B of U.S. Provisional Application No. 63/319,712). Furthermore, previously reported genes associated with a CD8 T cell state associated with response to PD-1 blockade (i.e. TOX, TCF7, FOXP1, BCL2) were minimally expressed in CD8_k1 and CD8_R (FIGS. 5C and 5D). While both CD8 T cell states existed in responders and non-responders, CD8_k1 cells were enriched (albeit not statistically significantly) in responder biopsies (FIG. 5B). Importantly, similar to some non-responder lesions containing an abundance of CD8 T cells (FIGS. 3B and 3D), it was also observed that some contained CD8_k1 cells but failed to respond to PD-1 targeted therapy (FIG. 5B), suggesting a novel cell intrinsic mechanism could restrain T cell function. Notably, some non-responder lesions were enriched for monocytic cells (FIGS. 3B and 3D) compared to responders. In contrast to defined CD8 cell states associated with response, CD4 T cells did not exhibit clear states that were associated with response (FIGS. 6A-6C).

From these distinct CD8 T cell states the differentially expressed genes were computed between CD8 T cells from responders and non-responders (FIG. 4D, Supplementary Table 3 in Appendix B of U.S. Provisional Application No. 63/319,712) and performed master regulator (MR) analysis. This algorithm infers differentially activated transcription factors in a defined gene signature (i.e. CD8_R vs CD8_NR) based on the enrichment of each transcription factors gene targets (Alvarez et al., Nat Genet 48:838-847, 2016; and Lefebvre et al., Mol Syst Biol 6:377, 2010). This analysis predicted the deactivation of multiple transcription factors including the androgen receptor (AR) in CD8 T cells from responders (FIG. 4E).

Next, the biological processes enriched in CD8 T cells were queried from responders vs non-responders. This analysis associated clinical response with high activation of pathways in CD8 T cells including TCR signaling, co-inhibitory signaling, PD-1 signaling, and IFNγ signaling (FIG. 4F). Interestingly, the increase in these pathways corresponded with the deactivation of the Nr4a1 pathway (FIG. 4E); a transcription factor pathway reported to limit the function of tumor-specific CD8 T cells and anti-PD-1 targeted immunotherapy (Chen et al., Nature 567:530-534, 2019). Resistance to PD-1 blockade was associated with an increase in the HSP90 steroid hormone receptor (FIG. 4F), a chaperone protein that can facilitate AR function. Together, these data revealed distinct CD8 T cell states associated with response and resistance to PD-1 blockade and raised the possibility that AR downregulation in CD8 T cells was correlated with improved cell function. To corroborate this hypothesis in an independent dataset, a single cell gene signature was derived from differentially expressed genes between CD8_k1 and CD8_k2, or between CD8_R and CD8_NR and applied this to bulk RNA sequencing data derived from metastatic biopsies from patients from whom fresh tissue could not be obtained for analysis. This approach revealed a negative correlation between single cell signatures and AR signaling based on either the CD8_k1 (FIG. 4G) or CD8_R cell states (FIG. 4H), which further suggested that favorable T cell signatures were associated with low AR signaling, a state more likely to respond to PD-1 blockade.

Considering that the data suggested AR signaling in CD8 T cells negatively correlated with cell functionality and response to immunotherapy, it was evaluated whether there was evidence of lymphocytes expressing AR within prostate cancer patients. In fact, in routine AR staining of treatment naïve primary prostate cancer patients, AR positive TIL was observed in multiple patients (FIG. 4I). Furthermore, AR mRNA was readily detectable in human CD8 T cells by qPCR (FIG. 4J), and markedly increased after TCR stimulation (FIG. 4J).

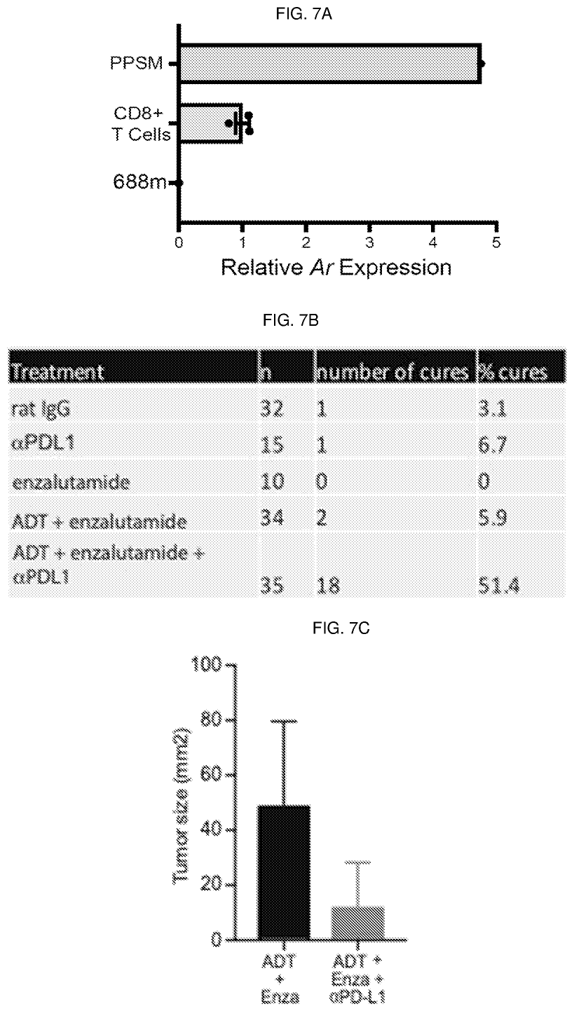

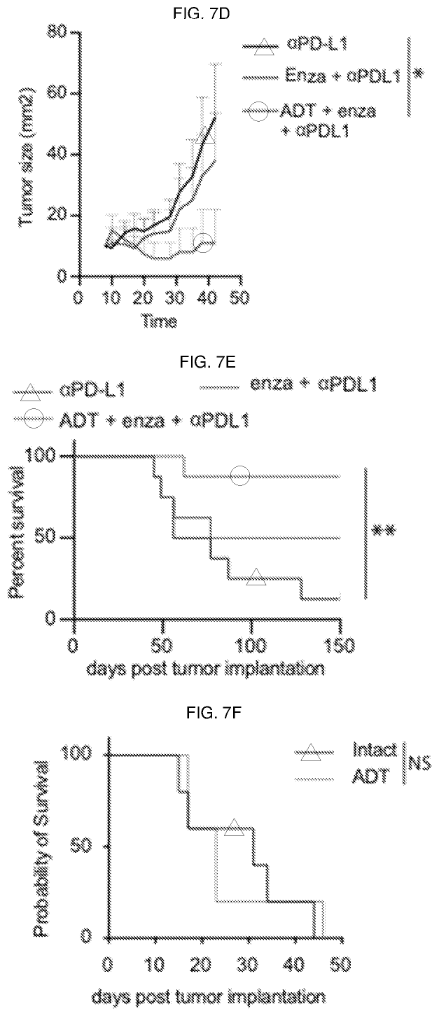

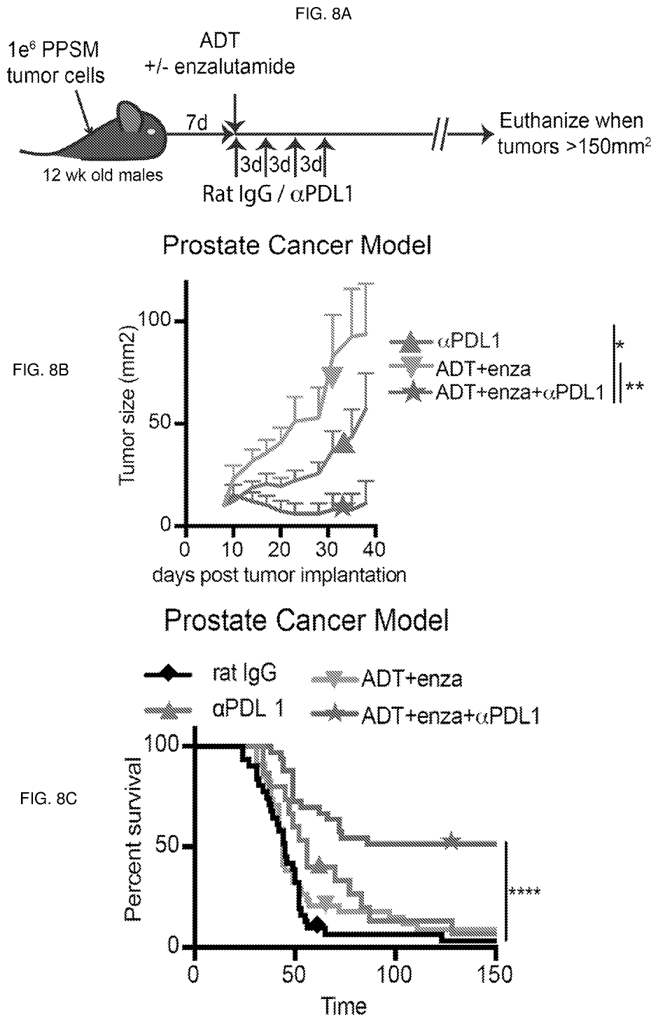

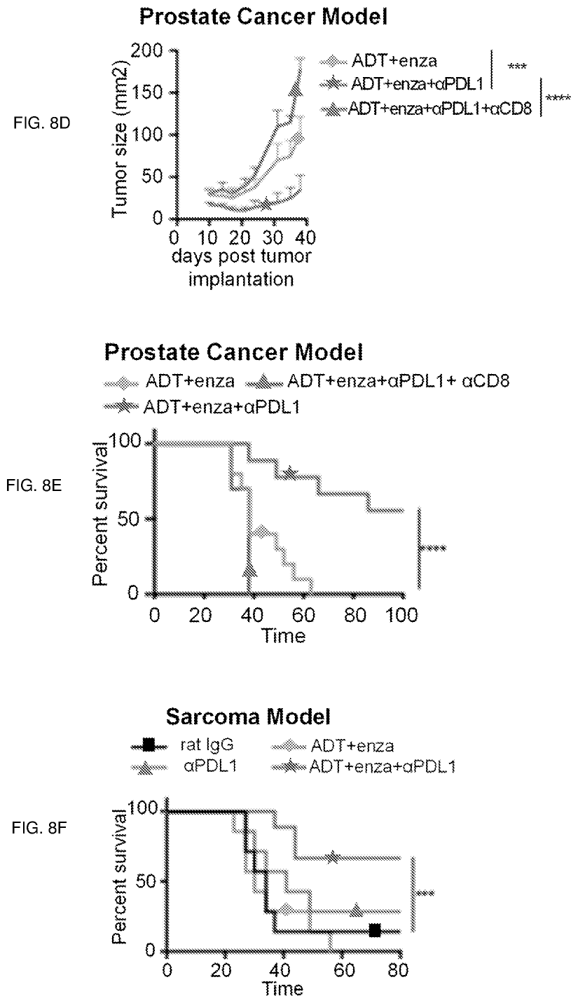

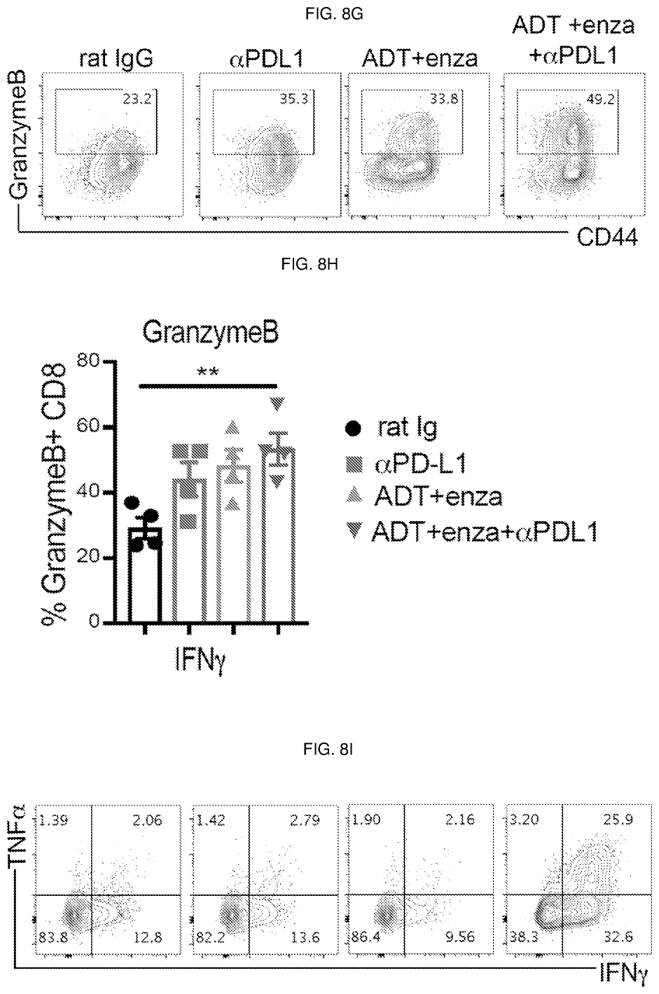

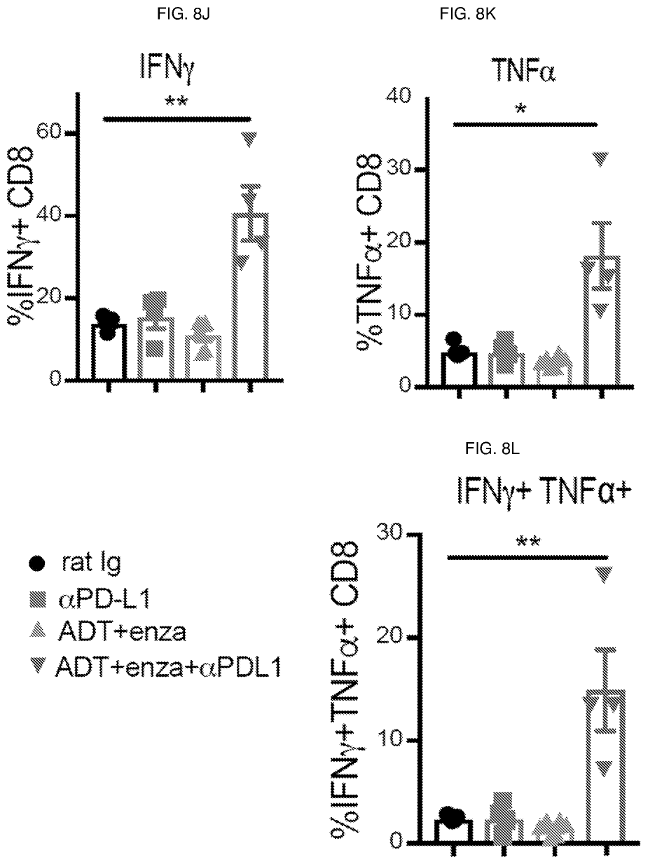

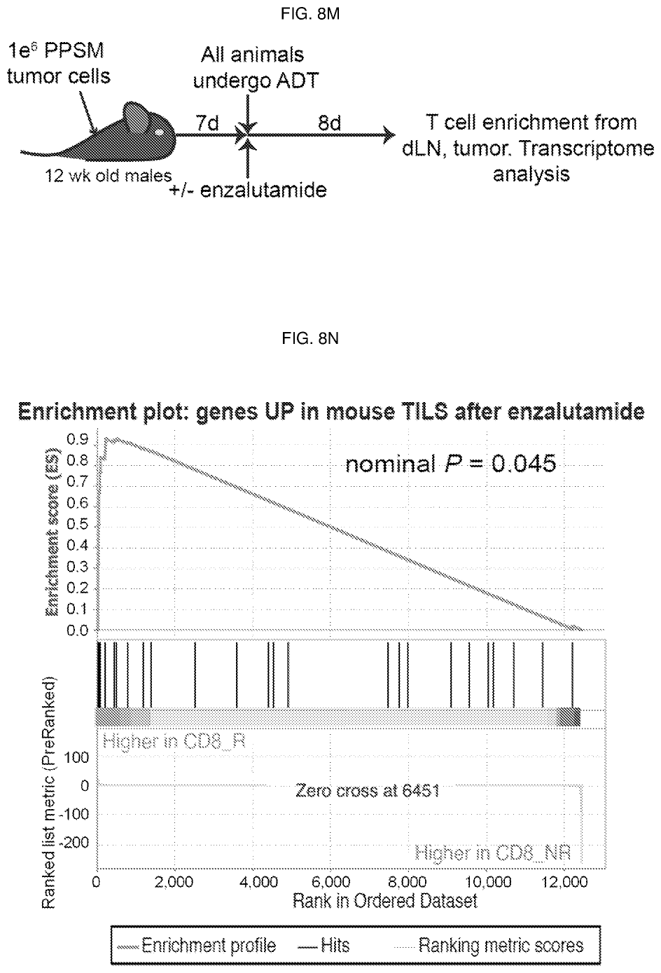

Dual inhibition of AR and PD-1/PD-L1 reduces tumor growth and improves survival/Low AR activity in CD8 T cells appeared to contribute to effective PD-1/PD-L1 blockade in prostate cancer patients. Therefore, to determine if AR perturbation with enzalutamide would enable effective PD-1 axis targeted therapy in a mouse tumor model, mouse CD8 T cells were confirmed to express Ar (FIG. 7A) and then implanted male mice with an ADT and anti-PD-1 resistant tumor (PB-Cre⁺; Pten^{L/L}; p53^{L/L}; Smad4^{L/L}, referred to as PPSM). Following tumor initiation, animals underwent orchiectomy (referred to as androgen deprivation therapy, ADT), and were treated with enzalutamide and/or anti-PD-L1 (FIG. 8A). While monotherapy with either PD-L1 blockade or ADT plus enzalutamide had minimal impact on tumor growth (FIG. 8B), significant tumor regression was observed (FIG. 8B) and increased overall survival in the ADT+enzalutamide+anti-PD-L1 group (FIGS. 8C and 7B). Similar synergy was observed when PPSM tumors were implanted orthotopically (FIG. 7C). PD-L1 blockade, together with enzalutamide treatment in the absence of ADT was not as effective as the combination of ADT+AR inhibition+PD-L1 blockade (FIG. 7D), suggesting that while ADT dramatically reduces testosterone levels, the functional perturbation of AR with the small molecule enzalutamide, was critical for optimal effect. This is consistent with a previous report that suggested enzalutamide in the absence of ADT is immunologically inert (Ardiani et al., Clin Cancer Res 19:6205-6218, 2013). Of note, ADT alone had no effect on tumor growth (FIG. 7F).

Our clinical data suggested that low AR activity in CD8 T cells was associated with therapeutic benefit to PD-1 blockade. Given the critical importance of CD8 T cells in effective PD-1 targeted immunotherapy (Tumeh et al., Nature 515:568-571, 2014), CD8 T cells were depleted from tumor bearing animals and observed a loss of tumor control in the presence of the combination therapy (FIGS. 8D and 8E). Last, while ADT+AR inhibition is typically intended to target the cancer cells, the data suggested a T cell intrinsic role for AR where reduced T cell AR activity improved T cell function. Given this observation, it is reasoned that ADT with AR inhibition might sensitize other immunotherapy refractory tumors to effective checkpoint blockade. It has previously been shown that MCA-205 sarcoma tumor bearing animals are refractory to single agent PD-L1 blockade (Polesso et al., Cancer Immunol Res 7:269-281, 2019). Therefore, male MCA205 tumor bearing mice were treated with mono or combination therapy and it was observed that ADT with enzalutamide also sensitized these animals to effective anti-PD-L1 therapy (FIG. 8F).

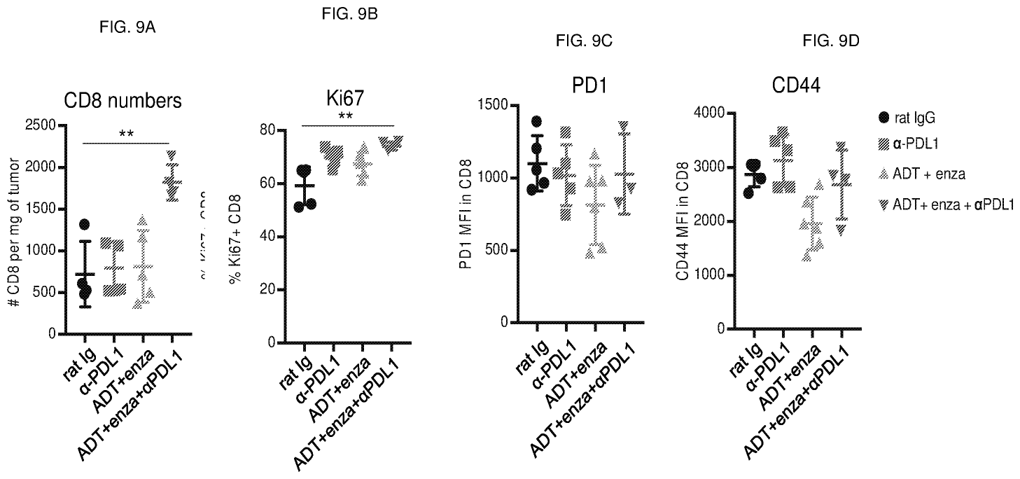

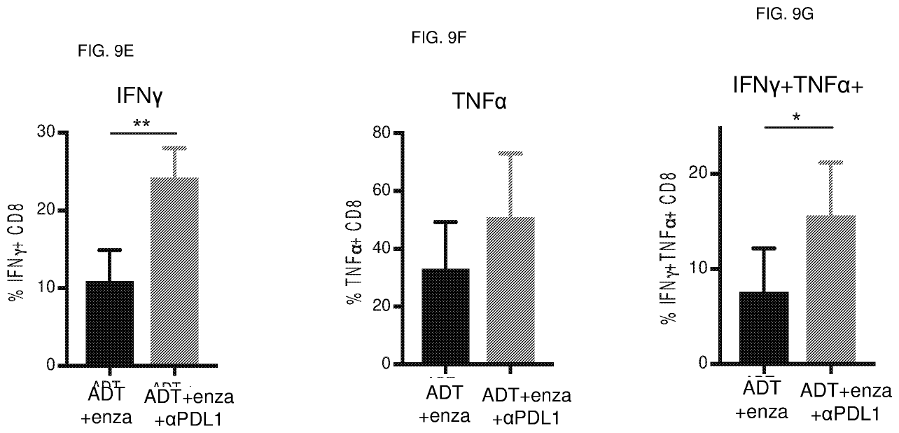

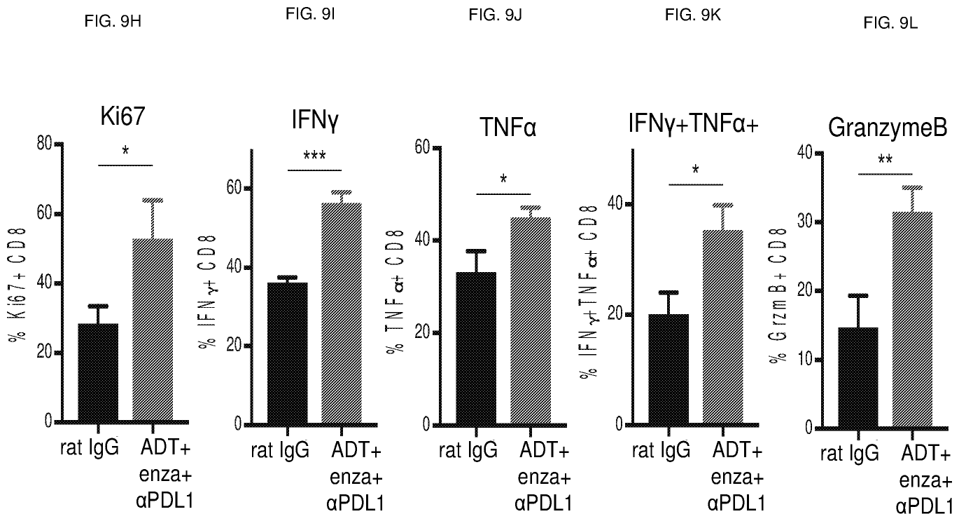

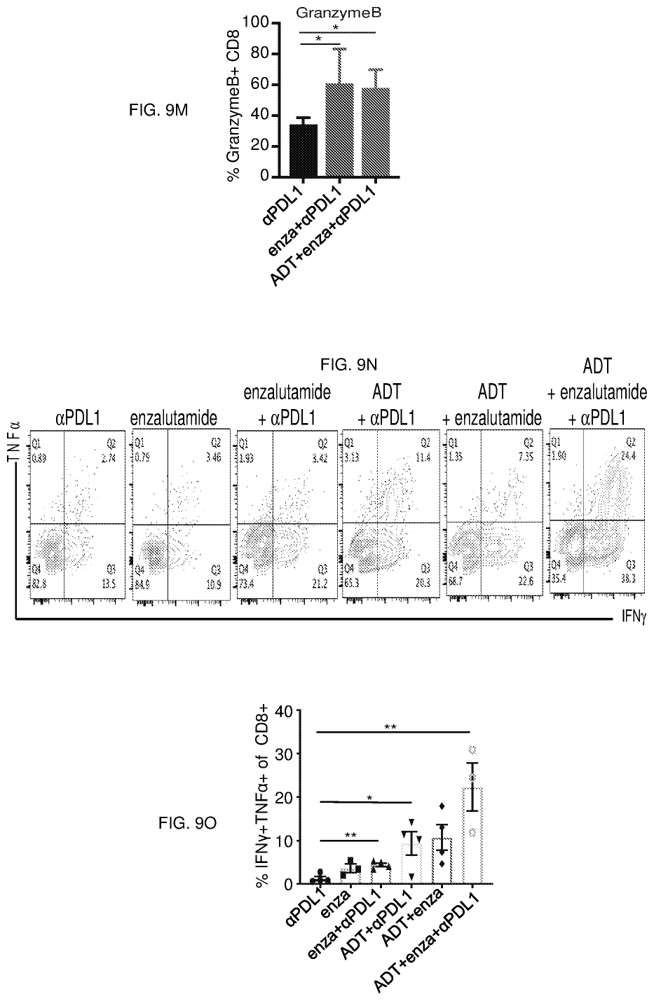

Functional capacity of CD8 T cells is improved with AR inhibition. Based on the pathway analysis performed on CD8_R T cells (FIG. 4F) demonstrating an increase in IFNG pathway activity, the therapeutic relevance was explored of AR inhibition on modulating the expression of Ifng and Gzmb in tumor-infiltrating CD8 T cells in PPSM tumor bearing mice. The total number of CD8 T cells in the tumor was not increased with monotherapy but was significantly increased with ADT+enzalutamide+anti-PD-L1 treatment (FIG. 9A), possibly reflecting an increase in proliferation of these cells (FIG. 9B). The striking difference between groups was the ability of tumor infiltrating CD8 T cells to produce effector cytokines. Granzyme B production was increased with monotherapy and significantly higher compared to control in combination therapy treated animals (FIGS. 8G and 8H). Furthermore, IFNγ and TNFα production was significantly increased in T cells from animals treated with ADT+AR inhibition in combination with anti-PD-L1 blockade (FIGS. 8I-8L). Critically, enzalutamide or anti-PD-L1 alone was insufficient to improve the polyfunctionality of tumor-associated CD8 T cells (FIGS. 8I-8L), suggesting that reduced AR signaling could establish a CD8 T cell state permissive to T cell re-invigoration through PD-1/PD-L1 blockade. Consistent with this idea, ADT+enzalutamide slightly decreased the overall protein expression of PD-1 and CD44 (FIGS. 9C and 9D), a phenotype associated with increased effector function (Blackburn et al., Proc Natl Acad Sci USA 105:15016-15021, 2008; Kansy et al., Cancer Res 77:6353-6364, 2017). Similar increases in cytokine production were observed in orthotopic PPSM tumors (FIGS. 9E-9G) or via ADT with degarelix instead of orchiectomy (FIGS. 9H-9L). The protective benefit of AR inhibition was also evaluated with anti-PDL1 blockade in the absence of ADT and observed a partial increase in T cell effector function (FIGS. 9M-9O). Last, a model of T cell adoptive therapy was employed for sarcoma in male and female tumor bearing animals. All males were chemically castrated and later treated or not with enzalutamide (FIG. 10A). In this adoptive T cell therapy model, enzalutamide treatment increased IFNγ production of tumor-antigen specific CD8 T cells in both male and female mice (FIGS. 10B and 10C).

The success of enzalutamide with PD-1 blockade in the clinical setting (Graff et al., Oncotarget7(33):52810-52817, 2016; and Graff et al., J Immunother Cancer 8(2), 2020) and the mouse tumor studies suggested that AR inhibition with enzalutamide enhanced the benefit of ADT alone on T cell function. To investigate this further, T cells isolated from the tumor of orchiectomized+/−enzalutamide treated male mice implanted with PPSM were transcriptionally profiled (FIG. 8M) (Lu et al., Nature 543:728-732, 2017). Using the genes upregulated in T cells after enzalutamide treatment (Supplementary Table 4 in Appendix B of U.S. Provisional Application No. 63/319,712), gene set enrichment analysis (GSEA) was performed with the ranked gene list derived from CD8_R vs CD8_NR (FIG. 4D, Supplementary Table 3 in Appendix B of U.S. Provisional Application No. 63/319,712). The results showed that AR-inhibited genes in mouse TILs were significantly enriched in patient CD8_R T cells (FIG. 8N).

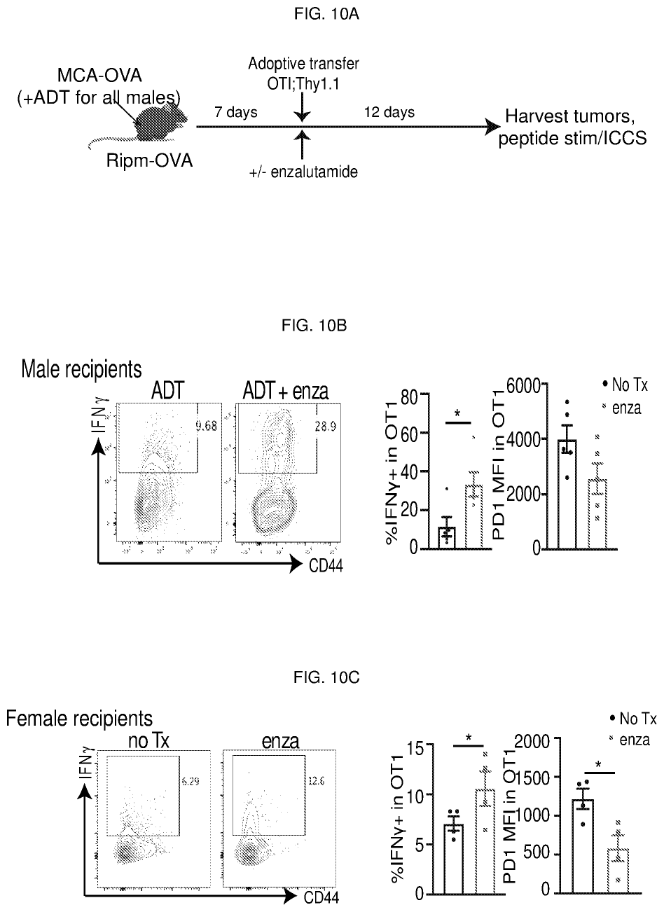

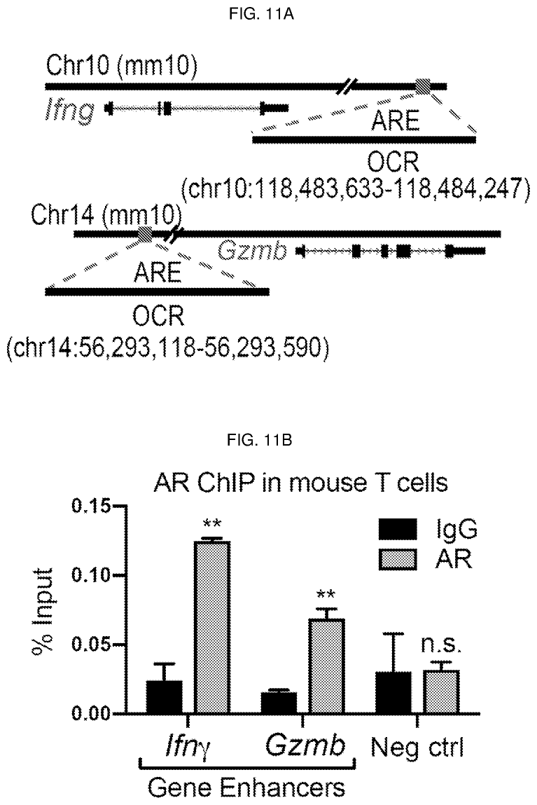

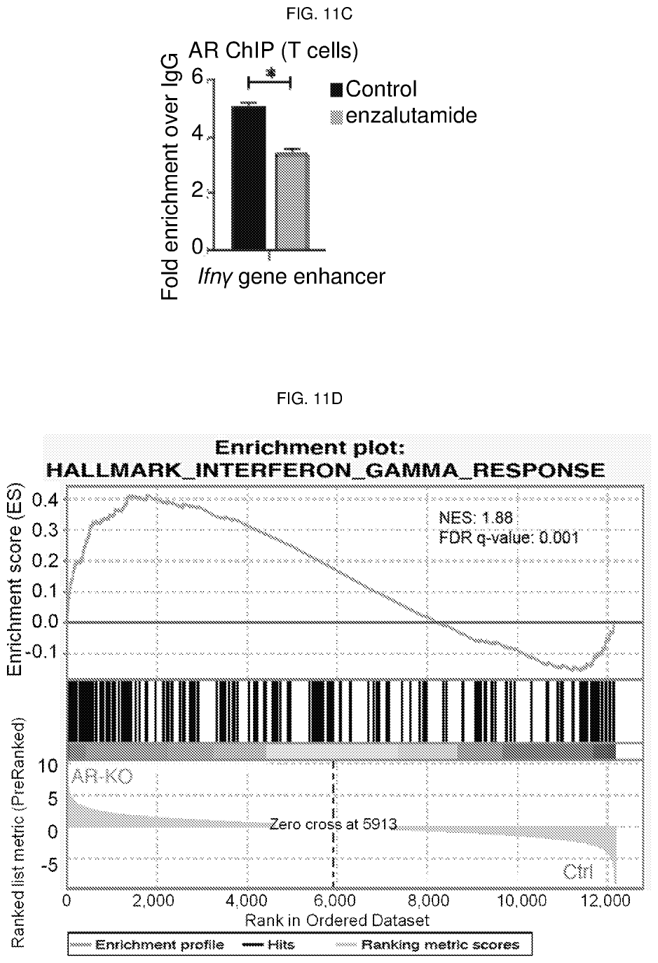

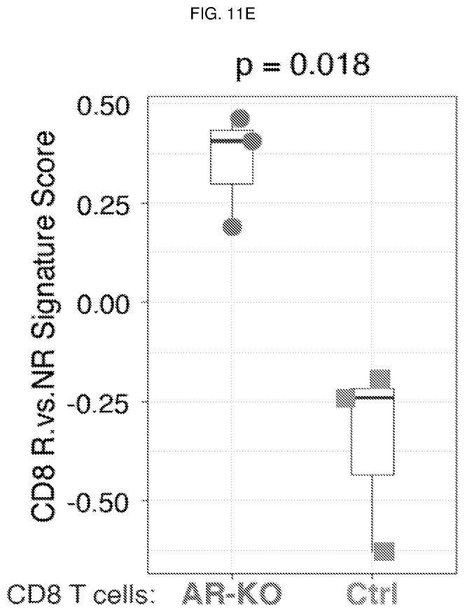

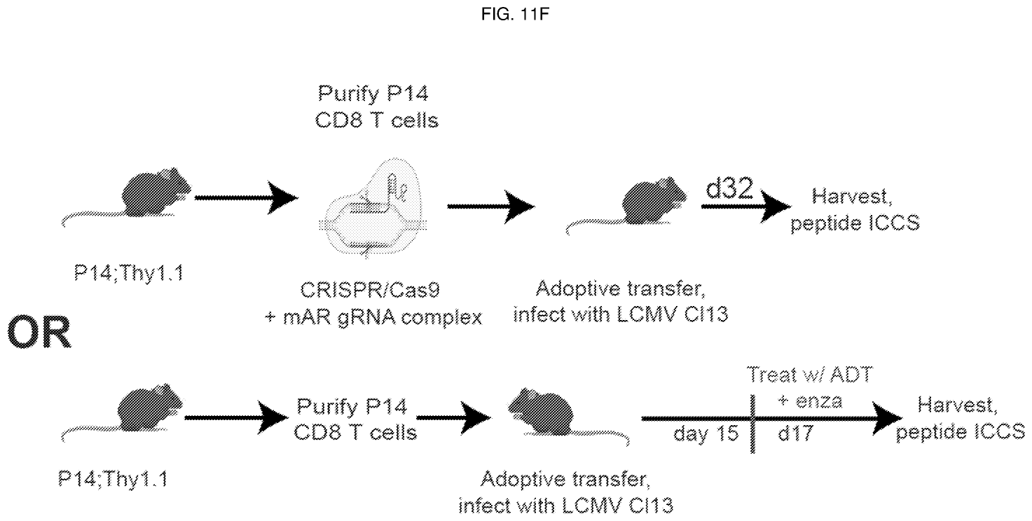

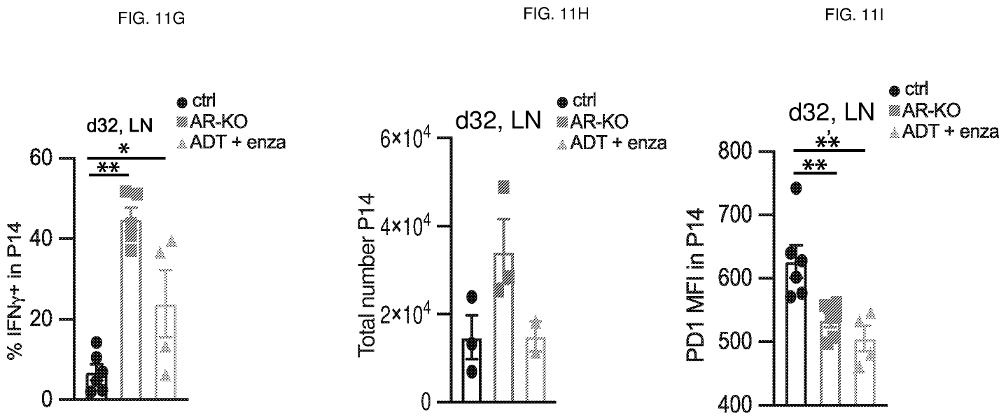

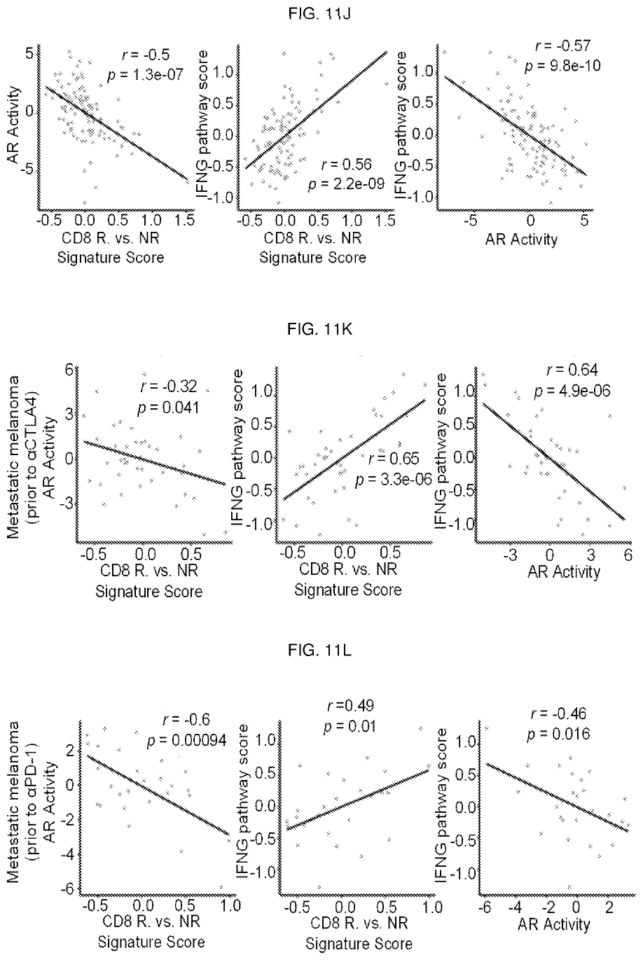

T cell intrinsic AR represses IFNγ activity. A fundamental observation made in the mouse tumor model experiments was the critical role for ADT and AR inhibition in poising CD8 T cells to respond to PD-L1 blockade. One characteristic feature of effector memory CD8 T cells is the presence of open chromatin regions (OCRs) associated with the critical cytolytic effector genes, Ifng and Gzmb (Kersh et al., J Immunol 176:4083-4093; Northrop et al., J Immunol 177:1062-1069, 2006; and Zediak et al., J Immunol 186:2705-2709, 2011) which allow rapid production of cytotoxic cytokines, IFNγ and granzyme B upon TCR ligation. It was hypothesized that AR interacted with the Ifng and Gzmb genes in OCRs associated with functional state. To explore this hypothesis, OCRs were screened in Ifng and Gzmb genes at either the CD8 effector or memory cell state (Pauken et al., Science 354(6316):1160-1165, 2016) for canonical androgen response elements (AREs) using JASPAR database of curated transcription factor binding profiles (Fornes et al., Nucleic Acids Res. 48(D1):D87-92, 2020; doi: 10.1093/nar/gkz1001). Robust AREs were identified in OCRs associated with Ifng (FIG. 11A) in effector memory CD8 T cells (Table 7). AREs were also identified in OCRs associated with Gzmb in addition to the Ifng gene (FIG. 11A).

TABLE 7

| OCRs in | | | | AR binding |
|---|---|---|---|---|
| Ifng | | | | (ChlP- |
| gene | Location (mm10) | T cell type | ARE | qPCR) |
| OCR#1 | chr10: 118482539-118483108 | Effector & Effector- | NO | Negative |
| | | memory | | |
| OCR#2 | chr10: 118483633-118484247 | Effector & Effector- | YES | Positive |
| | | memory | | |
| OCR#3 | chr10: 118404226-118405089 | Effector-memory | NO | NA |
| OCR#4 | chr10: 118399912-118400371 | Effector-memory | NO | NA |
| OCR#5 | chr10: 118455661-118456276 | Effector-memory | YES | Negative |
| OCR#6 | chr10: 118399049-118399742 | Effector-memory | NO | NA |
| OCR#7 | chr10: 118495148-118495823 | Exh_aPD1 | NO | NA |
| OCR#8 | chr10: 118386261-118387216 | Exh_aPD1 | YES | NA |

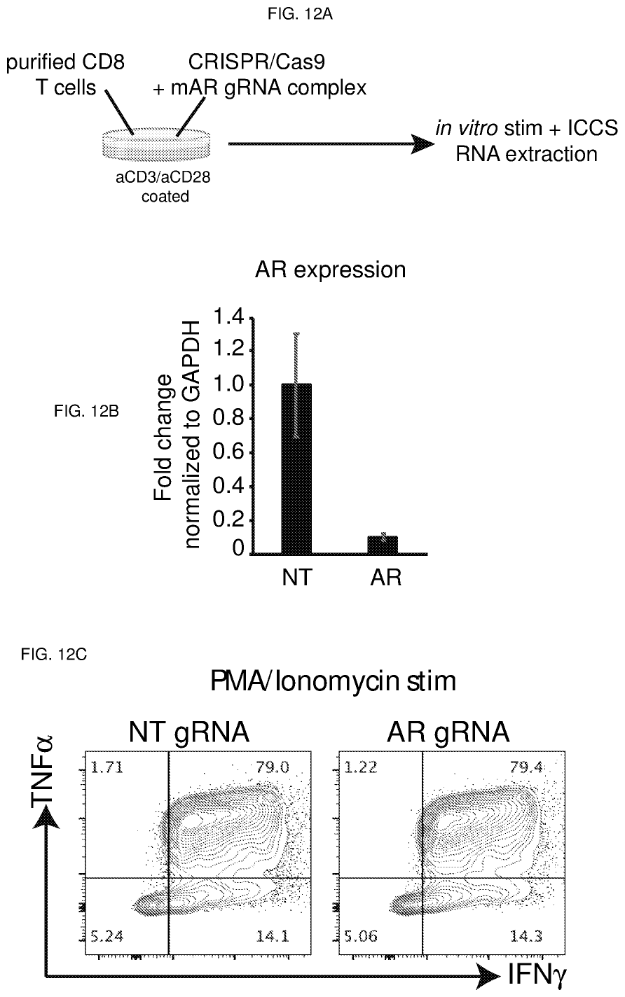

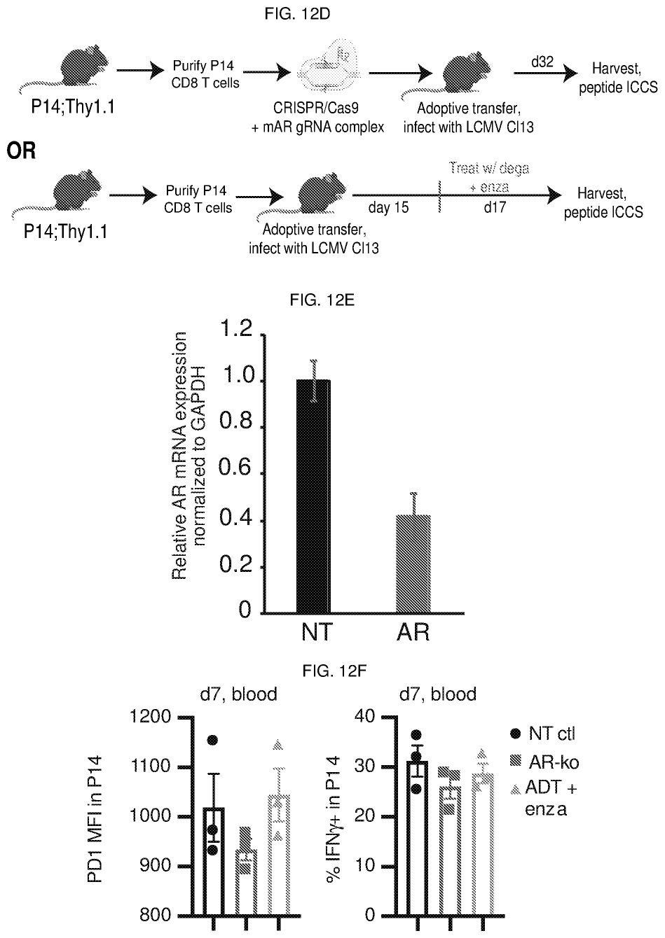

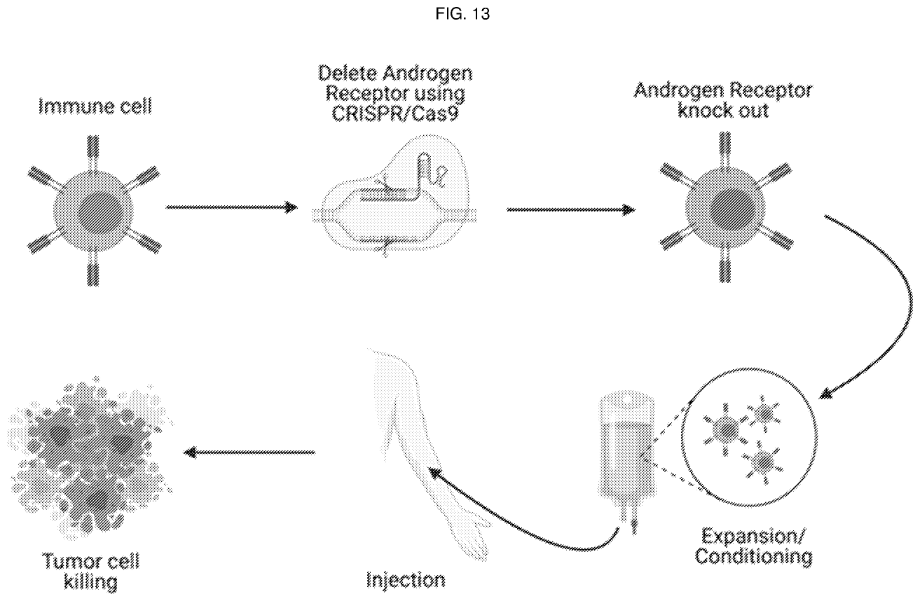

To directly assess if AR could be regulating CD8 T cell function through binding to the OCRs (Table 7), a chromatin immunoprecipitation was performed of AR from activated T cells and measured the enrichment of the chromatin regions using qPCR. AR could bind an OCR in Ifng and Gzmb (FIG. 11B) and the binding was significantly reduced by enzalutamide treatment (FIG. 11C). Taken together, this suggests that AR can directly bind and regulate the expression of Ifng and that enzalutamide perturbs this regulation by dislodging AR from the chromatin. To further investigate this observation, Ar was deleted from primary CD8 T cells (Nussing et al., J Immunol 204:2308-2315, 2020) and stimulated them for 3 days in vitro (FIGS. 12A and 12B) before performing RNA sequencing. GSEA analysis was performed on the differentially expressed genes in Ar deficient or sufficient effector CD8 T cells and revealed a significant enrichment of the hallmark IFNG response in Ar knockout CD8 T cells compared to wildtype controls (FIG. 11D). Moreover, CD8_R signature was significantly enriched in knockout CD8 T cells (FIG. 11E).

Based on these observations, it was hypothesized that loss of AR may protect chronically stimulated CD8 T cells from losing their capacity to make IFNγ (Wherry et al., J Virol 77:4911-4927, 2003). To test this idea, a classic model of T cell exhaustion was used, the lymphocytic choriomeningitis virus clone 13 (LCMV CI13) model (Ahmed et al., J Exp Med 160:521-540, 1984) (FIG. 11F). Strikingly, Ar deficient P14 CD8 T cells retained the capacity to produce IFNγ upon peptide stimulation more than 30 days after infection (FIG. 11G). Likewise, treatment of infected animals with ADT+enzalutamide 2 weeks before harvest partially restored P14 IFNγ production (FIG. 11G). Importantly, there were more antigen specific T cells (FIG. 11H) and PD-1 expression levels were lower when Ar was deleted (FIG. 11I).

Last, the possibility was evaluated that the single cell signature derived from response features in CD8_R could delineate mCRPC patients into AR^{low} IFNγ^{hi} and AR^{hi}IFNγ^{low}, a potential biomarker to identify patients that might benefit from PD-1 blockade. Indeed, it was observed that the CD8_R signature negatively correlated with AR activity (FIG. 11J) and positively correlated with IFNG pathway activity in a larger cohort of mCRPC patients (Quigley et al., Cell 174:758-769. e759, 2018). Moreover, it was postulated that this relationship between the CD8_R signature, IFNG activity, and AR activity could be extended beyond prostate cancer, similar to the therapeutic synergy demonstrated in mouse models of sarcoma and adoptive T cell therapy (FIGS. 8F and 10A-10C). In two independent melanoma datasets from patients prior to treatment with anti-CTLA4 therapy (FIG. 11K) (Van Allen et al., Science 350:207-211, 2015) or anti-PD-1 therapy (FIG. 11I) (Hugo et al., Cell 168:542, 2017), similar negative correlations were observed between AR activity and CD8_R signature and IFNG pathway activity. Taken together, the findings establish that T cell intrinsic AR activity is an unappreciated mechanism of IFNG suppression and immunotherapy resistant, limiting effective checkpoint blockade immunotherapy. It was proposed that in advanced prostate and immunotherapy resistant cancer patients, intratumor androgens may represent a novel mechanism of therapy resistance. Moreover, T cell intrinsic AR activity may serve as a useful biomarker for identifying patients who could achieve clinical benefit with PD-1/PD-L1 inhibitors.

### Discussion

Androgens, of which testosterone is the most abundant in males, are well described to suppress inflammation and impair immune function (Bebo et al., J Immunol. 162:35-40, 1999; Gubbels Bupp et al., Front Immunol 9:1269, 2018; Kissick et al., Proc Natl Acad Sci USA 111:9887-9892, 2014; and Lin et al., J Neuroimmunol 226:8-19, 2010), potentially contributing to a male bias in the incidence of cancers of nonreproductive organs (Ashley, Br J Cancer 23:313-328, 1969; Cartwright et al., Brit J Haematology 118:1071-1077, 2002; Cook et al., Br J Cancer 101:855-859, 2009; Edgren et al., Eur J Epidemiol 27:187-196, 2012; Fish, Nat Rev Immunol 8:737-744, 2008; and Klein and Flanagan, Nat Rev Immunol 16:626-638, 2016). Despite low levels of testosterone in the serum of prostate cancer patients undergoing ADT, the metastatic tumor microenvironment remains enriched for sex hormones (Montgomery et al., Cancer Res 68:4447-4454, 2008)—the source of which is unclear. Resistance to immunotherapy has been a clinical challenge in advanced prostate cancer patients and common mechanisms of therapy resistance fail to explain the inability to induce durable anti-tumor T cell responses in this disease.

Notably, a strong indicator of response to immunotherapy is IFNG expression within the tumor (Ayers et al., J Clin Invest 127:2930-2940, 2017; Prat et al., Cancer Res 77:3540-3550, 2017; and Riaz et al., Cell 171:934-949.e916, 2017) and androgens can suppress IFNG (Kissick et al., Proc Natl Acad Sci USA 111:9887-9892, 2014). In this study, a paradigm was leveraged challenging clinical trial that reported long-term progression free survival in metastatic castration resistant prostate cancer patients to identify a novel mechanism of immunotherapy resistance.

The data presented here indicate that there is a favorable CD8 T cell state associated with therapeutic response to PD-1/PD-L1 blockade that is the sum of T cell receptor engagement and inhibition of T cell intrinsic AR signaling. Importantly, AR inhibition poised CD8 T cells to respond to PD-1 blockade but was insufficient on its own. Together, treatment of tumor bearing mice with ADT, AR inhibition, and anti-PD-L1 was sufficient to drive 50% tumor regression. Furthermore, it was found that AR binds to accessible chromatin regions in the Ifng loci. Upon dislodging AR with a small molecule, critical anti-tumor cytokines increased in tumor-associated CD8 T cells. Moreover, AR blockade with a small molecule or Ar-KO CD8 T cells prevented CD8 T cells from losing functionality including IFNγ production in a classic LCMV T cell exhaustion mouse model. Together, these data reveal a previously unknown T cell intrinsic role for AR regulation of IFNγ activity which limits anti-tumor immunity and T cell re-invigoration. Finally, the direct binding of AR to critical inflammatory gene enhancer regions provides insight into a mechanism of sexual dimorphism of immunity.

### Experimental Model

Patient samples. Metastatic castration resistant prostate cancer patients enrolled on clinical trial NCT02312557 (Graff et al., Oncotarget 7(33):52810-52817, 2016; and Graff et al., J Immunother Cancer 8(2), 2020) underwent biopsy of a metastatic lesion at Oregon Health & Science University (Portland, OR). All patients had progressive disease on enzalutamide. Response to immune checkpoint inhibitor was defined by sustained reduction in PSA from baseline throughout treatment with PD-1 blockade of >25%. All human investigations were carried out after approval by a local Human Investigations Committee and in accord with an assurance filed with and approved by the Department of Health and Human Services. Data has been anonymized to protect the privacy of the participants. Investigators obtained informed consent from each participant. For single cell RNAseq analysis, fresh needle biopsies prior to pembrolizumab infusion were collected from patients enrolled between September 2017-January 2019 (n=19) which included three responders and five non-responders. Success rate with quality single-cell libraries was 63%. Bulk RNAseq libraries were made from adjacent flash frozen biopsies.

Human Sample dissociation. Fresh isolated tumor samples were collected immediately upon biopsy and processed the same day. Tissue was first minced into small pieces using scalpel and transferred into a 15 ml tube followed by digestion at room temperature in a shaker at 180 rpm for 30 minutes in 1 mg/ml collagenase type IV (Worthington Biochemicals), 100 μg/ml hyaluronidase (Sigma-Aldrich) and 20 mg/ml DNase (Roche) in PBS. Cells were then further disrupted with a 1-cc syringe plunger through a 70-μm nylon cell strainer (BD Biosciences) and filtered to obtain a single cell suspension. Dissociated cells were stained with PE anti-human CD45 (Invitrogen, Clone H130) for 30 minutes at 4° C. and subsequently washed 3 times with PBS+1% FBS, resuspended, and counted for yield and viability by trypan blue.

Immune cell enrichment for single cell RNA-sequencing. To enrich for leukocytes, FACS-sorting of live (Fixable viability dye eF780 negative), CD45 positive T cells was performed on a BD Bioscience InFlux cell sorter. Fluidic pressure was minimized with PSI no greater than 7 and cells were sorted using a large flow nozzle. Sorted cells were collected into cold PBS+1% FBS. This strategy was used for 7 of 8 samples. One sample used a PE anti-human CD45 magnetic enrichment and release strategy (Stemcell Technologies).

RNAseq 10× Genomics library preparation and sequencing. The enriched immune cells are immediately proceeded for single cell RNAseq library preparation. Single cell capturing and cDNA library generation were performed using the 10× Genomics Chromium single cell 3′ library construction kit v 2 (catalog #120267) according to the manufacture's instruction. Libraries were pooled prior to sequencing based on estimated number of cells in each library as determined by flow cytometry cell counts. Sequencing was performed following 10× Genomics instructions using NextSeq (Illumina) at the Massively Parallel Sequencing Shared Resource (MPSSR) at OHSU.

RNA and DNA isolation from fresh frozen OCT samples and sequencing. OCT samples were first cut with cryostat to remove excessive OCT as much as possible. The remaining tissue block was cut at 50 μm/section and immediately transfer to RiboZol (VWR, Cat #: N580). The tissues were incubated in RiboZol at room temperature with shaking every 3 minutes until the tissues were completely homogenized. The homogenate was centrifuged at 4° C. for 10 minutes at 12,000×g and the supernatant was transferred to a new tube and proceeded with RNA isolation following manufacturer's instructions. DNA was isolated from the lower two layers after removal of the supernatant following the manufacturer's instructions. RNAseq library preparation and sequencing were performed by the Massively Parallel Sequencing Shared Resource (MPSSR) at OHSU. Whole Exome Sequencing (WES) was performed by Novogene at a depth of 100×.

Animals, tumor models and antibodies. C57BL/6 (stock #000664), RIP-mOVA (stock #005431), OTI (stock #003831), C57Bl/6; CD90.1 (also known as Thy1.1) congenic mice (stock #000406) were purchased from the Jackson Laboratory. P14; Thy1.1 transgenic mice were obtained from Dr. Susan Kaech's laboratory. All animals were maintained under specific pathogen-free conditions in the Oregon Health &Science University (Portland, OR) animal facility. Sexually mature 12-week old males were used for the Murine PB-Cre+ PtenL/L p53L/L Smad4L/L (PPSM) castration resistant prostate tumor model and 3′-Methylcholanthrene (MCA) 205 sarcoma tumor model studies. PPSM (gift of Ronald DePinho) and MCA-205 cells (gift of Suyu Shu, Cleveland Clinic) were propagated in vitro using complete media, RPMI 1640 (Lonza) containing 0.292 ng/ml glutamine, 100 U/ml streptomycin/penicillin, 0.1 mM non-essential amino acids, 1 mM sodium pyruvate, and 10 mM HEPES (Sigma-Aldrich). All cell lines were tested and confirmed to be mycoplasma and endotoxin-free using the MycoAlert Detection kit (Lonza). All culture media reagents were purchased from Hyclone Laboratories unless noted otherwise. Control rat IgG (mIgG1) and anti-PD-L1 (mIgG1, clone 80) antibodies were obtained from Medlmmune (Astra Zeneca) (Schofield et al., MAbs 13:1857100, 2021). Animals were randomly assigned to treatment cohorts, and tumors were 25 to 50 mm² (by two-dimension caliper measurement) at the start of treatment. Any animal with a tumor >150 mm² was euthanized per the guidelines from the Institutional Animal Care and Use Committee. No outliers were excluded from the data presented. All animal experiments were approved by the Institutional Animal Care and Use Committee of OHSU.

Tumor challenge, treatments and orchiectomy surgeries, 1×10⁶ PPSM or 0.5×10⁶ MCA-205 tumor cells were injected on the hind flank of 12-week old C57BL/6 male mice (8 animals per group for survival experiments or 3 per group for phenotyping experiments). On day 7 (survival) or day 14 (phenotyping) post tumor inoculation, the tumor-bearing mice were orchiectomized as previously described (Valkenburg et al., J Vis Exp (111):53984, 2016), or treated with 0.5 μg of degarelix by S.C. (subcutaneous) injection once every 14 days. On the same day, animals were started on enzalutamide diet (50 mg/kg in Purina chow 5053, Research Diet Inc. 0.25 mg/mouse/day) or control diet, and treated with 4 doses of 200 μg rat IgG or anti-PD-L1 3 days apart (see FIG. 8D). For CD8 depletion studies, 200 μg anti-CD8a (Clone 53-6.7, BioXcell) was given on day 7 and 11. All antibody injections were given i.p. For orthotopic tumor implantation in the prostate, 1×10⁶ PPSM cells were injected as previously described (Pavese et al., J Vis Exp e50873, 2013) and orchiectomy was performed during the same surgery. For tumor antigen specific experiments, 0.5×10⁶ MCA-205-OVA tumor cells were injected on both flanks of RIP-mOVA animals (4 to 5 animals per group). All males were treated with 0.5 μg of degarelix by S.C. injection at time of tumor injection. 7 days later, 1×10⁵ OTI; Thy1.1 CD8 T cells isolated from splenocytes of OTI; Thy1.1 animals were adoptively transferred by i.v. injection into the tumor bearing animals, and animals started on enzalutamide or control diet.

Lymphocyte isolation. Tumor draining dLN (inguinal), spleens and tumors were harvested the day after the 3^{rd} treatment with anti-PD-L1, or 12 days post adoptive transfer of OTI T cells into MCA-OVA tumor bearing animals. Tumor infiltrating lymphocytes (TIL) were isolated by dissection of tumor tissue into small fragments in a 50-cc conical tube followed by digestion at room temperature in a bacterial shaker at 180 rpm for 30 min in 1 mg/ml collagenase type IV (Worthington Biochemicals), and 20 mg/ml DNAse (Roche) in PBS. Cells were then further disrupted with a 1-cc syringe plunger through a 70-μm nylon cell strainer (BD Biosciences) and filtered to obtain a single cell suspension. Tumor draining lymph nodes (dLN) and spleens were processed to obtain single-cell suspensions using frosted ends of microscope slides. Spleens were incubated with ammonium chloride potassium lysing buffer (Lonza) for 3 min at room temperature to lyse red blood cells. Cells were rinsed with PBS containing 1% FBS and 4 mM EDTA.

Flow cytometry. Cells were incubated for 20 min on ice with e506 fixable viability dye and the following antibodies: TCR3 (H57-597), CD4 (RM4-5), CD8 (53-6.7), CD44 (IM7), PD-1 (J43) and Thy1.1 (HIS51). Intracellular proteins Ki67 (SolA15), IFNγ (XMG1.2), TNFα (MP6-CT22), Nur77 (12.14) and Granzyme B (NGZB) were detected using the Fixation/Permeabilization Solution kit from BD Biosciences. All antibodies and viability dyes were purchased from eBioscience, Biolegend, or BD Biosciences. Data were collected with a Fortessa™ flow cytometer (BD Biosciences) and analyzed using FlowJo software (Tree Star). Unless noted otherwise in the figure legend, cells were gated through live/TCRβ+/CD8+ gates for analysis.

In vitro activation and intracellular cytokine staining. Bulk TIL, splenocytes and/or blood were plated in 96-well plates and stimulated for 4-5 h with PMA (80 nmol) and ionomycin (1.3 μmol), SIINFEKL peptide (1 nM; SEQ ID NO: 16) or gp33 peptide (10 nM) in presence of brefeldin A (BFA). Cells were then stained for surface markers, fixed and permeabilized, and stained for intracellular cytokines.

NanoString sample preparation and processing. PPSM tumor bearing mice were treated with one dose of degarelix and started on enzalutamide or control diet on day 7 post tumor inoculation. One week later, tumors were harvested and processed to single cell suspension. For tumor samples, EpCam positive tumor cells were removed using PE positive selection kit (EasySep™, STEMCELL TECHNOLOGIES, Vancouver, Canada) after staining the samples with EpCam-PE antibody. Enriched tumor infiltrated T cells were used to isolate RNA using RNeasy Kit (Qiagen). 50 ng RNA from each sample was used to measure RNA expression of genes in the nCounter mouse immunology panel using nCounter SPRINT profiler.

Chromatin immunoprecipitation. Male splenic T cells were isolated using magnetic separation (Mouse total T cell EasySep™, STEMCELL) and plated in a dish coated with 5 μg/ml anti-CD3 (145-2C11) and 1.5 μg/ml anti-CD28 (37.51) antibodies (ebiosience) to activate T cells, and treated with DMSO or 2.5 μM enzalutamide. After 72 hours, the cells were harvested, and AR ChIP was performed using the iDeal ChIP kit for transcription factor from Diagenode. Briefly, cells were cross-linked using 1% formaldehyde, followed by chromatin isolation. The isolated chromatin was then sheared to obtain a size of 300 to 700 base pairs using Diagenode Bioruptor Pico sonicator. For immunoprecipitation, 250 μl of the sheared chromatin solution was incubated with 5 μg of anti-AR antibody (Sigma-Aldrich 17-10489) or control normal rabbit IgG antibody (Sigma-Aldrich 12-370) bound to protein A/G coated magnetic beads overnight at 4° C. with rotation. The immunoprecipitated protein-DNA complex was washed rigorously, followed by reverse-crosslinking and DNA elution. 2.5 μL of sheared chromatin (1% of input) from control as well as enzalutamide treated samples were processed separately for preparing input sample DNA. The chromatin regions were measured by qPCR in the eluted DNA samples. The primers to amplify OCRs in Ifng (forward: GGTGTTGCAAAGACCTAG (SEQ ID NO: 1), reverse: GCAGTCCTTTTAATTACCCTG (SEQ ID NO: 2)) gene were used in qPCR. qPCR signals from each treatment group were normalized to the signal from the corresponding 1% input sample. The relative abundance of the chromatin regions bound to AR was calculated by normalizing to IgG control.

CRISPR/Cas9 AR gene deletion in naïve CD8 T cells. Nave WT or P14 CD8 T cells were purified from spleens using magnetic separation (Mouse CD8 T cell EasySep™, STEMCELL). AR was then deleted in purified naïve CD8 T cells according to the detailed protocol developed by Ian Parish's group (Nussing et al., J Immunol 204:2308-2315, 2020). sgRNA targeting the murine ARgene (sgRNA 1: AATACTGAATGACCGCCATC (SEQ ID NO: 3); sgRNA 2: AGGCTTCCGCAACTTGCATG (SEQ ID NO: 4); sgRNA 3: ATTGCCCATCTTGTCGTCTC (SEQ ID NO: 5); sgRNA 4: GGGTGGAAAGTAATAGTCGA (SEQ ID NO: 6)) and the mouse genome nontargeting Ctrl sgRNA (5′-GCACUACCAGAGCUAACUCA-3′ (SEQ ID NO: 7)) were obtained from Synthego. Cas9 recombinant protein was obtained from IDT. Following electroporation of the Cas9/sgRNA complex into WT or P14 purified naïve CD8 T cells, cells were either put in culture for 3 days in plates coated with 5 μg/ml of anti-CD3 and anti-CD28 antibodies, or adoptively transferred in recipient animals.

LCMV Clone 13 experiment. Immediately after Cas9 and mouse AR or NT (non-targeting) sgRNAs were electroporated in naïve purified splenic male P14; Thy1.1 CD8 T cells, 1×10⁴ AR-ko, NT P14 or WT CD8 T cells were adoptively transferred into WT recipient male mice by i.v. injection. At the same time, 2×10⁶ PFU of LCMV Clone 13 was injected i.v. At day 7 post adoptive transfer and LCMV inoculation, animals were bled, red blood cells lysed with ACK buffer, cells were stimulated for 6 hours with 10 μM gp33 peptide, and analyzed by flow cytometry. One animal per group was euthanized, adoptively transferred P14; Thy1.1 were sorted from the spleen based on Thy1.1 expression, RNA was extracted, and AR levels assessed by qPCR. At day 18, animals adoptively transferred with WT CD8 T cells were treated with 0.5 μg degarelix by S.C injection and put on enzalutamide diet for the rest of the experiment. On day 32, spleens and LN were harvested, stimulated with 10 μM gp33 peptide, and analyzed by flow cytometry.

Healthy human donor PBMCs and human AR RT-qPCR. Donor deidentified PBMC were from CMV/HIV/HBV seronegative male donors. PBMC were thawed, and untouched total T cells or CD8 T cells were purified via magnetic separation (human T cell or human CD8+ T cell, EasySep™, STEMCELL). T cells were stimulated for 0-3 days with plate bound anti-CD3 (OKT3, 4 μg/ml) and anti-CD28 (CD28.2, 2 μg/ml). Total RNA from unstimulated and TCR stimulated T cells was extracted (RNeasy, Qiagen) and subjected to one-step RT-qPCR for AR and SDHA (GoTaq 1-step RT-qPCR) amplified in a QuantStudio 3 thermocycler (Applied Biosystems). AR for each sample was internally normalized to SDHA, and data are reported as fold change versus AR expressed in unstimulated T cells.

Human qPCR primer sequences: human AR (forward: 5′CAGCAGAAATGATTGCACTATTGA3′ (SEQ ID NO: 8); reverse: 5′AGAGTCATCCCTGCTTCATAAC3′ (SEQ ID NO: 9)); human SDHA (forward: 5′CAGCACAGGGAGGAATCAAT3′ (SEQ ID NO: 10); reverse: 5′GTGTCGTAGAAATGCCACCT3′ (SEQ ID NO: 11)).

Mouse AR RT-qPCR. P14 T cells were sorted 7 days after adoptive transfer. Total RNA from unstimulated and TCR stimulated T cells, or sorted P14 was extracted (RNeasy, Qiagen) and subjected to one-step RT-qPCR for Ar and Gapdh (GoTaq 1-step RT-qPCR) amplified in a QuantStudio 3 thermocycler (Applied Biosystems). Mouse qPCR primer sequences: mouse Ar (forward: 5′GGAGAACTACTCCGGACCTTAT3′ (SEQ ID NO: 12); reverse: 5′GGGTGGAAAGTAATAGTCGATGG3′ (SEQ ID NO: 13)), mouse Gapdh (forward: 5′CTGGCCAAGGTCATCCAT3′ (SEQ ID NO: 14); reverse: 5′TTCTGGGTGGCAGTGATG3′ (SEQ ID NO: 15).

### Quantification and Statistical Analysis

Preprocessing of single cell RNAseq data. FASTQ files were mapped to human genome (hg19) and unique molecular identifier (UMI) counts quantified per gene per cell to generate a gene-barcode matrix using Cell Ranger software pipeline (version 2.1.1). To account for different sequencing depth of multiple libraries, reads of all samples were aggregated and libraries were normalized to the same sequencing depth using cellranger aggr function with normalize=mapped. The preprocessed matrix of gene counts versus cells contained 16,335 cells at an average sequencing depth of 7,655 reads per cell.

Unsupervised clustering of all cells. The preprocessed matrix generated by cellranger pipeline was imported into the Seurat (version 3.0.0) R (version 3.5.1) package (Satija et al., Nat Biotechnol 33:495-502, 2015). As a quality control (QC) step, cells with fewer than 100 genes were first filtered out and genes expressed in less than 0.1% of cells using zero as a cutoff for UMI counts were filtered out. Additional cells were removed based on mitochondrial gene content, UMI counts, and gene counts (mitochondrial % counts >=0.1, UMI counts >9000, gene counts >2500). The filtered gene expression matrix (14,609 genes and 16,044 cells) was normalized using NormalizeData function with ‘LogNormalize’ normalization method and ‘scale.factor’ equal to 10,000. Prior to dimension reduction and clustering analysis, the data was scaled and regressed out the effects of variation of UMI counts and percent mitochondrial contents. Furthermore, genes were focused on that exhibited high cell-to-cell variation and identified 1,608 genes using the FindVariableFeatures function in the Seurat package with ‘mean.var.plot’ method (mean cutoff between 0.0125 and 8, and dispersion over 0.5). Principal components analysis was performed on the scaled data cut to variable genes and the first 20 principal components were selected for downstream analysis, based on the elbow point on the plot of standard deviations of principal components. Cells were embedded in a shared nearest neighbor (SNN) graph constructed on the selected principal components and partitioned into clusters using FindClusters function with the resolution parameter set to 0.6 and the other parameters left as default. To visualize cells in two dimensions, uniform manifold approximation and projection (UMAP) was generated using RunUMAP function with the same principal components used in clustering analysis. Throughout the analysis, the absence of batch effects introduced by samples or other technical factors were confirmed, and thus batch effect removal was not performed on the data.

This analysis yielded 17 clusters. Cell types were identified based on the enrichment of a set of canonical markers for each cluster. A total of nine T cells clusters were annotated, one NK cell cluster, two myeloid cells clusters, one B cells cluster, one plasma cells cluster, one tumor cells cluster, and two fibroblast cells clusters. At the all-cells clustering stage, distinct cell types were not intended to be identified in detail and therefore clusters were merged into tumor cells and major lymphoid and myeloid immune cell subsets. The fraction of cells in each sample assigned to a given cluster c was computed, and Student's t-test was used to determine if there was a significant difference between responders and non-responders samples for cluster c. Percentage was calculated out of all cells without accounting for tumor cells.

Unsupervised clustering of T and NK cells. To reveal different cell types in T/NK lymphocytes, all cells classified as T and NK cells in the all-cells clustering analysis were extracted. The expression matrix of these cells was extracted from the preprocessed matrix (cellranger output), and analyzed through Seurat following the exact steps described above. This analysis used 1,496 variable genes, top 14 principal components, and a resolution of 0.5 in FindClusters. The fraction of cells in each sample assigned to a given cluster c was computed, and Student's t-test was used to determine if there was a significant difference between responders and non-responders samples for cluster c.

Unsupervised clustering of CD8 and CD4 T cells. To identify CD8 T cells states associated with response or resistance, all single cells classified as CD8 T cells in the T/NK-cells clustering analysis were extracted, the expression matrix from the preprocessed matrix (cellranger output) was subset, and these cells were processed using Seurat following the exact steps described above. Principal components analysis was performed on the scaled data cut to 1,728 variable genes and the top 14 principal components were used to generate UMAP for cell visualization. k-means clustering was performed on the top 50 principal components and CD8 T cells were classified into two clusters. A similar clustering analysis was performed on CD4 T cells, which used 2,042 variable genes.

Differential gene expression analysis and marker gene identification. For all single-cell differential gene expression tests, Wilcoxon rank-sum test implemented in Seurat was used. The differentially expressed genes for each cluster compared with all other cells were identified using FindAllMarkers function. Differential gene expression testing was also performed using FindMarkers function between responder cells (CD8_R) and non-responder cells (CD8_NR), and between favorable cells (CD8_F) and unfavorable cells (CD8_U). To identify top differentially expressed genes, an expression difference of at least 1.25 times fold change (ave_log FC>=log(1.25)) was required and an adjusted P value of <=0.05 (p_val_adj) with gene expression detected in at least 10% of cells in either one of the two comparison groups. The top 20 highly and differentially expressed genes, as ranked by the average fold change, were selected and scaled expression data of these genes was visualized in heatmaps.

Pathway enrichment analysis of single cells. To compute the gene expression signature which required fold change and P value, the output of Seurat FindMarkers or FindAllMarkers function was used with the following parameters: log fc.threshold=log(1), min.pct=0.001, and others set to default. This allowed the interrogation of the fold change and P value for all genes that were expressed in at least 0.1% of cells in either comparison group. The gene expression signature was calculated using the following formula: ave_log FC*log 10(1/(p_val+1e-300)), where ave_log FC and p_val were the outputs from Seurat. The gene expression signature of each comparison was imported into Camera to identify enriched pathways (Wu and Smyth, 2012. Nucleic Acids Res 40:e133, 2012) and the C2 canonical pathway reactome was used from the MSigDB database (version 7.0).

Master regulator analysis of single cells. Transcription factor activity was inferred using the master regulator (MR) inference algorithm (MARINa) (Lefebvre et al., Mol Syst Biol 6:377, 2010) compiled in the viper R package (Alvarez et al., Nat Genet 48:838-847, 2016). Gene expression signature and a regulatory network (regulome) are the two sources of data required as input for viper analysis. Gene expression signature was computed as described above. The transcription factor regulome used in this study was curated from several databases as previously described (Robertson et al., Cancer Cell32:204-220 e215, 2017).

NanoString nCounter data analysis. NanoString nCounter data was normalized and gene expression fold change was calculated using nSolver software from NanoString (version 4.0). A gene was nominated as differentially expressed when the fold change between a comparison group was greater than 1.2 and the gene raw count was above the background threshold for all samples. To determine whether genes upregulated in TIL-enza vs control were enriched in CD8_R single cells, GSEA Preranked tool was used to perform gene set enrichment analysis (GSEA, version 4.0.3) (Subramanian et al., Proc Natl Acad Sci USA 102:15545-15550, 2005), with the ranked gene list computed as described above and a permutation number of 3,000.

Whole transcriptome analysis of bulk tumor samples. FastQC v0.11.8 software (online at bioinformatics.bbsrc.ac.uk/projects/fastqc/) was used to determine the quality of raw fastq files. Sequencing reads were aligned to hg19 human reference genome and per-gene counts and TPM (Transcripts Per Kilobase Million) quantified by RSEM (1.3.1) (Li and Dewey, BMC Bioinformatics 12:323, 2011) based on the gene annotation gencode.v19.annotation.gtf. The regulon activity of androgen receptor (AR) for each bulk sample was inferred using single-sample VIPER analysis with TPM gene expression as input (Alvarez et al., Nat Genet 48:838-847, 2016). The CD8 R.vs.NR score of each bulk sample was calculated using z-score method. In brief, gene expression values (Log 2(TPM+1)) of each sample were converted to z-scores by: z=(x−μ)/σ, where μ is the average Log 2(TPM+1) across all samples of a gene and σ is the standard deviation of the Log 2(TPM+1) across all samples of a gene. The CD8 R.vs.NR score of each sample was the difference between average z-score of all up-regulated genes in CD8 R.vs.NR and average z-score of all down-regulated genes in CD8.R.vs.NR. The CD8 k1.vs.k2 score was calculated in the same way.

Whole transcriptome analysis of mouse CD8 T cell Ar-knockout and control samples. Sequencing FASTQ files were aligned to the mouse reference genome (GRCh38.p6) using STAR (Dobin et al., Bioinformatics 29:15-21, 2013) (version 2.7.3a) with default parameters. The STAR output of the number of reads per gene was used to quantify the expression level of each gene for downstream analysis. The Broad Institute GSEA software (Subramanian et al., Proc Natl Acad Sci USA 102:15545-15550, 2005) was used to determine the enrichment of the interferon gamma response pathway from the MSigDB hallmarks gene sets. The CD8 R.vs.NR score of each sample was calculated using z-score method as described above.

Tumor mutational burden. Whole exome sequencing (WES) reads were aligned against the GrCH37d5 genome using the Sanger cgpmap workflow (online at github.com/cancerit/dockstore-cgpmap) with realignment around indels and base recalibration performed using the Open Genomics GATK cocleaning workflow (online at github. com/OpenGenomics/gatk-cocleaning-tool). Somatic variants were called using a collection of callers via the mc3 workflow (github.com/opengenomics/mc3) (Ellrott et al., Cell Syst 6:271-281 e277, 2018), retaining all variants produced by Pindel and all variants reported by two or more tools that were not overlapped by a Pindel variant. The Mbp of genome covered by WES was determined using bedtools genomecov (V. 2.26.0), where any base pair covered by at least six aligned reads was considered covered. Coverage-adjusted tumor mutational burden was calculated on a per-sample basis by dividing the total number of somatic variants detected by the Mbp of genome covered.

Analysis of public prostate and melanoma datasets. The West Coast Dream Team (WCDT) human mCRPC mRNA data (n=99) (Quigley et al., Cell 174:758-769. e759, 2018) was obtained here: (davidquigley.com/prostate.html). The Hugo cohort (Hugo et al., Cell 168:542, 2017) (n=27) was downloaded from GSE78220 and the Van Allen cohort (Van Allen et al., Science 350:207-211, 2015) (n=42) was downloaded from the database of Genotypes and Phenotypes (dbGAP) under the accession number phs000452.v2.p1. The gene expressions were quantified by the transcripts per kilobase million (TPM). The regulon activity of androgen receptor (AR) in each sample was calculated using VIPER as described above. The IFNG pathway gene set was downloaded from the MSigDB database (version 7.0). Single-sample IFNG activity was calculated using z-scores as described above. The CD8 R.vs.NR score of each bulk sample was calculated using z-score method as described above.

Mouse data statistical analysis. Statistical analysis was performed using unpaired two-tailed Student t test (for comparison between two groups), one-way ANOVA for multiple comparisons or log-rank (Mantel-Cox) test for survival curves using GraphPad Prism 6 (GraphPad Software). Error bars represent SEM unless noted otherwise in the Brief Description of the Drawings. Statistical tests and P values are specified for each panel in the respective Brief Description of the Drawings, and P values<0.05 were considered significant. Biological replicates (individual animals) for each experiment are indicated in the Brief Description of the Drawings.

Data and software availability. R version 3.5.1 was used in this study. The sequence data generated in this study will be deposited in the Gene Expression Omnibus (GEO).

### Example 2: Correlation of AR Activity and IFNG Pathway Score with CD8 R Vs. NR Signature Score, in CAR-T Cells

Single cell data (discovery cohort) available from Deng et al. (“Characteristics of anti-CD19 CAR T cell infusion products associated with efficacy and toxicity in patients with large B cell lymphomas” Nature Medicine 26:1878-1887, 2020) was examined, and correlation analyses was carried out with the CD8 R vs. NR signature described herein.

The Deng et al. data is from T cells (from CAR T cell product); there are 24 samples in the discovery cohort, which was used in the current analysis. In Deng et al., 24 patients with diffuse large b cell lymphoma were treated with CD19 CAR-T cells. The infusion bag was rinsed and single cell RNA sequence performed on the infusion product.

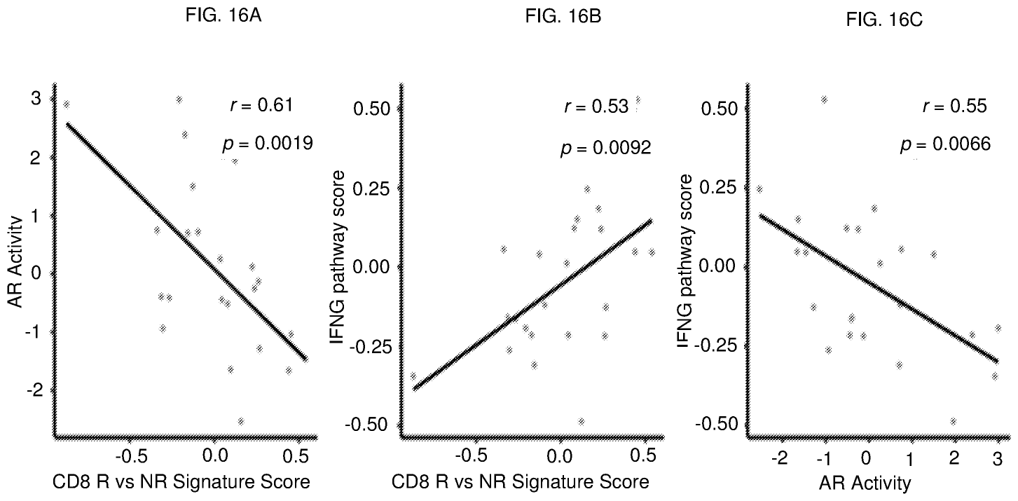

AR activity was compared to a CD8 R (responder) signature (FIG. 16A); INFG pathway activity by CD8 R signature (FIG. 16B); and IFNG activity inversely correlates with AR expression in CAR-T infusion product (FIG. 16C).

Strong and statistically significant correlations were found between AR activity and the CD8 R versus NR signature score; IFNG pathway score and CD8 R versus NR signature score; and between IFNG pathway score and AR activity.

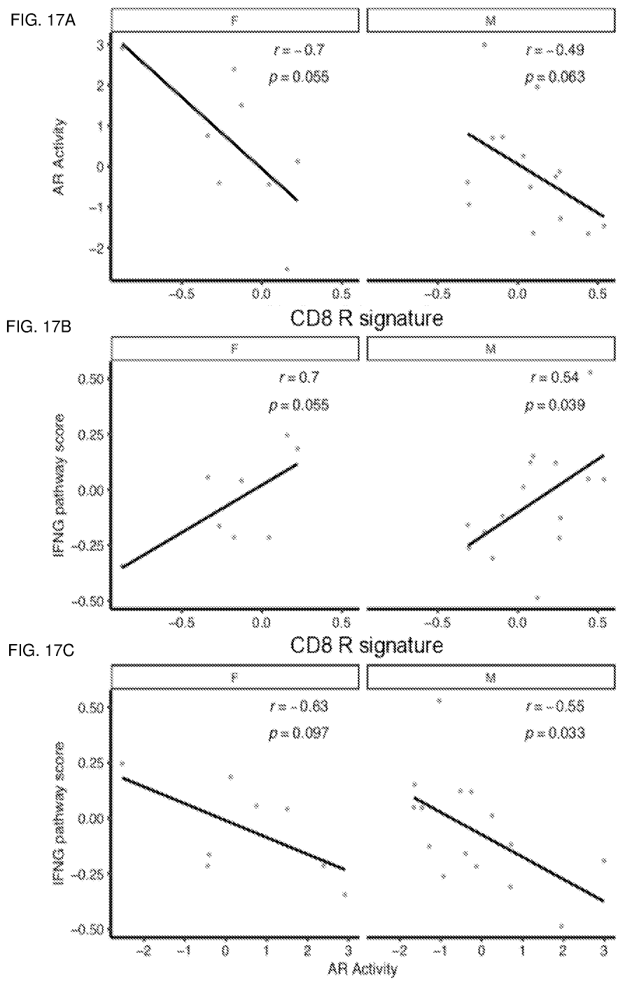

Given the interest in preforming AR-modified CAR-T therapy in prostate cancer patients, which are individuals of male sex, we re-evaluated the CD19 CAR-T data and segregated cell products based on sex (F=female; M=male). AR activity was compared to a CD8 R (responder) signature (FIG. 17A); IFNG pathway activity by CD8 R signature (FIG. 17B); and IFNG activity inversely correlates with AR expression in CAR-T infusion product (FIG. 17C).

### Example 3: Modulating Androgen Receptor Signaling to Enhance the Efficacy of CAR T Cell Therapy for Advanced Prostate Cancer

Immune checkpoint blockade has revolutionized the field of oncology. However, in advanced prostate cancer patients, immunotherapy treatments have largely failed. Notably, male sex in general is protective in autoimmunity (Whitacre, Nat Immunol. 2(9):777-780, 2001) and testosterone, the major androgen in males, is reported to be immunosuppressive. Inhibition of AR function may improve adoptive T cell therapies.

Adoptive cell transfer in the form of chimeric antigen receptor (CAR) T cell therapy has revolutionized the treatment of hematologic malignancies and is slowly making inroads into solid tumors. Challenges to the efficacy and safety of CAR T cell therapy in prostate cancer include antigen heterogeneity, an immunologically “cold” tumor microenvironment, poor persistence, and exhaustion. CAR T cell immunotherapy faces substantial challenges to efficacy in prostate cancer. Modulation of AR signaling in adoptively transferred CAR T cells and in prostate cancer cells by genetic and/or pharmacologic means may be a practical and facile approach to significantly improve antitumor responses.

Hypothesis: Blocking T cell intrinsic AR activity in CAR T cells will enhance antitumor efficacy in advanced prostate cancer.

Innovation: Significant innovation comes from the fundamental finding, described herein, that AR inhibition in T cells enhances responses to immune checkpoint inhibition in prostate cancer.

Evaluate the effect of AR modulation on STEAP1 CAR T cell effector function in vitro and in vivo. We will investigate how CAR T cell intrinsic AR loss modifies antitumor effects. We will perform either AR knockout (ko) by CRISPR/Cas9 genome editing or co-lentiviral transduction of CAR T cells with validated AR shRNA. These and control cell products will be tested in an established chronic antigen stimulation assay in vitro. CAR T cells will be phenotyped by flow cytometry and RNA-seq analysis to understand their differentiation states at baseline and with repeated antigen exposure.

Investigate tumor compartment-specific effects of AR modulation in CAR T cell tumor challenge experiments in syngeneic mouse prostate cancer models. This will provide insight into how T cell intrinsic or systemic AR modulation with CAR T cell therapy may impact the tumor microenvironment of prostate cancer. Using mouse prostate cancer models, mice will be treated after lymphodepleting cyclophosphamide with 1) untransduced T cells, 2) untransduced T cells with AR ko, 3) CAR T cells with AR ko, 4) untransduced T cells and enzalutamide, and 5) CAR T cells and enzalutamide. Readouts will include live bioluminescence imaging to follow tumor burden, peripheral CAR T cell persistence from retroorbital bleeds, serial serum cytokine analysis by Luminex assays, and tumor analysis by scRNA-seq, flow cytometry, and IHC to evaluate tumor immune composition.

Translational Goals and Anticipated Outcome(s): We predict that concomitant treatment with enzalutamide to modulate T cell intrinsic AR activity will enhance the efficacy of adoptively transferred CAR T cells. Based on the findings of these studies, future clinical studies may also incorporate CAR T cell products generated with AR ko by CRISPR/Cas9 genome editing.

### CLOSING PARAGRAPHS

As will be understood by one of ordinary skill in the art, each embodiment disclosed herein can comprise, consist essentially of or consist of its particular stated element, step, ingredient or component. Thus, the terms “include” or “including” should be interpreted to recite: “comprise, consist of, or consist essentially of.” The transition term “comprise” or “comprises” means has, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment. A material effect would cause a statistically significant change in the expected or intended improvement in immunotherapy effectiveness, for instances as measured by a statistically significant change in the level or activity of AR in a target or engineered cell.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context.

Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein.

Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents, printed publications, journal articles, other written text, and web site content throughout this specification (referenced materials herein). Each of the referenced materials are individually incorporated herein by reference in their entirety for their referenced teaching(s), as of the filing date of the first application in the priority chain in which the specific reference was included. For instance, with regard to chemical compounds and nucleic acid or amino acids sequences referenced herein that are available in a public database, the information in the database entry is incorporated herein by reference as of the date that the database identifier was first included in the text of an application in the priority chain.

It is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

Definitions and explanations used in the present disclosure are meant and intended to be controlling in any future construction unless clearly and unambiguously modified in the example(s) or when application of the meaning renders any construction meaningless or essentially meaningless. In cases where the construction of the term would render it meaningless or essentially meaningless, the definition should be taken from Webster's Dictionary, 11th Edition or a dictionary known to those of ordinary skill in the art, such as the Oxford Dictionary of Biochemistry and Molecular Biology, 2^{nd} Edition (Ed. Anthony Smith, Oxford University Press, Oxford, 2006), and/or A Dictionary of Chemistry, 8^{th} Edition (Ed. J. Law & R. Rennie, Oxford University Press, 2020).

What is claimed is:

1. A method of improving function of an immune cell or population of immune cells, comprising reducing intrinsic androgen receptor (AR) expression or activity level in the immune cell or in at least one cell in the population of immune cells.

2. The method of claim 1, wherein the immune cell or a cell within the population of immune cells is: a T-cell, a myeloid cell, a macrophage, a dendritic cell, a natural killer cell, or a B-cell.

3. The method of claim 1, wherein the cell is a T cell, and reducing the intrinsic AR level improves T cell function in allogenic T cell therapy, adoptive cell therapy, chimeric antigen receptor engineered T (CAR-T) cell therapy, or autologous T cell therapy.

4. The method of claim 1, wherein the cell is a T cell, and reducing the intrinsic AR level reduces regulatory T cell suppression.

5. The method of claim 1, wherein the cell is a myeloid cell or a macrophage, and reducing the intrinsic AR level improves engineered macrophage therapy.

6. The method of claim 1, wherein the cell is a dendritic cell, and reducing the intrinsic AR level improves antigen presentation and dendritic cell vaccine therapy.

7. The method of claim 1, wherein the cell is a natural killer cell, and reducing the intrinsic AR level improves cell killing.

8. The method of claim 1, wherein the cell is a B cell, and reducing the intrinsic AR level improves antibody maturation and vaccination.

9. An immune therapeutic cell genetically modified to reduce expression of intrinsic androgen receptor (AR).

10. The cell of claim 9, wherein the cell is a T-cell, a myeloid cell, a macrophage, a dendritic cell, a natural killer cell, or a B-cell.

11. A cell formulation comprising a cell of claim 9 or claim 10 and a pharmaceutically acceptable carrier.

12. A method of genetically-modifying an immune cell to provide an improved immune therapy product, the method comprising inhibiting expression and/or activity of androgen receptor (AR), in the immune cell.

13. The method of claim 12, wherein the method comprises inhibiting expression and/or activity of androgen receptor (AR) in at least one cell other than the immune cell.

14. A method of overcoming inhibition or failure of an immunotherapy response, comprising reducing expression and/or activity of androgen receptor (AR) in a cell used in the immunotherapy.

15. A method of improving the therapeutic effectiveness of an immunotherapy, comprising reducing expression and/or activity of androgen receptor (AR) in a cell used in the immunotherapy.

16. The method of any one of claims 12-15, wherein reducing expression and/or activity of androgen receptor (AR) in the cell comprises one or more of:
modification of a genomic sequence encoding AR, such as through targeted gene editing/disruption;
post-transcriptional inhibition of AR expression, such as through induction of RNAi; and/or
pharmacological inhibition or degradation of AR activity or function.

17. The method of claim 16, wherein the targeted gene editing comprises Clustered Regularly Interspaced Short Palindromic Repeats/CRISPR-associated (CRISPR/Cas) nuclease editing of a genomic sequence encoding AR, zinc finger nuclease (ZFN) editing of a genomic sequence encoding AR, or transcription activator like effector nuclease (TALEN) editing of a genomic sequence encoding AR.

18. A method of improving efficacy of immune checkpoint blockade therapy, comprising conducting the immune checkpoint blockade therapy (1) with a genetically-modifying cell in which the genetic modification results in reduction in androgen receptor (AR) expression and/or activity, or (2) in the presence of an AR agonist or antagonist.

19. The method of claim 18, wherein the immune checkpoint blockade therapy comprises cancer treatment.

20. The method of claim 19, wherein the immune checkpoint blockade therapy comprises treatment of prostate cancer or sarcoma.

21. Use of an immune cell engineered to have reduced androgen receptor (AR) expression and/or activity to treat cancer.

22. The use of claim 21, wherein the cancer comprises prostate cancer or sarcoma.

23. The use of claim 21 or claim 22, wherein the reduced AR expression and/or activity in the cell is accomplished through a method comprises one or more of:
modification of a genomic sequence encoding AR, such as through targeted gene editing/disruption;
post-transcriptional inhibition of AR expression, such as through induction of RNAi or ubiquitin mediated degradation; and/or
pharmacological inhibition of AR activity or function.

24. The use of claim 23, wherein the targeted gene editing comprises Clustered Regularly Interspaced Short Palindromic Repeats/CRISPR-associated (CRISPR/Cas) nuclease editing of a genomic sequence encoding AR, zinc finger nuclease (ZFN) editing of a genomic sequence encoding AR, or transcription activator like effector nuclease (TALEN) editing of a genomic sequence encoding AR.

25. A method, comprising:
dual inhibition of AR and an immune checkpoint, to reduce tumor growth and improve survival;
treatment with androgen deprivation therapy (ADT), AR inhibition (e.g., with enzalutamide, bicalutamide, flutamide, abiraterone, or the like) or AR degradation (e.g., with AR-PROTACs), and immune checkpoint blockade in a subject that has become refractory to checkpoint therapy; or
increasing the effectiveness of immune checkpoint blockade, by reducing AR activity in CD8 T cells in patients refractory to checkpoint therapy.

26. A method to increase production of effector cytokines (such as granzyme B, IFNγ and/or TNFα) in a subject, comprising treating the subject with:
an androgen reduction therapy (such as ADT, an AR inhibitor, an AR degrader, or a combination thereof); and
an anti-PD-L1 agent or other agent that provides immune checkpoint blockade.

27. The method of claim 26, wherein the androgen reduction therapy comprises treatment with a luteinizing hormone-releasing hormone (LHRH) inhibitor (such as degarelix) or orchiectomy.

28. A method to increase effectiveness of immune therapies and immune-mediated tumor comprising treating a subject with a combination of AR inhibitor with an epigenetic modifier (such as an EZH2 inhibitor or LSD1 inhibitor), alone or with immunotherapy.

29. A method, comprising treating a subject with an AR inhibitor, an epigenetic modifier, and immune cell therapy (such as autologous immune cell transfer).

30. A biomarker allowing identification of patients who could achieve a clinical benefit from checkpoint blockade, or to distinguish responsive from non-responsive patients, essentially as described herein.

31. The biomarker of claim 30, wherein the checkpoint blockade includes PD-L1 blockade.

32. The biomarker of claim 30, wherein the biomarker comprises: one or more of immune cell intrinsic AR activity, or CD8_R signature, or IFNG pathway activity.

33. The biomarker of claim 30, wherein the biomarker comprises expression level of at least one gene that is up-regulated in AR KO T cells, and/or expression level of at least one gene that is described herein as down-regulated in AR KO T cells.

34. The biomarker of claim 33, wherein the biomarker comprises expression level of at least two genes, at least three genes, at least four genes, at least five genes, at least six genes, at least seven genes, at least eight genes, at least nine genes, or more than nine genes that are up-regulated in AR KO T cells, and/or expression level of at least two genes, at least three genes, at least four genes, at least five genes, at least six genes, at least seven genes, at least eight genes, at least nine genes, or more than nine genes that are described herein as down-regulated in AR KO T cells.

35. The biomarker of claim 33 or claim 34, wherein:
the gene(s) up-regulated in AR KO T cells are selected from the group consisting of H2-Eb1, H2-Aa, 1113, Igfbp4, Rsad2, Rpl28-ps1, Gm1821, Tmem106a, Rpl36a-ps2, Ceacam-ps1, Gpr15, Trp53cor1, Rnaset2a, Gm10288, AL607105.1, and Emp1; or
the gene(s) down-regulated in AR KO T cells are selected from the group consisting of Traj24, Tm4sf19, Snord32a, AC159308.5, Gm15564, Gm36931, Gm45599, Gm6939, Gm26642, Arg1, Cxcl3, Gm590, Sema6b, 5830411N06Rik, Gm37768, Traj11, Gm38319, AC133083.2, Traj22, Gm45803, Gm17606, Klhl35, Gm26835, AC132474.1, Ankrd55, Ccl22, Gm37105 and 1110; or
both.

36. The biomarker of claim 30, comprising:
expression level of H2-Eb1, H2-Aa, 1113, Igfbp4, Rsad2, Rpl28-ps1, Gm1821, Tmem106a, Rpl36a-ps2, Ceacam-ps1, Gpr15, Trp53cor1, Rnaset2a, Gm10288, AL607105.1, and Emp1; or
expression level of Traj24, Tm4sf19, Snord32a, AC159308.5, Gm15564, Gm36931, Gm45599, Gm6939, Gm26642, Arg1, Cxcl3, Gm590, Sema6b, 5830411N06Rik, Gm37768, Traj11, Gm38319, AC133083.2, Traj22, Gm45803, Gm17606, Klhl35, Gm26835, AC132474.1, Ankrd55, Ccl22, Gm37105 and 1110; or
both.

37. Use of the biomarker of any one of claims 30-36 to develop or modify a treatment regimen for a subject.

38. A method of improving immune-cell mediated therapeutic treatment by reducing expression and/or function of androgen receptor, essentially as described herein.
